(19) United States
(12) Patent Application Publication
(10) Pub. No.: US 2009/0124997 A1
(43) Pub. Date: May 14, 2009

(54) METHODS AND DEVICES FOR IMPROVING DELIVERY OF A SUBSTANCE TO SKIN

(75) Inventors: Ronald J. Pettis, Cary, NC (US); Dians E. Sutter, Cary, NC (US); Richard P. Clarke, Raleigh, NC (US)

Correspondence Address: JONES DAY, 222 EAST 41ST ST, NEW YORK, NY, 10017, US

(73) Assignee: Becton, Dickinson and Company

(21) Appl. No.: 12/139,757

(22) Filed: Jun. 16, 2008

Related U.S. Application Data
(63) Continuation of application No. 11/072,844, filed on Mar. 3, 2005, now abandoned.
(60) Provisional application No. 60/550,896, filed on Mar. 3, 2004.

(51) Int. Cl. A61M 5/32

(52) U.S. Cl. 604/506

(57) ABSTRACT

A method of delivery of a substance to a human subject's skin comprising deposition into a specific compartment of the skin, wherein the delivery occurs at a controlled rate and pressure. The methods of the invention provide accurate deposition of s pre-selected volume of the substance, e.g., greater than 90% of the pre-selected volume. The methods of the invention encompass varying one or more parameters including but not limited to configurations of the delivery device, volume, pressure, and flow rate of delivery, to enhance the efficacy of delivery of the substance to the human skin. Substances delivered in accordance with the methods of the invention result in a more efficacious deposition of the substance into the targeted compartment, improved delivery performance, i.e., completeness of delivery as measured by quantification of the substance not delivered or the amount of the substance leaked out from the injection site, and enhanced safety as measured by the occurrence of minimal adverse cutaneous events at the site of injection.

This application is a continuation of application Ser. No. 11/072,844, filed Mar. 3, 2005, which claims the benefit of priority of U.S. Provisional Application Ser. No. 60/550,896, filed on Mar. 3, 2004, both of which are incorporated herein by reference in their entireties.

### 1. FIELD OF THE INVENTION

A method of delivery of a substance to a human subject's skin comprising deposition into a specific compartment of the skin, wherein the delivery occurs at a controlled rate and pressure. The methods of the invention provide accurate deposition of a pre-selected volume of the substance, e.g., greater than 90% of the pre-selected volume to a desired location. The methods of the invention encompass varying one or more parameters including but not limited to depth of deposition into the subject's skin, volume, pressure, and flow rate of delivery, to enhance the efficacy of delivery of the substance to the human skin. Substances delivered in accordance with the methods of the invention result in a more efficacious deposition of the substance into the targeted compartment, improved delivery performance, i.e., completeness of delivery as measured by quantification of the substance not delivered or the amount of the substance leaked out from the injection site, and enhanced safety as measured by the occurrence of minimal adverse cutaneous events at the site of injection.

### 2. BACKGROUND OF THE INVENTION

The importance of efficiently and safely administering pharmaceutical substances such as diagnostic agents and drugs has long been recognized. Although an important consideration for all pharmaceutical substances, obtaining adequate bioavailability of large molecules such as proteins that have arisen out of the biotechnology industry has recently highlighted this need to obtain efficient and reproducible absorption (Cleland et al., Curr. Opin. Biotechnol. 12: 212-219, 2001). The use of conventional needles has long provided one approach for delivering pharmaceutical substances to humans and animals by administration through the skin. Considerable effort has been made to achieve reproducible and efficacious delivery through the skin while improving the ease of injection and reducing patient apprehension and/or pain associated with conventional needles. Furthermore, certain delivery systems eliminate needles entirely, and rely upon chemical mediators or external driving forces such as iontophoretic currents or electroporation or thermal poration or sonophoresis to breach the stratum corneum, the outermost layer of the skin, and deliver substances through the surface of the skin. However, such delivery systems do, not reproducibly breach the skin barriers or deliver the pharmaceutical substance to a given depth below the surface of the skin and consequently, clinical results can be variable. Thus, mechanical breach of the stratum corneum, such as with needles, is believed to provide the most reproducible method of administration of substances through the surface of the skin, and to provide control and reliability in placement of administered substances.

Approaches for delivering substances beneath the surface of the skin have almost exclusively involved transdermal administration, i.e., delivery of substances through the skin to a site beneath the skin. Transdermal delivery includes subcutaneous, intramuscular or intravenous routes of administration of which, intramuscular (IM) and subcutaneous (SC) injections have been the most commonly used.

Anatomically, the outer surface of the body is made up of two major tissue layers, an outer epidermis and an underlying dermis, which together constitute the skin (for review, see Physiology, Biochemistry, and Molecular Biology of the Skin, Second Edition, L. A. Goldsmith, Ed., Oxford University Press, New York, 1991). The epidermis is subdivided into five layers or strata of a total thickness of between 75 and 150 μm. Beneath the epidermis lies the dermis, which contains two layers, an outermost portion referred to at the papillary dermis and a deeper layer referred to as the reticular dermis. The papillary dermis contains vast microcirculatory blood and lymphatic plexuses. In contrast, the reticular dermis is relatively acellular and avascular and made up of dense collagenous and elastic connective tissue. Beneath the epidermis and dermis is the subcutaneous tissue, also referred to as the hypodermis, which is composed of connective tissue and fatty tissue. Muscle tissue lies beneath the subcutaneous tissue.

As noted above, both the subcutaneous tissue and muscle tissue have been commonly used as sites for administration of pharmaceutical substances. The dermis, however, has rarely been targeted as a site for administration of substances, and this may be due, at least in part, to the difficulty of precise needle placement into the intradermal space. Furthermore, even though the dermis, in particular, the papillary dermis has been known to have a high degree of vascularity, it has not heretofore been appreciated that one could take advantage of this high degree of vascularity to obtain an improved absorption profile for administered substances compared to subcutaneous administration. This is because small drug molecules are typically rapidly absorbed after administration into the subcutaneous tissue which has been far more easily and predictably targeted than the dermis has been. On the other hand, large molecules such as proteins are typically not well absorbed through the capillary epithelium regardless of the degree of vascularity so that one would not have expected to achieve a significant absorption advantage over subcutaneous administration by the more difficult to achieve intradermal administration even for large molecules.

One approach to administration beneath the surface to the skin and into the region of the intradermal space has been routinely used in the Mantoux tuberculin test. In this procedure, a purified protein derivative is injected at a shallow angle to the skin surface using a 27 or 30 gauge needle (Flynn et al., Chest 106: 1463-5, 1994). A degree of uncertainty in placement of the injection can, however, result in some false negative test results. Moreover, the test has involved a localized injection to elicit a response at the site of injection and the Mantoux approach has not led to the use of intradermal injection for systemic administration of substances.

Some groups have reported on systemic administration by what has been characterized as “intradermal” injection. In one such report, a comparison study of subcutaneous and what was described as “intradermal” injection was performed (Autret et al, Therapie 46:5-8, 1991). The pharmaceutical substance tested was calcitonin, a protein of a molecular weight of about 3600. Although it was stated that the drug was injected intradermally, the injections used a 4 mm needle pushed up to the base at an angle of 60. This would have resulted in placement of the injectate at a. depth of about 3.5 mm and into the lower portion of the reticular dermis or into the subcutaneous tissue rather than into the vascularized papillary dermis. If, in fact, this group injected into the lower portion of the reticular dermis rather than into the subcutaneous tissue, it would be expected that the substance would either be slowly absorbed in the relatively less vascular reticular dermis or diffuse into the subcutaneous region to result in what would be functionally the same as subcutaneous administration and absorption. Such actual or functional subcutaneous administration would explain the reported lack of difference between subcutaneous and what was characterized as intradermal administration, in the times at which maximum plasma concentration was reached, the concentrations at each assay time and the areas under the curves.

Similarly, Bressolle et al. administered sodium ceftazidime in what was characterized as “intradermal” injection using a 4 mm needle (Bressolle et al., J. Pharm. Sci. 82:1175-1178, 1993). This would have resulted in injection to a depth of 4 mm below the skin surface to produce actual or functional subcutaneous injection, although good subcutaneous absorption would have been anticipated in this instance because sodium ceftazidime is hydrophilic and of relatively low molecular weight.

Another group reported on what was described as intradermal drug delivery device (U.S. Pat. No. 5,007,501). Injection was indicated to be at a slow rate and the injection site was intended to be in some region below the epidermis, i.e., the interface between the epidermis and the dermis or the interior of the dermis or subcutaneous tissue. This reference, however, provided no teachings that would suggest a selective administration into the dermis nor did the reference suggest any possible pharmacokinetic advantage that might result from such selective administration.

Thus there remains a continuing need for efficient and safe methods and devices for administration of pharmaceutical substances.

### 3. SUMMARY OF THE INVENTION

The present invention relates to a method of delivery of a substance to a human subject's skin comprising deposition into a specific compartment of the skin wherein delivery is performed at a controlled rate and pressure, so that greater than 90% of the injected volume is deposited in the pre-selected compartment of the skin. The methods of delivery of the invention provide accurate deposition of a pre-selected volume of the substance (e.g., greater than 90% volume of the pre-selected volume) to a pre-selected depth of the subject's skin. The invention is based, in part, on the inventors' discovery that varying one or more parameters including but not limited to the depth, volume, pressure, flow rate of delivery, significantly alters the efficacy of delivery of the substance to the human skin. Substances delivered in accordance with the methods of the invention result in a more efficacious deposition of the substance into the targeted compartment and improved delivery performance, e.g., completeness of delivery as measured by quantification of the substance not delivered or the amount of the substance leaked out from the injection site. A complete injection as used herein refers to an injection where greater than 90% of the pre-selected volume is delivered as determined by gravimetric methods known to one skilled in the art. Improved delivery performance encompasses an enhancement in one or more desired outcomes including but not limited to a biological, therapeutic and/or prophylatic effect of the substance delivered, an improvement in circulatory and/or tissue pharmacodynamics and/or pharmacokinetics.

The present invention provides an improved method of delivery of a substance to a subject's skin, in that it provides among other benefits, an efficient and consistent deposition of the substance at a pre-selected depth or compartment of the subject's skin, enhanced subject compliance due to minimal to no pain perception (as measured for example using a Gracely Box Scale and other methods known in the art and exemplified herein), improved pharmacokinetics and improved bioavailability, enhanced safety of delivery as measured for example by the occurrence of minimal adverse cutaneous events (e.g., Draize edema, erythema, bruising, discoloration, cuts) at the site of injection, improved tissue bioavailability, and improved tissue pharmacokinetics.

The invention encompasses a method of deposition of a substance to a human subject's skin, comprising deposition of the substance at a pre-selected depth within the subject's skin so that the substance is deposited within the pre-selected depth. The pre-selected depths that are targeted in accordance with the methods of the invention include but are not limited to a depth of at least 0.5 mm, at least 1.0 mm, at least 1.5 mm, at least 2.0 mm, or at least 3.0 mm.

Using the methods of the present invention, substances may be administered as a bolus, or by infusion. As used herein, the term “bolus” is intended to mean an amount that is delivered within a time period of less than or equal to ten (10) minutes. “Infusion” is intended to mean the delivery of a substance over a time period greater than ten (10) minutes. It is understood that bolus administration or delivery can be carried out with rate controlling means, for example a pump, or variable rate controlling means, for example user self-injection, manual injection.

The invention encompasses methods for improved bolus delivery of a substance to a subject's skin, preferably a human subject's skin, comprising delivering the substance over a period of no more than 10 minutes, and depositing the substance into a pre-selected compartment of the skin, wherein the delivery is performed at a controlled rate and at a pressure between 0.1 psi to 200 psi. In some embodiments, the substance is deposited at a depth of between 0.5 and 1.5 mm into the subject's skin and the pressure of delivery is between 5 psi and 200 psi. In other specific embodiments, the substance is deposited at a depth of between 2.0 and 3.0 mm into the subject's skin, and the pressure of delivery is between 0.1 psi and 50 psi. In yet other embodiments, the substance is deposited at a depth of between 1.5 mm and 2.0 mm into the subject's skin, and the pressure of delivery is between 5 psi and 150 psi.

The invention further encompasses methods for improved bolus delivery of a substance to a subject's skin, preferably a human subject's skin comprising: delivering the substance over a period of no more than 10 minutes; and depositing the substance into a pre-selected compartment of the skin, wherein the delivery is performed at a controlled pressure and at a rate up to 3500 μL/min. In some embodiments, the pressure of delivery is at least 10 psi and the flow rate is up to 1700 μL/min, so that the substance is deposited at a depth of between 0.5 mm to 2 mm into the skin. In other embodiments, the pressure of delivery is at least 15 psi and the flow rate is up to 2500 μL/min, so that the substance is deposited at a depth of between 0.5 mm to 2 mm into the skin. In yet other embodiments, the pressure of delivery is at least 20 psi and the flow rate is up to 3000 μL/min, so that the substance is deposited at a depth of between 0.5 mm to 2 mm into the skin. In other specific embodiments, the pressure of delivery is at least 10 psi and the flow rate is up to 1700 μL/min, so that the substance is deposited at a depth of between 2 mm to 3 mm into the skin. In other more specific embodiments, the pressure of delivery is at least 20 psi and the flow rate is up to 3500 μL/min, so that the substance is deposited at a depth of between 2 mm to 3 mm into the skin.

Device configurations that can be altered in accordance with the methods of the invention to achieve improved delivery of the substance include but are not limited to length of the needle, number of the needles, spacing between the needles, and relative exposed height of the needle outlet for targeting the specific compartment within the subject's skin. The invention encompasses altering such parameters so that the devices penetrates the targeted space within the subject's skin, allowing the skin to seal around the needle and preventing effusion of the substance onto the surface of the skin due to backpressure. The invention encompasses use of needle lengths capable of penetrations at depths of (i.e., exposed needle length) 1 mm, 1.25 mm, 1.5 mm, 2 mm, and 3 mm. In some embodiments, the invention encompasses microneedles ranging in length from 0.5 mm to 2 mm, from 0.5 mm to 3 mm, from 1 mm to 3 mm, or from 1 to 4 mm.

Devices that may be engineered in order to achieve optimal delivery in accordance with the methods of the invention include conventional injection needles, catheters or microneedles of all known types, employed singularly or in multiple needle arrays. The multiple needle arrays may comprise at least 2, at least 3, at least 6, up to at least 15 microneedles. In some embodiments, where a 34G steel cannula is used the array may comprise 1, 2, 3, 6 needles and up to 9 microneedles. In other embodiments, where the needle comprises silicon, the array may comprise at least 2 and up to 9 microneedles. In yet other embodiments, where the array comprises linear palladium arrays, the array may comprise at least 3 and up to 6 needles. The terms “needle” and “needles” as used herein are intended to encompass all such needle-like structures. The term “microneedles” as used herein are intended to encompass structures smaller than about 29 gauge, including 30 gauge but not including 29 gauge, typically about 31-50 gauge when such structures are cylindrical in nature. Non-cylindrical structures encompass by the term microneedles would therefore be of comparable diameter and include pyramidal, rectangular, octagonal, wedged, and other geometrical shapes. In some embodiments, the preferred needle size is a small Gauge hypodermic needle, commonly known as a 30 Gauge or 31 Gauge needle such as those disclosed in U.S. Pat. No. 6,569,143, which is incorporated herein by reference in its entirety.

The invention encompasses varying the volume of the substance delivered in order to improve deposition efficiency of the substance at the pre-selected depth of the subject's skin. In some embodiments, the volume of the substance delivered is kept constant, while one or more other parameters including but not limited to the depth of deposition in the subject's skin, infusion rate, pressure of delivery and application site are altered. The application site that may be used in the methods of the invention includes for example volar or upper arm, abdomen, deltoid or other aspect of the upper arm, thigh and back. In some embodiments, the volume of the substance delivered is varied as a function of pressure and pre-selected depth of delivery in the subject's skin. The invention encompasses varying the volume of the substance delivered so that at least 10 μL, at least 50 μL, at least 100 μL, at least 200 μL or at least 500 μL is deposited into the targeted compartment as measured for example using an absorbent swab method disclosed and exemplified herein. In some embodiments, the volume of the substance delivered is between 0.1 to 1 μL, 0.1 to 10 μL, 0.1 to 50 μL, or 0.1 to 100 μL.

In other embodiments, fluid flow rate is varied as a function of pressure and pre-selected depth of delivery in the subject's skin. In some embodiments, fluid flow rate is kept constant while one or more other parameters including but not limited to needle length, number of needles, spacing between needles, infusion rate, pressure of delivery and application site are altered. The invention encompasses varying the fluid rate from about 50 μL/min to 200 μL/min, 100 μL/min to 500 μL/min, 5 μL/hr to 5000 μL/min.

Rates of delivery may be controlled using pumping mechanism including but not limited to syringe pumps (e.g., Harvard Syringe Pumps), infusion pumps (e.g., microinfusion pumps), mechanical springs (e.g., coil springs, belleville springs, washers), elastomeric membrane, gas pressure devices, piezoelectric devices, electromotive based devices, or electromagnetic based devices, or any other device known in the art for controlling rates of delivery. Additionally any of the devices and methods disclosed in U.S. Pat. Nos. 5,957,895 and 6,074,369 may be used in accordance with the instant invention (the specified patents are incorporated herein by reference in their entireties).

Controlling rates of delivery, as used herein, refers to methods wherein the rate of delivery is the desired end point of the delivery process. The rate of delivery may be controlled using stringent as well as non-stringent means of control. Stringent means of control include without limitation methods whereby the rate of delivery is controlled by a mechanical system that operates within a specified range. Non-stringent means of control includes manual control wherein a skilled operator controls rate of delivery by perceptive feedback, e.g., syringe based systems, pens.

In yet another embodiment, pressure of delivery is varied as a function of needle pre-selected depth of delivery in the subject's skin. In some embodiments, pressure of delivery is kept constant while one or more other parameters including but not limited to needle length, number of needles, spacing between needles, infusion rate, volume of delivery and application site are altered. Pressure of delivery is measured using common methods known to one skilled in the art such as for example pressure transductions equipments as exemplified herein. Pressure of delivery of fluid may range from 10 psi to 15 psi, 10 psi to 20 psi, 10 psi to 30 psi. In yet other embodiments, pressure of delivery ranges from 10 to 50 psi, 20 psi to 200 psi, or 0.1 psi to 200 psi.

The methods of the invention encompass improving delivery of a substance to any compartment within the skin including but not limited to intradermal compartment, junctional layer, and the subcutaneous compartment. In some embodiments, the methods of the invention provide improving delivery of the substance to the intradermal compartment of a subject's skin. As used herein, intradermal is intended to mean administration of a substance into the dermis by placement of a substance predominately at a depth of at least about 0.3 mm, more preferably at least about 0.4 mm and most preferably at least about 0.5 mm up to a depth of no more than about 2.5 mm, more preferably, no more than about 2.0 mm and most preferably no more than about 1.7 mm which will result in rapid absorption of macromolecular and/or hydrophobic substances. Although not intending to be bound by a particular mechanism of action, the controlled delivery of a substance in this dermal space should enable an efficient outward migration of the substance to the undisturbed vascular and lymphatic microcapillary bed in the papillary dermis, where it can be absorbed into systemic circulation via these microcapillaries without being sequestered in transit by any other cutaneous tissue compartment.

In yet other embodiments, the methods of the invention encompass improving the delivery of the substance to the junctional layer of a subject's skin. As used herein, junctional layer refers to the transitory tissue space between the deepest layer of the dermis, i.e., the reticular dermis, and the hypodermis or the subcutaneous layer of the skin. In accordance with the methods of the invention, deposition of a substance into the junctional layer occurs predominately at a depth of at least about 1.5 mm, preferably, at least about 2 mm, up to a depth of no more than about 3 mm, preferably, no more than about 2.5 mm, which results in rapid absorption of the substance and reduced immune response.

In other embodiments, the methods of the invention encompass improving the delivery of the substance to the subcutaneous compartment of a subject's skin. Subcutaneous delivery encompasses deposition of the substance at a depth of at least 2.0 mm up to a depth of 3 mm or greater.

In certain applications, the methods of the invention may be employed to alter the pharmacokinetics (PK) and pharmacodynamics (PD) parameters of administered substances. The inventors, have found that by specifically targeting a selected compartment of the subject's skin and controlling the rate and pattern of delivery, the pharmacokinetics exhibited by specific drugs can be unexpectedly improved, and can in many situations be varied with resulting clinical advantage. Using the methods of the invention, by altering one or more parameters disclosed herein the pharmacokinetics of many substances including drugs and diagnostic substances, especially protein and peptide hormones, can also be altered, and in some cases improved. Potential corollary benefits include higher maximum concentrations for a given unit dose (Cₘₐₓ), higher bioavailability, more rapid uptake rates, more rapid onset of pharmacodynamics or biological effects, and reduced drug depot effects. According to the present invention, improved pharmacokinetics means increased bioavailability, decreased lag time (T_{lag}), decreased Tₘₐₓ, more rapid absorption rates, more rapid onset and/or increased Cₘₐₓ for a given amount of compound administered, compared to intramuscular or other non-IV parenteral means of drug delivery.

By bioavailability is meant the total amount of a given dosage that reached the blood compartment. This is generally measured as the area under the curve in a plot of concentration vs. time. By “lag time” is meant the delay between the administration of a compound and time to measure or detectable blood or plasma levels. Tₘₐₓ is a value representing the time to achieve maximal blood concentration of the compound, and Cₘₐₓ is the maximum blood concentration reached with a given dose and administration method. The time for onset is a function of T_{lag}, Tₘₐₓ and Cₘₐₓ, as all of these parameters influence the time necessary to achieve a blood (or target tissue) concentration necessary to realize a biological effect. Numerical values can be determined more precisely by analysis using kinetic models (as described below) and/or other means known to those of skill in the art.

The present invention improves the clinical utility of drugs, therapeutic agents, diagnostic agents, and other substances to humans or animals by accurately targeting the substance to a specific compartment of the skin. The methods employ devices engineered to accurately target a compartment of a subject's skin and to deliver substances to the skin as a bolus or by infusion. It has been discovered that the accurate placement of the device within the skin and delivering the substance at a controlled volume, rate and pressure provides for efficacious delivery and pharmacokinetic control of the substance. The devices are designed as to prevent leakage of the substance from the skin and improve adsorption within the targeted compartment. Another benefit of the invention is highly controllable dosing regimens and almost absolute control over the desired dosing regimen when delivery is coupled with a fluid control means or other control system to regulate metering of the drug or diagnostic agent into the body.

The methods of the invention provides an improved method of delivery of substances, in that it provides among other benefits, rapid uptake into the local lymphatics, improved targeting to a particular tissue, i.e., improved deposition of the delivered substance into the particular tissue, i.e., group or layer of cells that together perform a specific function, improved systemic bioavailability, improved tissue bioavailability, improved deposition of a pre-selected volume of the substance to be administered, improved tissue-specific kinetics (i.e., includes improved or altered biological pharmacodynamics and biological pharmacokinetics) rapid biological and pharmaco-dynamics (PD), and rapid biological and pharmacokinetics (PK).

### 4. DESCRIPTION OF THE DRAWINGS

FIG. 1 PEAK PRESSURE PER DEVICE. Pressure in the fluid path was measured via in line pressure transduction equipment. Peak pressure and sustaining (or average) pressure were recorded. The first two graphs show box plots of all peak pressure and average pressure measurements per treatment combination. There were 2 outlying/unusual observations for the peak pressure (represented as stars in the first graph below) and 2 outlying/unusual observations for the average pressure.

FIG. 2. DISTRIBUTION OF PAIN SCORES.

FIG. 3 CONFIDENCE INTERVALS FOR PAIN. Graphs show confidence intervals for pain at each time point per device and also the time by device interaction.

FIG. 4 NEEDLE DEVICE. An exploded perspective illustration of a needle assembly designed according to this invention.

FIG. 5 NEEDLE DEVICE. A partial cross-sectional illustration of the embodiment of FIG. 4.

FIG. 6 NEEDLE DEVICE. Embodiment of FIG. 4 attached to a syringe.

FIG. 7 ID INJECTION TECHNIQUE. A perspective view of one technique for making an ID injection.

FIG. 8 ID INJECTION TECHNIQUE. A perspective view of a 2^{nd} technique for making an ID injection.

FIG. 9 ID INJECTION TECHNIQUE. A perspective view of a 3rd technique for making an ID injection.

FIG. 10 ID INJECTION TECHNIQUE. A perspective view of a 4^{th} technique for making an ID injection.

FIG. 11 CONFIDENCE INTERVALS: Confidence intervals for pressure at average flow rate.

FIG. 12 DOT PLOTS FOR DISTRIBUTION OF PAIN SCORES

FIG. 13 CONFIDENCE INTERVALS: For needle stick pain and process pain per device.

FIG. 14 ACTUAL RECORDED LEAKAGE VOLUME. The following box plots show actual recorded leakage.

FIG. 15 PRESSURE MEASUREMENTS. The following box plots show the distribution of pressure measurement per treatment.

FIG. 16 MAIN EFFECTS PLOTS FOR TREATMENTS A-F: show the size and magnitude of the main effects.

FIG. 17 MAIN EFFECTS PLOTS FOR TREATMENTS E-H: show the size and magnitude of the main effects.

FIG. 18 BOX PLOTS FOR DISTRIBUTION OF PAIN AND UNPLEASANTNESS SCORES.

FIG. 19 INTERACTION PLOT. Graphs show the significant rate by time recorded interaction for pain scale intensity.

FIG. 20 CONFIDENCE INTERVALS. The following graphs show confidence intervals for average pain per device in original units.

FIG. 21 BOX PLOTS FOR DISTRIBUTION OF FLOW RATE MEASUREMENTS.

FIG. 22 MAIN EFFECTS PLOTS FOR TREATMENTS: show the size and magnitude of the significant device type and pressure effects.

FIG. 23. BOX PLOTS OF VOLUME LEAKED PER TREATMENT. Plots showing actual recorded leakage.

FIG. 24 BOX PLOTS OF PAIN SCORES PER TREATMENT.

FIG. 25 MAIN EFFECTS PLOTS FOR TREATMENTS: show the size and magnitude of the significant device type and pressure effects.

FIG. 26 CONFIDENCE INTERVALS FOR AVERAGE PAIN PER DEVICE.

FIG. 27 MAIN EFFECTS PLOT: Graphs of size and magnitude of the significant effects and interaction in ul/min.

FIG. 28 SIZE AND MAGNITUDE OF THE SIGNIFICANT MAIN EFFECTS.

FIG. 29 INTERACTION PLOT. Pressure×number of needle interactions

FIG. 30 PAIN AND FLOW RATE CORRELATION. The relationship between flow rate and pain scores.

FIG. 31 MAIN EFFECTS PLOTS. Graphs of size and magnitude of the significant effects and interaction

FIG. 32 INTERACTION PLOT.

FIG. 33 MAIN EFFECTS PLOTS. Graphs of size and magnitude of the significant effects and interaction

FIG. 34 MAIN EFFECTS PLOTS. Graphs of size and magnitude of the significant effects and interaction.

FIG. 35 INTERACTION PLOT. Site×needle length interaction

FIG. 36 PAIN AND FLOW RATE CORRELATION. The relationship between flow rate and pain scores.

FIG. 37 MAIN EFFECTS PLOTS. Graphs of size and magnitude of the significant effects and interaction

FIG. 38 BOX-COT PLOT FOR FLOW RATE.

FIG. 39. BOX PLOTS SHOWING DISTRIBUTION OF FLOW RATE MEASUREMENTS PER TREATMENT

FIG. 40. MAIN EFFECTS PLOTS. Graphs of size and magnitude of the significant effects and interaction.

FIG. 41 BOX PLOT OF ACTUAL LEAKAGE.

FIG. 42 BOX PLOT OF PAIN PER TREATMENT

FIG. 43 MAIN EFFECTS PLOTS. Graphs of size and magnitude of the significant effects and interaction.

FIG. 44 CONFIDENCE INTERVALS FOR AVERAGE PAIN PER DEVICE

FIG. 45 PAIN AND FLOW RATE CORRELATION

FIG. 46 SCHEMATICS OF INJECTION DEVICE

FIG. 47 SCHEMATICS OF INJECTION DEVICE

FIG. 48 SCHEMATICS OF INJECTION DEVICE

### 5. DETAILED DESCRIPTION OF THE INVENTION

The present invention relates to a method of delivery of a substance to a human subject's skin comprising deposition into a specific compartment of the skin wherein delivery is performed at a controlled rate and pressure, so that greater than 90% of the injected volume is deposited in the pre-selected compartment of the skin. The methods of delivery of the invention provide accurate deposition of a pre-selected volume of the substance (e.g., greater than 90% volume of the pre-selected volume) to a pre-selected depth of the subject's skin. The invention is based, in part, on the inventors' discovery that varying one or more parameters including but not limited to the depth, volume, pressure, and flow rate of delivery, significantly alters the efficacy of delivery of the substance to the human skin. Substances delivered in accordance with the methods of the invention result in a more efficacious deposition of the substance into the targeted compartment and improved delivery performance, i.e., completeness of delivery as measured by quantification of the substance not delivered or the amount of the substance leaked out from the injection site. A complete injection as used herein refers to an injection where greater than 90% of the pre-selected volume is delivered as determined by gravimetric methods known to one skilled in the art.

The present invention provides an improved method of delivery of a substance to a subject's skin, in that it provides among other benefits, an efficient and consistent deposition of the substance in to the targeted compartment, enhanced subject compliance due to minimal to no pain perception (as measured for example using a Gracely Box Scale and other methods known in the art and exemplified herein), improved pharmacokinetics and improved bioavailability, enhanced safety of delivery as measured for example by the occurrence of minimal adverse cutaneous events (e.g., Draize edema, erythema, bruising, discoloration, cuts) at the site of injection, improved tissue bioavailability, and improved tissue pharmacokinetics.

The invention encompasses varying the volume of the substance delivered in order to improve deposition efficiency of the substance. In some embodiments, the volume of the substance delivered is kept constant, while one or more other parameters including but not limited to needle length, number of needles, spacing between needles, infusion rate, pressure of delivery and application site are altered. The application site that may be used in the methods of the invention includes for example volar or upper arm, abdomen, deltoid or other aspect of the upper arm, thigh and back. In some embodiments, the volume of the substance delivered is varied as a function of pressure and needle length. The invention encompasses varying the volume of the substance delivered so that at least 10 μL, at least 50 μL, at least 100 μL, at least 200 μL or at least 500 μL is deposited into the targeted compartment as measured for example using an absorbent swab method disclosed and exemplified herein.

In other embodiments, fluid flow rate is varied as a function of pressure and microneedle length. In some embodiments, fluid flow rate is kept constant while one or more other parameters including but not limited to needle length, number of needles, spacing between needles, infusion rate, pressure of delivery and application site are altered. The invention encompasses varying the fluid rate from about 50 μL/min to 200 μL/min, 100 μL/min to 500 μL/min, 5 μL/hr to 5000 μL/min.

Rates of delivery may be controlled using pumping mechanism including but not limited to syringe pumps (e.g., Harvard Syringe Pumps), infusion pumps (e.g., microinfusion pumps), mechanical springs (e.g., coil springs, belleville springs, washers), elastomeric membrane, gas pressure devices, piezoelectric devices, electromotive based devices, or electromagnetic based devices, or any other device known in the art for controlling rates of delivery. Additionally any of the devices and methods disclosed in U.S. Pat. Nos. 5,957,895 and 6,074,369 may be used in accordance with the instant invention (the specified patents are incorporated herein by reference in their entireties)

In yet another embodiment, pressure of delivery is varied as a function of needle length. In some embodiments, pressure of delivery is kept constant while one or more other parameters including but not limited to needle length, number of needles, spacing between needles, infusion rate, volume of delivery and application site are altered. Pressure of delivery is measured using common methods known to one skilled in the art such as for example pressure transductions equipments as exemplified herein. Pressure of delivery of fluid may range from 10 psi to 15 psi, 10 psi to 20 psi, 10 psi to 30 psi. In yet other embodiments, pressure of delivery ranges from about 10 to about 50 psi, about 20 psi to 200 psi, or about 0.1 psi to 200 psi.

In order to achieve enhanced performance delivery of a substance in accordance with the methods of the invention, one or more factors including but not limited to the depth, volume, pressure, and flow rate of delivery of a substance may be varied and the response to each variation is evaluated by measuring completeness of the injected volume, the safety of the delivery as measured for example by adverse cutaneous events, including but not limited to Draize, edema, erythema, bruising, discoloration and cuts. The main objective would be to obtain the most efficacious delivery performance, i.e., completeness of injection as measured by quantification of the substance not delivered or the amount of substance leaked out form the injection site, while maintaining an enhanced subject compliance. As shown in Table 1 below, a grid-like analysis may be done in order to evaluate and assess the performance of the delivery. Once the response is evaluated one or more other factors may be further modified in order to achieve a better response rate.

TABLE 1
ANALYSIS OF RESPONSES

| | Needle | | | Needle |
|---|---|---|---|---|
| | Length | Site | Infusion Pressure | Number |
| Success of Injection | | | | |
| (inject at least 90% of | | | | |
| injected volume) | | | | |
| Flow rate | | | | |
| Pain | | | | |
| Wheal Formation | | | | |
| Fluid | | | | |
| Bleeding | | | | |

| | |
|---|---|
| Draize | Edema |
| | Erythema |

The invention encompasses methods for improved bolus delivery of a substance to a subject's skin, preferably a human subject's skin, comprising: delivering the substance over a period of no more than 10 minutes; and depositing the substance into a pre-selected compartment of the skin, wherein the delivery is performed at a controlled rate and at a pressure between 0.1 psi to 200 psi. In some embodiments, the substance is deposited at a depth of between 0.5 and 1.5 mm into the subject's skin and the pressure of delivery is between 5 psi and 200 psi. In other specific embodiments, the substance is deposited at a depth of between 2.0 and 3.0 mm into the subject's skin, and the pressure of delivery is between 0.1 psi and 50 psi. In yet other embodiments, the substance is deposited at a depth of between 1.5 mm and 2.0 mm into the subject's skin, and the pressure of delivery is between 5 psi and 150 psi.

The invention further encompasses methods for improved bolus delivery of a substance to a subject's skin, preferably a human subject's skin comprising: delivering the substance over a period of no more than 10 minutes; and depositing the substance into a pre-selected compartment of the skin, wherein the delivery is performed at a controlled pressure and at a rate up to 3500 μL/min. In some embodiments, the pressure of delivery is at least 10 psi and the flow rate is up to 1700 μL/min, so that the substance is deposited at a depth of between 0.5 mm to 2 mm into the skin. In other embodiments, the pressure of delivery is at least 15 psi and the flow rate is up to 2500 μL/min, so that the substance is deposited at a depth of between 0.5 mm to 2 mm into the skin. In yet other embodiments, the pressure of delivery is at least 20 psi and the flow rate is up to 3000 μL/min, so that the substance is deposited at a depth of between 0.5 mm to 2 mm into the skin. In other specific embodiments, the pressure of delivery is at least 10 psi and the flow rate is up to 1700 μL/min, so that the substance is deposited at a depth of between 2 mm to 3 mm into the skin. In other more specific embodiments, the pressure of delivery is at least 20 psi and the flow rate is up to 3500 μL/min, so that the substance is deposited at a depth of between 2 mm to 3 mm into the skin.

The methods of the invention encompass improving delivery of a substance to any compartment within the skin including but not limited to intradermal compartment, junctional layer, and the subcutaneous compartment. Mammalian skin contains two layers, as discussed above, specifically, the epidermis and dermis. The epidermis is made up of five layers, the stratum corneum, the stratum lucidum, the stratum granulosum, the stratum spinosum and the stratum geminativum and the dermis is made up of two layers, the upper papillary dermis and the deeper reticular dermis. The thickness of the dermis and epidermis varies from individual to individual, and within an individual, at different locations on the body. For example, it has been reported that the epidermis varies in thickness from about 40 to about 90 μm and the dermis varies in thickness ranging from just below the epidermis to a depth of from less than 1 mm in some regions of the body to just under 2 to about 4 mm in other regions of the body depending upon the particular study report (Hwang et al., Ann Plastic Surg 46:327-331, 2001; Southwood, Plasi. Reconsir. Surg 15:423-429, 1955; Rushmer et al., Science 154:343-348, 1966, each of which is incorporated herein by reference in their entireties).

In some embodiments, the methods of the invention provide improving delivery of the substance to the intradermal compartment of a subject's skin. As used herein, intradermal is intended to mean administration of a substance into the dermis in such a manner that the substance readily reaches the richly vascularized papillary dermis and is rapidly absorbed into the blood capillaries and/or lymphatic vessels to become systemically bioavailable. Such can result from placement of the substance in the upper region of the dermis, i.e., the papillary dermis or in the upper portion of the relatively less vascular reticular dermis such that the substance readily diffuses into the papillary dermis. Placement of a substance predominately at a depth of at least about 0.3 mm, more preferably, at least about 0.4 mm and most preferably at least about 0.5 mm up to a depth of no more than about 2.5 mm, more preferably, no more than about 2.0 mm and most preferably no more than about 1.7 mm will result in rapid absorption of macromolecular and/or hydrophobic substances. The controlled delivery of a substance in this dermal space below the papillary dermis in the reticular dermis, but sufficiently above the interface between the dermis and the subcutaneous tissue, should enable an efficient (outward) migration of the substance to the (undisturbed) vascular and lymphatic microcapillary bed (in the papillary dermis), where it can be absorbed into systemic circulation via these microcapillaries without being sequestered in transit by any other cutaneous tissue compartment.

In yet other embodiments, the methods of the invention encompass improving the delivery of the substance to the junctional layer of a subject's skin. As used herein, junctional layer refers to the transitory tissue space between the deepest layer of the dermis, i.e., the reticular dermis, and the hypodermis or the subcutaneous layer of the skin. As used herein, administration into the junctional layer is intended to encompass administration of a substance into the junctional layer in such a manner that the substance is deposited in the junctional layer such that it readily reaches the dense network of venous plexus and postcapillary veins of the junctional layer, and is rapidly absorbed and systemically distributed and/or transported to the lymphatic system. In accordance with the methods of the invention, deposition of a substance into the junctional layer occurs predominately at a depth of at least about 1.5 mm, preferably, at least about 2 mm, up to a depth of no more than about 3 mm, preferably, no more than about 2.5 mm, which results in rapid absorption of the substance and reduced immune response. In some embodiments, methods of the invention allow the penetration into the junctional layer of the subject's skin without passing through it. Delivering a substance into a subject's junctional layer in accordance with the methods of the invention results in improved pharmacokinetics, e.g., an improved pharmacokinetic profile.

In other embodiments, the methods of the invention encompass improving the delivery of the substance to the subcutaneous compartment of a subject's skin. Subcutaneous delivery encompasses deposition of the substance at a depth of at least 2.0 mm up to a depth of 3 mm or greater.

The methods of the invention provides an improved method of delivery of substances, in that it provides among other benefits, rapid uptake into the local lymphatics, improved targeting to a particular tissue, i.e., improved deposition of the delivered agent into the particular tissue, i.e., group or layer of cells that together perform a specific function, improved systemic bioavailability, improved tissue bioavailability, improved deposition of a pre-selected volume of the agent to be administered, improved tissue-specific kinetics (i.e., includes improved or altered biological pharmacodynamics and biological pharmacokinetics rapid biological and pharmaco-dynamics (PD), and rapid biological and pharmacokinetics (PK).

Substances delivered in accordance with the methods of the invention have improved tissue bioavailability in a particular tissue, including but not limited to, skin tissue, lymphatic tissue (e.g., lymph nodes), mucosal tissue, reproductive tissue, cervical tissue, vaginal tissue and any part of the body that consists of different types of tissue and that performs a particular function, i.e., an organ, including but not limited to lung, spleen, colon, thymus. In some embodiments, the tissue includes any tissue that interacts with or is accessible to the environment, e.g., skin, mucosal tissue. The invention encompasses any tissue or organ with a mucosal layer. Other tissues encompassed by the invention include without limitation Haemolymphoid System; Lymphoid Tissue (e.g., Epithelium-associated lymphoid Tissue and Mucosa-associated lymphoid Tissue or MALT (MALT can be further divided as organized mucosa-associated lymphoid Tissue (O-MALT) and diffused lymphoid tissue (D-MALT)); primary Lymphoid Tissue (e.g., thymus and bone marrow); Secondary Lymphoid Tissue (e.g., lymph node, spleen, alimentary, respiratory and Urigenital). It will be appreciated by one skilled in the art that MALT secondary includes gut associated lymphoid tissue (GALT); Bronchial associated lymphoid tissue (BALT), and genitourinary system. MALT specifically comprises lymph nodes, spleen, tissue associated with epithelial surfaces such as palentine and nasopharyngeal tonsils, Peyer's Patches in the small intestine and various nodules in the respiratory and urinogenital systems, the skin and conjunctivia of the eye. O-MALT includes the peripharyngeal lymphoid ring of the tonsils (palentine, lingual, nasopharyngeal and tubal), Oesophageal nodules and similar lymphoid tissue scattered throughout the alimentary tract from the duodenum to the anal canal. As used herein “tissue” refers to a group or layer of cells that together perform a function including but not limited to, skin tissue, lymphatic tissue (e.g., lymph nodes), mucosal tissue, reproductive tissue, cervical tissue, vaginal tissue and any part of the body that consists of different types of tissue and that performs a particular function, i.e., an organ, including but not limited to lung, spleen, colon, thymus. As used herein, tissue includes any tissue that interacts with or is accessible to the environment, e.g., skin, mucosal tissue.

As used herein, “tissue-bioavailability” means the amount of an agent (or substance) that is biologically available in vivo in a particular tissue. These amounts are commonly measured as activities that may relate to binding, labeling, detection, transport, stability, biological effect, or other measurable properties useful for diagnosis and/or therapy. In addition, it is understood that the definition of “tissue-bioavailability” also includes the amount of an agent available for use in a particular tissue. “Tissue-bioavailability” includes the total amount of the agent accumulated in a particular tissue, the amount of the agent presented to the particular tissue, the amount of the agent accumulated per mass/volume of particular tissue, and amount of the agent accumulated per unit time in a particular mass/volume of the particular tissue. Tissue bioavailability includes the amount of an agent that is available in vivo in a particular tissue or a collection of tissues such as those that make up the vasculature and/or various organs of the body (i.e., a part of the body that consists of different types of tissue and that performs a particular function. Examples include the spleen, thymus, lung, lymph nodes, heart and brain).

5.1 Delivery Devices

The present invention encompasses any device for accurately and selectively targeting a specific compartment of a subject's skin, including but not limited to the intradermal compartment, the junctional layer and the subcutaneous compartment. The nature of the device used is not critical as long as it penetrates the skin of the subject to the targeted depth within the without passing through it.

The invention compasses drug delivery devices and needle assemblies disclosed in U.S. Pat. No. 6,494,865 and U.S. patent application Ser. Nos. 10/357,502 and 10/337,413 (filed on Feb. 4, 2003 and Jan. 7, 2003, respectively), PCT application 2004/02783 filed Jan. 30, 2004; U.S. patent application Ser. No. 10/916,649 filed Aug. 12, 2004 all of which are incorporated herein by reference in their entireties.

In some embodiments, the device penetrates the skin at a depth within the intradermal space at a depth of at least about 0.5 mm, preferably at least 1.0 mm up to a depth of no more than 3.0 mm. Preferably the needle has a length sufficient to penetrate the intradermal space and an outlet at a depth within the intradermal space so that the substance is delivered and deposited therein. In general the needle is no longer than about 2 mm long, preferably 300 μm to 2 mm; most preferably 500 μm to 1 mm. The needle outlet is typically at a depth of about 250 μm to 2 mm when the needle is inserted in the skin, preferably at a depth of 750 μm to 1.5 and most preferably at a depth of about 1 mm.

In other embodiments device penetrates the skin at a depth within the junctional layer at a depth of at least about 2 mm, up to a depth of no more than about 3 mm, most preferably, no more than about 2.5 mm. In yet other embodiments, the device penetrates the skin at a depth within the subcutaneous compartment at a depth of at least 2.0 mm up to a depth of 3 mm or greater.

The invention encompasses use of devices designed for targeted delivery and encompasses microneedle-based injection and infusion systems or any other means to accurately target a specific compartment of a subject's skin. The invention also encompasses other delivery methods such, Mantoux-type ID injection, enhanced iontophoresis through microdevices, and direct deposition of fluid, solids, or other dosing forms into the skin.

Device configurations that can be altered in accordance with the methods of the invention to achieve improved delivery of the substance include but are not limited to length of the needle, number of the needles, spacing between the needles, and relative exposed height of the needle outlet for targeting the specific compartment within the subject's skin. The invention encompasses altering such parameters so that the devices penetrate the targeted space within the subject's skin, allowing the skin to seal around the needle and preventing effusion of the substance onto the surface of the skin due to backpressure. The invention encompasses use of needle lengths of 1 mm, 1.25 mm, 1.5 mm, 2 mm, and 3 mm. In some embodiments, the invention encompasses microneedles ranging in length from 0.25 mm to 2 mm, from 0.5 mm to 3 mm, from 1 mm to 3 mm, or from 1 mm to 4 mm.

Devices that may be engineered in order to achieve optimal delivery in accordance with the methods of the invention include conventional injection needles, catheters or microneedles of all known types, employed singularly or in multiple needle arrays. The multiple needle arrays may comprise at least 2, at least 3, at least 6, up to at least 15 microneedles. In some embodiments, where a 34G steel cannula is used the array may comprise 1, 2, 3, 6 needles and up to 9 microneedles. In other embodiments, where the needle comprises silicon, the array may comprise at least 2 and up to 9 microneedles. In yet other embodiments, where the array comprises linear palladium arrays, the array may comprise at least 3 and up to 6 needles. The terms “needle” and “needles” as used herein are intended to encompass all such needle-like structures. The term “microneedles” as used herein are intended to encompass structures smaller than about 29 gauge, typically about 30-50 gauge when such structures are cylindrical in nature. Non-cylindrical structures encompass by the term microneedles would therefore be of comparable diameter and include pyramidal, rectangular, octagonal, wedged, and other geometrical shapes. Microneedles used in the methods of the invention are also very sharp and of a very small gauge such as 30 or 34 G, to further reduce pain and other sensation during the injection or infusion. They may be used in the invention as individual single-lumen microneedles or multiple microneedles may be assembled or fabricated in linear arrays or two-dimensional arrays as to increase the rate of delivery or the amount of substance delivered in a given period of time. Microneedles may be incorporated into a variety of devices such as holders and housings that may also serve to limit the depth of penetration. The delivery devices of the invention may also incorporate reservoirs to contain the substance prior to delivery or pumps or other means for delivering the drug or other substance under pressure. Alternatively, the delivery devices may be linked externally to such additional components. In some embodiments, the preferred needle size is a small Gauge hypodermic needle, commonly known as a 30 Gauge or 31 Gauge needle such as those disclosed in U.S. Pat. No. 6,569,143, which is incorporated herein by reference in its entirety.

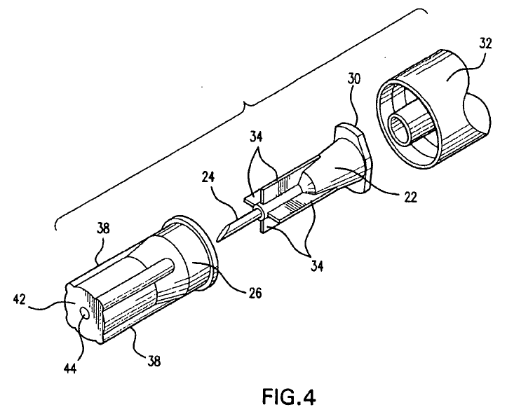

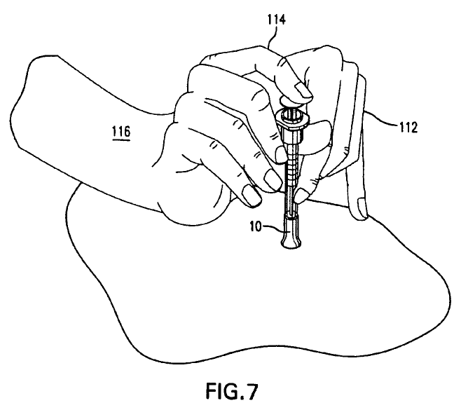

Exemplary devices are shown in FIGS. 4-6. FIGS. 4-6 of the drawings illustrate an example of a drug delivery device which can be used to practice the methods of the present invention for making intradermal injections illustrated in FIGS. 7-10. The device 10 illustrated in FIGS. 4-6 includes a needle assembly 20 which can be attached to a syringe barrel 60. Other forms of delivery devices may be used including pens of the types disclosed in U.S. Pat. No. 5,279,586, U.S. patent application Ser. No. 09/027,607 and PCT Application No. WO 00/09135, the disclosure of which are hereby incorporated by reference in their entirety. The method of the present invention can be used to intradermally inject substances, other than food, such as drugs, vaccines and the like used in the prevention, diagnosis, alleviation, treatment, or cure of disease into the skin of an animal such as a human, referred to collectively herein as “substances”.

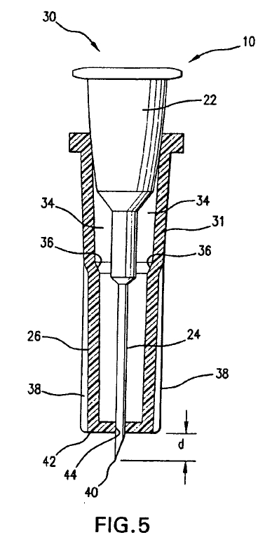

The needle assembly 20 includes a hub 22 that supports a needle cannula 24. The limiter 26 receives at least a portion of the hub 22 so that the limiter 26 generally surrounds the needle cannula 24 as best seen in FIG. 5.

One end 30 of the hub 22 is able to be secured to a receiver 32 of a syringe. A variety of syringe types for containing the substance to be intradermally delivered according to the present invention can be used with a needle assembly designed, with several examples being given below. The opposite end of the hub 22 preferably includes extensions 34 that are nestingly received against abutment surfaces 36 within the limiter 26. A plurality of ribs 38 preferably are provided on the limiter 26 to provide structural integrity and to facilitate handling the needle assembly 20.

By appropriately designing the size of the components, a distance “d” between a forward end or tip 40 of the needle 24 and a skin engaging surface 42 on the limiter 26 can be tightly controlled. The distance “d” preferably is in a range from approximately 0.5 mm to approximately 3.0 mm, and most preferably around 1.5 mm±0.2 mm to 0.3 mm. When the forward end 40 of the needle cannula 24 extends beyond the skin engaging surface 42 a distance within that range, an intradermal injection is ensured because the needle is unable to penetrate any further than the typical dermis layer of an animal. Typically, the outer skin layer, epidermis, has a thickness between 50-200 microns, and the dermis, the inner and thicker layer of the skin, has a thickness between 1.5-3.5 mm. Below the dermis layer is subcutaneous tissue (also sometimes referred to as the hypodermis layer) and muscle tissue, in that order.

As can be best seen in FIG. 5, the limiter 26 includes an opening 44 through which the forward end 40 of the needle cannula 24 protrudes. The dimensional relationship between the opening 44 and the forward end 40 can be controlled depending on the requirements of a particular situation. In the illustrated embodiment, the skin engaging surface 42 is generally planar or flat and continuous to provide a stable placement of the needle assembly 20 against an animal's skin. Although not specifically illustrated, it may be advantageous to have the generally planar skin engaging surface 42 include either raised portions in the form of ribs or recessed portions in the form of grooves in order to enhance stability or facilitate attachment of a needle shield to the needle tip 40. Additionally, the ribs 38 along the sides of the limiter 26 may be extended beyond the plane of the skin engaging surface 42.

Regardless of the shape or contour of the skin engaging surface 42, the preferred embodiment includes enough generally planar or flat surface area that contacts the skin to facilitate stabilizing the injector relative to the animal's skin. In the most preferred arrangement, the skin engaging surface 42 facilitates maintaining the injector in a generally perpendicular orientation relative to the skin surface and facilitates the application of pressure against the skin during injection. Thus, in the preferred embodiment, the limiter has dimension or outside diameter of at least 5 mm. The major dimension will depend upon the application and packaging limitations, but a convenient diameter is less than 15 mm or more preferably 11-12 mm.

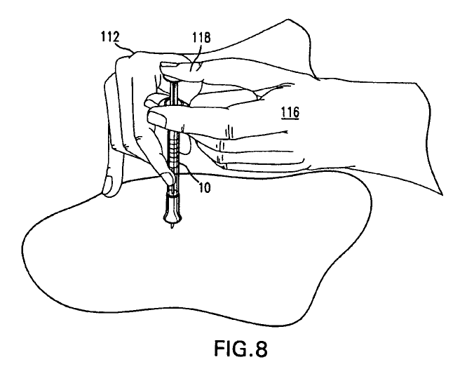

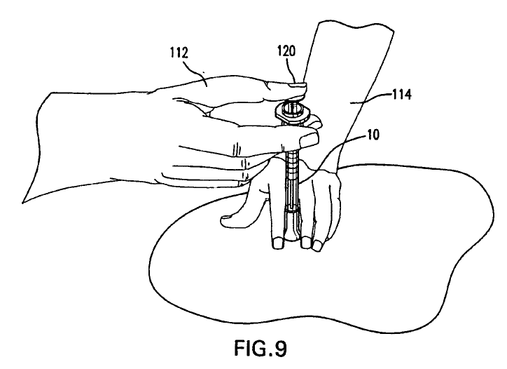

It is important to note that although FIGS. 4 and 5 illustrate a two-piece assembly where the hub 22 is made separate from the limiter 26, a device for use in connection with the invention is not limited to such an arrangement. Forming the hub 22 and limiter 26 integrally from a single piece of plastic material is an alternative to the example shown in FIGS. 8 and 9. Additionally, it is possible to adhesively or otherwise secure the hub 22 to the limiter 26 in the position illustrated in FIG. 5 so that the needle assembly 20 becomes a single piece unit upon assembly.

Having a hub 22 and limiter 26 provides the advantage of making an intradermal needle practical to manufacture. The preferred needle size is a small Gauge hypodermic needle, commonly known as a 30 Gauge or 31 Gauge needle. Having such a small diameter needle presents a challenge to make a needle short enough to prevent undue penetration beyond the dermis layer of an animal. The limiter 26 and the hub 22 facilitate utilizing a needle 24 that has an overall length that is much greater than the effective length of the needle, which penetrates the individual's tissue during an injection. With a needle assembly designed in accordance herewith, manufacturing is enhanced because larger length needles can be handled during the manufacturing and assembly processes while still obtaining the advantages of having a short needle for purposes of completing an intradermal injection.

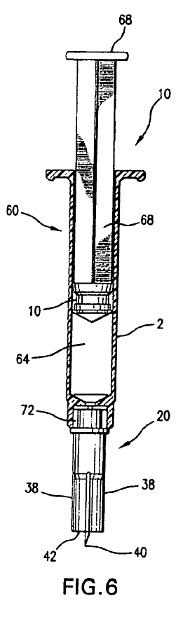

FIG. 6 illustrates the needle assembly 20 secured to a drug container such as a syringe 60 to form the device 10. A generally cylindrical syringe body 62 can be made of plastic or glass as is known in the art. The syringe body 62 provides a reservoir 64 for containing the substance to be administered during an injection. A plunger rod 66 has a manual activation flange 68 at one end with a stopper 70 at an opposite end as known in the art. Manual movement of the plunger rod 66 through the reservoir 64 forces the substance within the reservoir 64 to be expelled out of the end 40 of the needle as desired.

The hub 22 can be secured to the syringe body 62 in a variety of known manners. In one example, an interference fit is provided between the interior of the hub 22 and the exterior of the outlet port portion 72 of the syringe body 62. In another example, a conventional Luer fit arrangement is provided to secure the hub 22 on the end of the syringe 60. As can be appreciated from FIG. 6, such needle assembly designed is readily adaptable to a wide variety of conventional syringe styles.

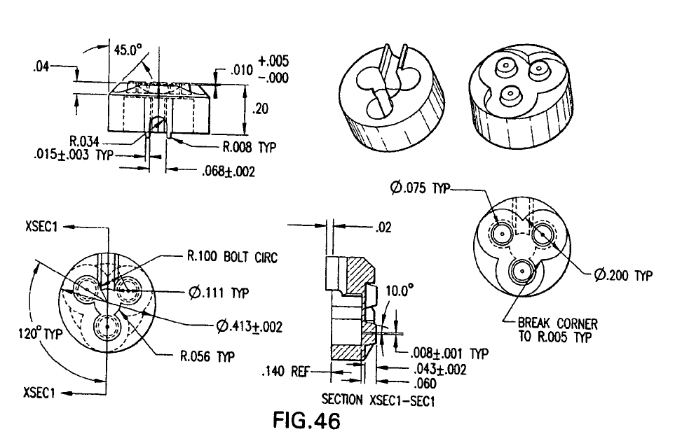

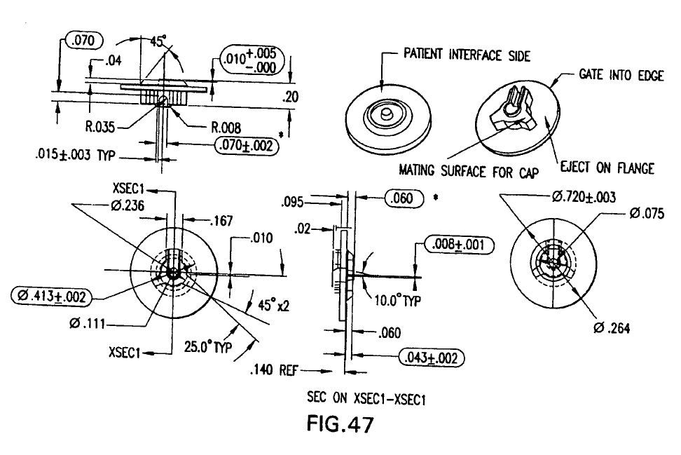

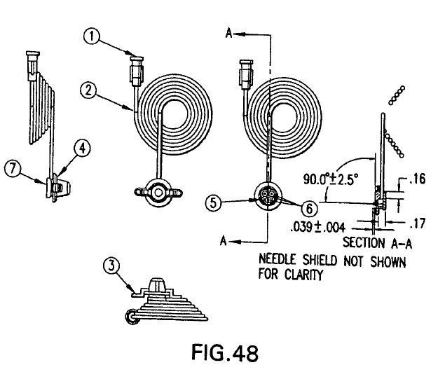

Alternative Devices that may be used in accordance with the invention are exemplified in FIGS. 46-48.

This invention provides an intradermal needle injector that is adaptable to be used with a variety of syringe types. Therefore, this invention provides the significant advantage of facilitating manufacture and assembly of intradermal needles on a mass production scale in an economical fashion.

The devices for use in the invention may comprise conventional injection needles, catheters or microneedles of all known types, employed singularly or in multiple needle arrays. The devices may comprise piezoelectric, electromotive, electromagnetic assisted delivery devices, gas-assisted delivery devices, of which directly penetrate the skin to provide access for delivery or directly deliver substances to the targeted location within the skin.

The length of microneedles are easily varied during the fabrication process and are routinely produced in less than 2 mm length. Microneedles are also a very sharp and of a very small gauge, to further reduce pain and other sensation during the injection or infusion. They may be used in the invention as individual single-lumen microneedles or multiple microneedles may be assembled or fabricated in linear arrays or two-dimensional arrays as to increase the rate of delivery or the amount of substance delivered in a given period of time. Microneedles may be incorporated into a variety of devices such as holders and housings that may also serve to limit the depth of penetration. The devices for use in the methods of the invention may also incorporate reservoirs to contain the substance prior to delivery or pumps or other means for delivering the drug or other substance under pressure.

The devices for use in accordance with the methods of the invention may comprise any means for controlling rates and/or pressures of delivery using pumping mechanism including but not limited to syringe pumps (e.g., Harvard Syringe Pumps), infusion pumps (e.g., microinfusion pumps), mechanical springs (e.g., coil springs, belleville springs, washers), elastomeric membrane, gas pressure devices, piezoelectric devices, electromotive based devices, or electromagnetic based devices, or any other device known in the art for controlling rates of delivery. Additionally any of the devices and methods disclosed in U.S. Pat. Nos. 5,957,895 and 6,074,369 may be used in accordance with the instant invention (the specified patents are incorporated herein by reference in their entireties).

5.2 Administration Methods

The present invention encompasses methods for delivery of substances described and exemplified herein to a specific compartment of a subject's skin, preferably a human subject, by accurate deposition of the substance into the targeted compartment, using controlled delivery parameters such as volume, infusion rate, and pressure of delivery. Preferably the methods of the invention result in accurate deposition of the substance into the targeted compartment without passing through it. Substances delivered in accordance with the methods of the invention result in a more efficacious deposition of the substance into the targeted compartment and improved delivery performance, e.g., completeness of delivery as measured by quantification of the substance not delivered or the amount of the substance leaked out from the injection site. The present invention provides an improved method of delivery of a substance to a subject's skin, in that it provides among other benefits, an efficient and consistent deposition of the substance in to the targeted compartment, enhanced subject compliance due to minimal to no pain perception (as measured for example using a Gracely Box Scale and other methods known in the art and exemplified herein), improved pharmacokinetics and improved bioavailability, enhanced safety of delivery as measured for example by the occurrence of minimal adverse cutaneous events (e.g., Draize edema, erythema, bruising, discoloration, cuts) at the site of injection, enhanced tissue bioavailability and enhanced tissue pharmacokinetics.

Once a formulation containing the substance to be delivered is prepared, the formulation is typically transferred to an injection device for skin delivery, e.g., a syringe. Delivery of the formulations of the invention in accordance with the methods of the invention also provides an improved therapeutic and clinical efficacy of the substance over conventional modes of delivery by enhancing the performance of delivery and deposition of the substance to the targeted compartment. The delivery methods of the invention provide benefits and improvements over conventional modes of delivery including but not limited to improved pharmacokinetics and bioavailability. In some embodiments, the methods of the invention allow administration of therapeutic substances to which the induction of an immune response would not be beneficial to the therapeutic effect of the substance to be delivered.

The formulations of the invention are administered using any of the devices and methods disclosed in U.S. patent application Ser. Nos. 09/417,671, filed on Oct. 14, 1999; 09/606,909, filed on Jun. 29, 2000; 09/893,746, filed on Jun. 29, 2001; 10/028,989, filed on Dec. 28, 2001; 10/028,988, filed on Dec. 28, 2001; or International Publication No.'s EP 10922 444, published Apr. 18, 2001; WO 01/02178, published Jan. 10, 2002; and WO 02/02179, published Jan. 10, 2002; all of which are incorporated herein by reference in their entirety. Non-limiting examples of devices that may be used in accordance with the methods of the invention are syringes, pen, pumps, catheters, and autoinjectors.

The methods of administration comprise microneedle-based injection and infusion systems or any other means to accurately target a compartment within the skin. The administration methods of the invention encompass not only microdevice-based injection means, but other delivery methods such as Mantoux-type intradermal injection, enhanced iontophoresis through microdevices, and direct deposition of fluid, solids, or other dosing forms into the skin. In a specific embodiment, the formulations of the invention are administered to an intradermal compartment of a subject's skin using an intradermal Mantoux type injection, see, e.g., Flynn et al., 1994, Chest 106: 1463-5, which is incorporated herein by reference in its entirety. In a specific embodiment, the formulation of the invention is delivered to the intradermal compartment of a subject's skin using the following exemplary method. The formulation is drawn up into a syringe, e.g., a 1 mL latex free syringe with a 20 gauge needle; after the syringe is loaded it is replaced with a 30 gauge needle for intradermal administration. The skin of the subject, is approached at the most shallow possible angle with the bevel of the needle pointing upwards, and the skin pulled tight. The injection volume is then pushed in slowly over 5-10 seconds forming the typical “bleb” and the needle is subsequently slowly removed. Preferably, only one injection site is used. More preferably, the injection volume is no more than 100 μL, due in part, to the fact that a larger injection volume may increase the spill over into the surrounding tissue space, e.g., the subcutaneous space.

The invention comprises microneedle based devices that may further comprise ballistic fluid injection devices, piezoelectric, electromotive, electromagnetic assisted delivery devices, gas-assisted delivery devices, which directly penetrate the skin to directly deliver the formulations of the invention to the targeted location within the skin.

The formulations delivered or administered in accordance with the invention include solutions thereof in pharmaceutically acceptable diluents or solvents, suspensions, gels, particulates such as micro- and nanoparticles either suspended or dispersed, as well as in-situ forming vehicles of same.

It has been found that certain features of the administration methods provide clinically useful PK/PD and dose accuracy. For example, it has been found that placement of the needle outlet within the skin significantly affects PK/PD parameters. The outlet of a conventional or standard gauge needle with a bevel has a relatively large exposed height (the axial length of the outlet). Although the needle tip may be placed at the desired depth within the intradermal space, the large exposed height of the needle outlet causes the delivered substance to be deposited at a much shallower depth nearer to the skin surface. As a result, the substance tends to effuse out of the skin due to backpressure exerted by the skin itself and to pressure built up from accumulating fluid from the injection or infusion. That is, at a greater depth a needle outlet with a greater exposed height will still seal efficiently where as an outlet with the same exposed height will not seal efficiently when placed in a shallower depth within the intradermal space. Typically, the exposed height of the needle outlet will be from 0 to about 1 mm. A needle outlet with an exposed height of 0 mm has no bevel and is at the tip of the needle. In this case, the depth of the outlet is the same as the depth of penetration of the needle. A needle outlet that is either formed by a bevel or by an opening through the side of the needle has a measurable exposed height it is understood that a single needle may have more than one opening or outlets suitable for delivery of substances to the dermal space.

It has also been found that by controlling the pressure of injection or infusion the high backpressure exerted during administration to the skin, can be avoided. By placing a constant pressure directly on the liquid interface a more constant delivery rate can be achieved, which may optimize absorption and obtain the improved pharmacokinetics. Delivery rate and volume can also be controlled to prevent the formation of wheals at the site of delivery and to prevent backpressure from pushing the dermal-access means out-of the-skin. The appropriate delivery rates and volumes to obtain these effects for a selected substance may be determined experimentally using methods disclosed and exemplified herein. Increased spacing between multiple needles allows broader fluid distribution and increased rates of delivery or larger fluid volumes.

The administration methods useful for carrying out the invention include both bolus and infusion delivery of drugs and other substances to humans or animals subjects. A bolus dose is a single dose delivered in a single volume unit over a relatively brief period of time, typically less than or equal to about 10 minutes. Infusion administration comprises administering a fluid at a selected rate that may be constant or variable, over a relatively more extended time period, typically greater than about 10 minutes.

Delivery from the reservoir into the skin may occur actively, with the application of pressure or other driving means. Examples of preferred pressure generating means include pumps, syringes, elastomeric membranes, gas pressure, piezoelectric, electromotive, electromagnetic pumping, or mechanical springs (e.g., Belleville springs or washers) or combinations thereof. If desired, the rate of delivery of the substance may be variably controlled by the pressure-generating means. As a result, the substance enters the skin and is absorbed in an amount and at a rate sufficient to produce a clinically efficacious result. As used herein, the term “clinically efficacious result” is meant a clinically useful biological response including both diagnostically and therapeutically useful responses resulting from administration of a substance or substances. For example, diagnostic testing or prevention or treatment of a disease or condition is a clinically efficacious result. Such clinically efficacious results include diagnostic results such as the measurement of glomerular filtration pressure following injection of insulin, the diagnosis of adrenocortical function in children following injection of ACTH, the causing of the gallbladder to contract and evacuate bile upon injection of cholecystokinin and the like as well as therapeutic results, such as clinically adequate control of blood sugar levels upon injection of insulin, clinically adequate management of hormone deficiency following hormone injection such as parathyroid hormone or growth hormone, clinically adequate treatment of toxicity upon injection of an antitoxin and the like.

The present invention provides a method for therapeutic and/or phrophylactic treatment by delivery of a drug or other substance to a human or animal subject by directly targeting a compartment of the subject's skin, where the drug or substance is deposited. Substances infused according to the methods of the invention have been found to exhibit pharmacokinetics superior to, and more clinically desirable than that conventional methods of delivery.

Exemplary modes of intradermal injections using exemplary devices are shown in FIGS. 7-10. Having described the intradermal delivery device including the needle assembly 20 and drug container 60 supra, its operation and use in practicing the methods of the present invention for intradermally injecting substances is described below.

Prior to inserting the needle cannula 24, an injection site upon the skin of the animal is selected and cleaned. Subsequent to selecting and cleaning the site, the forward end 40 of the needle cannula 24 is inserted into the skin of the animal at an angle of generally 90 degrees until the skin engaging surface 42 contacts the skin. The skin engaging surface 42 prevents the needle cannula 42 from passing through the dermis layer of the skin and injecting the substance into the subcutaneous layer.

While the needle cannula 42 is inserted into the skin, the substance is intradermally injected. The substance may be prefilled into the syringe 60, either substantially before and stored therein just prior to making the injection. Several variations of the method of performing the injection may be utilized depending upon individual preferences and syringe type. In any event, the penetration of the needle cannula 42 is most preferably no more than about 1.5 mm because the skin engaging surface 42 prevents any further penetration.

Also, during the administration of an intradermal injection, the forward end 40 of the needle cannula 42 is embedded in the dermis layer of the skin which results in a reasonable amount of back pressure during the injection of the substance. This back pressure could be on the order of 76 psi. In order to reach this pressure with a minimal amount of force having to be applied by the user to the plunger rod 66 of the syringe, a syringe barrel 60 with a small inside diameter is preferred such as 0.183″ (4.65 mm) or less. The method of this invention thus includes selecting a syringe for injection having an inside diameter of sufficient width to generate a force sufficient to overcome the back pressure of the dermis layer when the substance is expelled from the syringe to make the injection.

In addition, since intradermal injections are typically carried out with small volumes of the substance to be injected, e.g., on the order of no more than 0.5 mL, and preferably around 0.1 mL, a syringe barrel 60 with a small inside diameter is preferred to minimize dead space which could result in wasted substance captured between the stopper 70 and the shoulder of the syringe after the injection is completed. Also, because of the small volumes of substance, on the order of 0.1 ml, a syringe barrel with a small inside diameter is preferred to minimize air head space between the level of the substance and the stopper 70 during process of inserting the stopper. Further, the small inside diameter enhances the ability to inspect and visualize the volume of the substance within the barrel of the syringe.

As shown in FIG. 7, the syringe 60 may be grasped with a first hand 112 and the plunger 66 depressed with the forefinger 114 of a second hand 116. Alternatively, as shown in FIG. 8 the plunger 66 may be depressed by the thumb 118 of the second hand 116 while the syringe 60 is held by the first hand. In each of these variations, the skin of the animal is depressed, and stretched by the skin engaging surface 42 on the limiter 26. The skin is contacted by neither the first hand 112 nor the second hand 116.

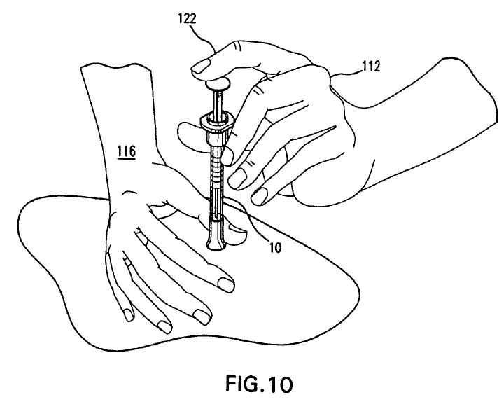

An additional variation has proven effective for administering the intradermal injection of the present invention. This variation includes gripping the syringe 60 with the same hand that is used to depress the plunger 66. FIG. 9 shows the syringe 60 being gripped with the first hand 112 while the plunger is simultaneously depressed with the thumb 120 of the first hand 112. This variation includes stretching the skin with the second hand 114 while the injection is being made. Alternatively, as shown in FIG. 10, the grip is reversed and the plunger is depressed by the forefinger 122 of the first hand 112 while the skin is being stretched by the second hand 116. However, it is believed that this manual stretching of the skin is unnecessary and merely represents a variation out of habit from using the standard technique.

In each of the variations described above, the needle cannula 24 is inserted only about 1.5 mm into the skin of the animal. Subsequent to administering the injection, the needle cannula 24 is withdrawn from the skin and the syringe 60 and needle assembly 20 are disposed of in an appropriate manner. Each of the variations were utilized in clinical trials to determine the effectiveness of both the needle assembly 20 and the present method of administering the intradermal injection.

In a specific embodiment the invention encompasses a method of making an injection into the skin of an animal comprising the following steps: (1) providing a drug delivery device, which includes a needle cannula having a forward needle tip such that the needle cannula is in fluid communication with a substance contained in the drug delivery device and includes a limiter portion surrounding the needle cannula and the limiter portion includes a skin engaging surface, so that the needle tip of the needle cannula extends from the limiter portion beyond the skin engaging surface a distance equal to approximately 0.5 mm to approximately 3.0 mm and the needle cannula has a fixed angle of orientation relative to a plane of the skin engaging surface of the limiter portion; (2) inserting the needle tip into the skin of an animal and engaging the surface of the skin with the skin engaging surface of the limiter portion such that the skin engaging surface of the limiter portion limits penetration of the needle tip into the dermis layer of the skin of the animal; and (3) expelling the substance from the drug delivery device through the needle tip into the skin of the animal. In some embodiments, the angle of the orientation of the needle cannula relative to a plane of the skin engaging surface is 90 degrees.

5.3 Substances

The present invention encompasses the administration of a wide variety of substances by selectively targeting them into a subject's skin with enhanced efficacy and safety profiles. Examples of substances that may be administered using the method of the present invention include, but are not limited to, pharmaceutically or biologically active substances including diagnostic agents, drugs, and other substances which provide therapeutic or health benefits, such as, but not limited to, neutraceuticals. The invention encompasses the administration of any protein, particularly a therapeutic protein, and all salts, polymorphs, analogs, derivatives, fragments, mimetics and peptides thereof, which can be obtained using standard methods known to one skilled in the art.

The principles of the invention may be analogously applied to compositions of one or more substances regardless of their viscosity, ionic compositions, size, hydrophobicity/hydrophilicity.

The form of the composition to be delivered or administered include solutions thereof in pharmaceutically acceptable diluents or solvents, emulsions, suspensions, gels, particulates such as micro- and nanoparticles either suspended or dispersed, as well as in-situ forming vehicles of the same. The compositions of the invention may be in any form suitable for delivery to the skin. In one embodiment, the dermal composition of the invention is in the form of a flowable, injectible medium, i.e., a low viscosity composition that may be injected in a syringe or pen. The flowable injectible medium may be a liquid. Alternatively the flowable injectible medium is a liquid in which particulate material is suspended, such that the medium retains its fluidity to be injectible and syringable, e.g., can be administered in a syringe.

The invention also includes compositions comprising particle reagents for diagnostic and/or therapeutic use and methods of delivery thereof. In brief, particles of defined shape and surface characteristics may be suspended in liquid media and delivered for example through micro needles to the selected compartment of the skin. Particle migration rate may be contingent on size and surface charge. As used herein, the term “particles” includes any formed element comprising monomers, polymers, lipids, amphiphiles, fatty acids, steroids, proteins, and other materials known to aggregate, self-assemble or which can be processed into particles. Particles also include unilamelar, multilamelar, random tortuous path and solid morphologies including but not limited to liposomes, microcrystalline materials, particulate MRI contrast agents, polymeric beads (i.e., latex and HEMA), but most preferably hollow particles, such as microbubbles, which are particularly useful for ultrasonic imaging.

The invention encompasses administration of compositions comprising one or more substances as disclosed herein in accordance with the methods of the invention. In some embodiments, the compositions of the invention comprise an effective amount of a substance e.g., a biologically active substance and one or more other additives. Additives that may be used in the compositions of the invention include for example, wetting agents, emulsifying agents, or pH buffering agents. The compositions of the invention may contain one or more other excipients such as saccharides and polyols. Additional examples of pharmaceutically acceptable carriers, diluents, and other excipients are provided in Remington's Pharmaceutical Sciences (Mack Pub. Co. N.J. current edition, all of which is incorporated herein by reference in its entirety.

The invention encompasses compositions in which the substance is in a particulate form, i.e., is not fully dissolved in solution. In some embodiments, at least 30%, at least 50%, at least 75% of the substance is in particulate form.

The invention encompasses the administration of any biologically active substance including without limitation, immunoglobulins (e.g., Multi-specific Igs, Single chain Igs, Ig fragments), Proteins, Peptides (e.g., Peptide receptors, PNAs, Selectins, binding proteins (maltose binding protein, glucose binding protein)), Nucleotides, Nucleic Acids (e.g., PNAS, RNAs, modified RNA/DNA, aptamers), Receptors (e.g., Acetylcholine receptor), Enzymes (e.g., Glucose Oxidase, HIV Protease and reverse transcriptase), Carbohydrates (e.g., NCAMs, Sialic acids), Cells (e.g., Insulin & Glucose responsive cells), bacteriophags (e.g., filamentous phage), viruses (e.g., HIV), Chemospecific agents (e.g., Cyptands, Crown ethers, Boronates).

Diagnostic substances useful with the present invention include macromolecular substances such as, for example, insulin, ACTH (e.g., corticotropin injection), luteinizing hormone-releasing hormone (e.g., Gonadorelin Hydrochloride), growth hormone-releasing hormone (e.g., Sermorelin Acetate), cholecystokinin (Sincalide), parathyroid hormone and fragments thereof (e.g. Teriparatide Acetate), thyroid releasing hormone and analogs thereof (e.g., protirelin), secretin and the like.

Therapeutic substances which can be used with the present invention include Alpha-1 anti-trypsin, Anti-Angiogenesis agents, Antisense, butorphanol, Calcitonin and analogs, Ceredase, COX-II inhibitors, dermatological agents, dihydroergotamine, Dopamine agonists and antagonists, Enkephalins and other opioid peptides, Epidermal growth factors, Erythropoietin and analogs, Follicle stimulating hormone, G-CSF, Glucagon, GM-CSF, granisetron, Growth hormone and analogs (including growth hormone releasing hormone), Growth hormone antagonists, Hirudin and Hirudin analogs such as Hirulog, IgE suppressors, Insulin, insulinotropin and analogs, Insulin-like growth factors, Interferons, Interleukins, Luteinizing hormone, Luteinizing hormone releasing hormone and analogs, Heparins, Low molecular weight heparins and other natural, modified, or synthetic glycoaminoglycans, M-CSF, metoclopramide, Midazolam, Monoclonal antibodies, Pegylated antibodies, Pegylated proteins or any proteins modified with hydrophilic or hydrophobic polymers or additional functional groups, Fusion proteins, Single chain antibody fragments or the same with any combination of attached proteins, macromolecules, or additional functional groups thereof, Narcotic analgesics, nicotine, Non-steroid anti-inflammatory agents, Oligosaccharides, ondansetron, Parathyroid hormone and analogs, Parathyroid hormone antagonists, Prostaglandin antagonists, Prostaglandins, Recombinant soluble receptors, scopolamine, Serotonin agonists and antagonists, Sildenafil, Terbutaline, Thrombolytics, Tissue plasminogen activators, TNF, and TNF antagonist, the vaccines, with or without carriers/adjuvants, including prophylactics and therapeutic antigens (including but not limited to subunit protein, peptide and polysaccharide, polysaccharide conjugates, toxoids, genetic based vaccines, live attenuated, reassortant, inactivated, whole cells, viral and bacterial vectors) in connection with, addiction, arthritis, cholera, cocaine addiction, diphtheria, tetanus, HIB, Lyme disease, meningococcus, measles, mumps, rubella, varicella, yellow fever, Respiratory syncytial virus, tick borne japanese encephalitis, pneumococcus, streptococcus, typhoid, influenza, hepatitis, including hepatitis A, B, C and E, otitis media, rabies, polio, HIV, parainfluenza, rotavirus, Epstein Barr Virus, CMV, chlamydia, non-typeable haemophilus, moraxella catarrhalis, human papilloma virus, tuberculosis including BCG, gonorrhoea, asthma, atheroschlerosis malaria, E-coli, Alzheimer's Disease, H. Pylori, salmonella, diabetes, cancer, herpes simplex, human papilloma and the like other substances including all of the major. therapeutics such as agents for the common cold, Anti-addiction, anti-allergy, anti-emetics, anti-obesity, antiosteoporeteic, anti-infectives, analgesics, anesthetics, anorexics, antiarthritics, antiasthmatic agents, anticonvulsants, antidepressants, antidiabetic agents, antihistamines, anti-inflammatory agents, antimigraine preparations, antimotion sickness preparations, antinauseants, antineoplastics, antiparkinsonism drugs, antipruritics, antipsychotics, antipyretics, anticholinergics, benzodiazepine antagonists, vasodilators, including general, coronary, peripheral and cerebral, bone stimulating agents, central nervous system stimulants, hormones, hypnotics, immunosuppressives, muscle relaxants, parasympatholytics, parasympathomimetrics, prostaglandins, proteins, peptides, polypeptides and other macromolecules, psychostimulants, sedatives, and sexual hypofunction and tranquilizers.

Other substances that are particularly suited for the methods of the invention are which can benefit from a reduced risk of unwanted immune response and immuno-toxic effects and those which can benefit from an improved pharmacokinetic profile, including but not limited to low molecular weight heparins, pentasaccharides, interferon alpha and beta, erythropoeitines, antibodies, polypeptidic hormones, growth hormone, and interleukins.

The invention encompasses administration of therapeutic antibodies in accordance with the methods of the invention which include but are not limited to HERCEPTIN® (Trastuzumab) (Genentech, Calif.) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXINT™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); ICM3 is a humanized anti-ICAM3 antibody (ICOS Pharm); IDEC-114 is a primatied anti-CD80 antibody (IDEC Pharm/Mitsubishi); ZEVALIN™ is a radiolabelled niurine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech).

The invention encompasses administration of chemotherapeutic agents, radiation therapeutic agents, hormonal therapeutic agents, immunotherapeutic agents, immunomodulatory agents, anti-inflammatory agents, antibiotics, anti-viral agents, and cytotoxic agents.

Non-limiting examples of anti-inflammatory agents include non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxygenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

Examples of immunomodulatory agents include, but are not limited to, methothrexate, ENBREL, REMICADET™, leflunomide, cyclophosphamide, cyclosporine A, and macrolide antibiotics (e.g., FK506 (tacrolimus)), methylprednisolone (MP), corticosteroids, steriods, mycophenolate mofetil, rapamycin (sirolimus), mizoribine, deoxyspergualin, brequinar, malononitriloamindes (e.g., leflunamide), T cell receptor modulators, and cytokine receptor modulators, corticosteroids, cytokine agonists, cytokine antagonists, and cytokine inhibitors.

Examples of antibiotics include, but are not limited to, macrolide (e.g., tobramycin (Tobi®)), a cephalosporin (e.g., cephalexin (Keflex®), cephradine (Velosef®)), cefuroxime (Ceftin®), cefprozil (Cefzil®), cefaclor (Ceclor®), cefixime (Suprax®) or cefadroxil (Duricef®)), a clarithromycin (e.g., clarithromycin (Biaxin®)), an erythromycin (e.g., erythromycin (EMycin®)), a penicillin (e.g., penicillin V (V-Cillin K® or Pen Vee K®)) or a quinolone (e.g., ofloxacin (Floxin®), ciprofloxacin (Cipro®) or norfloxacin (Noroxin®)), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefminox), monobactarns (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin, chloramphenicols, erythromycin, penicillin, streptomycin, vancomycin, trimethoprimsulfamethoxazols, and tuberin.

Examples of anti-viral agents include, but are not limited to, protease inhibitors, nucleoside reverse transcriptase inhibitors, non-nucleoside reverse transcriptase inhibitors and nucleoside analogs, zidovudine, acyclovir, gangcyclovir, vidarabine, idoxuridine, trifluridine, and ribavirin, as well as foscamet, amantadine, rimantadine, saquinavir, indinavir, amprenavir, lopinavir, ritonavir, the alpha-interferons; adefovir, clevadine, entecavir, and pleconaril

5.4 Determination of Efficacy of the Methods of the Invention

The efficacy, including therapeutic efficacy, of formulations containing a substance of the present invention may be determined using any standard method known to one skilled in the art or described herein. The assay for determining the efficacy of the formulations of the invention may be in vivo or in vitro based assays, including animal based assays. Preferably, the efficacy of the formulations of the invention is done in a clinical setting.

The efficacy of the delivery methods of the invention may be determined by assessing various factors including completeness of infusion, pressure and flow rate of delivery, safety of delivery as determined using monitoring adverse reactions at the injection site. The completeness of infusion may be determined, for example by measuring the amount of a solution delivered versus the amount of the solution which leaks out of the infusion site. A complete or successful infusion/injection is defined as less than or equal to 10% leakage of total fluid volume delivered as determined by gravimetric methodology. An exemplary gravimetric methodology for determining the leakage out of infusion site or failure of fluid to enter skin, immediately following each infusion may comprise the following: After removal of the device, a pre-weighed absorbent swab is placed against the skin and the device to collect any visible fluid that leaks out or does not penetrate the skin. The swab is re-weighed and the fluid volume is calculated.

The pressure of delivery may be monitored using any standard methods for monitoring fluid pressure as known to one skilled in the art. In a specific embodiment the pressure of delivery is monitored and recorded using a Becton Dickinson DTX Plus TNF-R blood pressure transducer approved for human use. The procedure may comprise the following: the transducer is plumbed into the infusion system via a four-way stopcock; the transducer is connected using a single cable to a WPI TBM4M power supply/signal conditioner, which in turn passes on the amplified signal to a Fluke Hydra Data Bucket. The Data Bucket converts, digitizes, and caches the data until it is retrieved by a PC for storage and data processing. Alternatively, instead of the Fluke Hydra Data Bucket, a PC-based A/D data acquisition card may be used to digitize the analog output from the WPI signal conditioner.

The safety of the delivery methods of the invention may be determined by assessing the development of any adverse skin effects at various times following infusion for example using the Draize scoring method. An exemplary draize scoring scale is as follows:

Draize Scoring Scale

| | |
|---|---|
| Erythema | Edema |

| | | | |
|---|---|---|---|
| No erythema | 0 | No edema | 0 |
| Very slight erythema | 1 | Very slight edema | 1 |
| Well defined erythema | 2 | Slight edema | 2 |
| Moderate to severe erythema | 3 | Moderate edema | 3 |
| Severe Erythema (beet-red to | 4 | Severe edema | 4 |
| eshar formation) | | | |

Draize scoring and assessment of any other cutaneous events are preferably done immediately following delivery of the substance.

The invention encompasses assessing pain perception in the subject using a Gracely Box SL Scale for Pain Intensity (scale of from 0 (no pain sensation) to 20 (extremely intense for 18 and up)) and the Gracely Pain Unpleasantness scale (scale of from 0 (neutral) to 20 (very intolerable for 17 and up)). Immediate sensory perception of the pain is rated by the subject at various times during infusion. The invention also encompasses recording pain and unpleasantness, i.e., the measure of how much a pain sensation bothers the subject, perceived by the subject at least twice during each treatment. An exemplary methods for monitoring and evaluating pain and unpleasantness may comprise the following: first, after the device has been applied, the subject is asked to rate the pain perceived at that moment following the needle stick; second, after the total dose has been infused the subject is asked to rate the overall perceived pain for the entire infusion process, including needle stick.

The methods of the invention also encompass evaluating wheal formation upon injection of a substance in accordance with the methods of the invention After an infusion device is removed from the skin, information about the wheal (e.g., presence of a wheal) is observed and recorded.

In some embodiments, the pharmacokinetic and pharmacodynamic parameters of the delivery of a substance of the invention is determined, preferably quantitatively using standard methods known to one skilled in the art. In preferred embodiments, the pharmacodynamic and pharmacokinetic properties of a substance of the invention, delivered using the methods of the invention, are compared to those of the substance delivered by other conventional modes of administration, e.g., subcutaneous or intramuscular delivery, to establish the therapeutic efficacy of the substance administered in accordance with the methods of the invention. Pharmacokinetic parameters that may be measured in accordance with the methods of the invention include but are not limited to Tₘₐₓ, Cₘₐₓ, T_{lag}, AUC, etc. Other pharmacokinetic parameters that may be measured in the methods of the invention include for example, half-life (tui), elimination rate constant and partial AUC values. Standard statistical tests which are known to one skilled in the art may be used for the statistical analysis of the pharmacokinetic and pharmacodynamic parameters obtained.

In a specific embodiment, the invention encompasses determining the therapeutic efficacy of a substance administered in accordance with the methods of the invention by comparing the pharmacokinetic profile to that of, for example, subcutaneous or intramuscular delivery.

### 6. EXAMPLES

6.1 Effects of Needle Length on Delivery of Substances

To investigate safety, performance, subject's perception of pain over time and back-pressure generated by tissue resistance during delivery of saline in the thigh using different lengths of needles, the following procedures were designed: A total of 10 subjects received a total of 4 treatment, alternating on the right and left thighs, with microneedle device prototypes using a randomized schedule according to the following parameters.

TABLE 1
Parameters of Treatments

| | | | | Rate | |
|---|---|---|---|---|---|
| Infusion | Device | Volume μl | Site | ~Total time | |
| 1 | 1 mm | 200 μL | thigh | 50 | μL/min |
| | | | | 4 | min |
| 2 | 2 mm | 200 μL | thigh | 50 | μL/min |
| | | | | 4 | min |
| 3 | 3 mm | 200 μL | thigh | 50 | μL/min |
| | | | | 4 | min |
| 4 | 1 mm | 100 μL | thigh | 100 | μL/min |
| | | | | 1 | min |

6.1.1 Test Materials and Supplies

6.1.1.1 Microneedle Device

The microneedle devices consist of single 34 gauge 1, 2 or 3 mm microneedle housed in a 1 inch diameter urethane catheter hub with an 18 inch section of polyurethane tubing as a flow path. An adhesive ring was applied to the device perimeter just prior to use.

6.1.1.2 Pressure DAQ System

Pressure was monitored and recorded using a Becton Dickinson DTX Plus TNF-R blood pressure transducer approved for human use. The transducer was plumbed into the infusion system via a four-way stopcock. The transducer was connected using a single cable to a WPI TBM4M power supply/signal conditioner, which in turn passed on the amplified signal to a Fluke Hydra Data Bucket. The Data Bucket converts, digitizes, and caches the data until it is retrieved by a PC for storage and data processing. Alternatively, instead of the Fluke Hydra Data Bucket, a PC-based A/D data acquisition card may be used to digitize the analog output from the WPI signal conditioner.

6.1.3 Failures

Three failures were observed, all of which occurred with the 1 mm/100 μl device. All data corresponding to these three failures were removed from further analyses.

6.1.4 Pressure

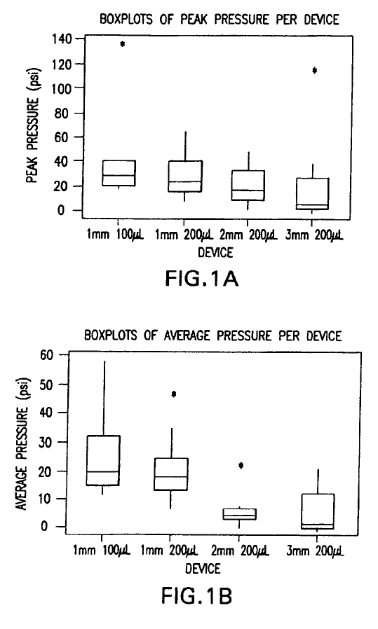

Pressure in the fluid path was measured via in line pressure transduction equipment. Peak pressure and sustaining (or average) pressure were recorded. All peak pressure and average pressure measurements per treatment combination are shown in FIG. 1. There were 2 outlying/unusual observations for the peak pressure (represented as stars in FIG. 1) and 2 outlying/unusual observations for the average pressure.

Summary statistics of the peak pressure and average pressure measurements per treatment combination are shown below in Table 2.

TABLE 2
Summary Statistics

| | | Device &Volume | | | |
|---|---|---|---|---|---|
| | | 1 mm/100 μl | 1 mm/200 μl | 2 mm/200 μl | 3 mm/200 μl |
| | | n = 7 | n = 10 | n = 10 | n = 10 |
| Peak | Mean | 44.3 | 29.97 | 21.21 | 20.8 |
| Pressure | Median | 28.3 | 23.95 | 17.30 | 6.6 |
| | Standard Deviation | 41 | 18.89 | 15.85 | 35 |
| | Min | 19.3 | 8.4 | 1.3 | 0.7 |
| | Max | 135.5 | 64.4 | 48.5 | 114.7 |
| Average | Mean | 25.7 | 20.88 | 6.51 | 6.26 |
| Pressure | Median | 19.6 | 18.40 | 5 | 2.40 |
| | Standard Deviation | 15.96 | 11.89 | 6.05 | 8.22 |
| | Min | 11.8 | 7 | 0.6 | 0.0 |
| | Max | 58.6 | 47.1 | 22.6 | 21.5 |

6.1.4.1 Difference between Peak and Average Pressure within Treatment

Because of the non-normality and outliers in the data, a non-parametric test (Wilcoxon's signed rank test) was used to compare peak and average pressure within each treatment combination. The results showed that the median peak pressure was significantly higher than the median average pressure for all treatment combinations.

6.1.4.2 Treatment Effect on Peak and Average Pressure

An analysis of variance (“ANOVA”) was used on the peak and average measurements to determine whether the treatment combination had any significant effect. The ANOVA model included a subject effect, an injection number effect and a treatment combination effect. The results indicated that there is no significant device effect for mean peak pressure. (with the observed variability in peak pressure and a sample size of 10, there was a 90% power to detect a difference of about 45 psi in peak pressure between any two treatments combination).

There was a significant device effect on the mean average pressure. Multiple comparisons indicated that the significant differences are between the 1 mm devices (both 100 μl and 200 μl) and the other two devices. In particular, the mean average pressure for the 1 mm/200 μL was significantly larger by 15.1 psi than the mean average pressure for the 2 mm/200 μl (with 95% confidence interval for the mean difference of (1.3, 28.8)). The mean average pressure for the 1 mm/200 μl was significantly larger by 14.9 psi than the mean average pressure for the 3 mm/200 μl (with 95% confidence interval for the mean difference of (1.1, 28.7)).

As part of the ANOVA procedure, a test for equality of variance was performed and there was no significant device effect on variability for either the peak or average pressures.

6.1.5 Pain

Table 3 summarizes the statistics of the needlestick pain and end of injection pain per treatment combination.

TABLE 3
Summary of Pain

| | | Device &Volume | | | |
|---|---|---|---|---|---|
| | | | | 2 mm/ | 3 mm/ |
| | | 1 mm/100 μl | 1 mm/200 μl | 200 μl | 200 μl |
| | | n = 7 | n = 10 | n = 10 | n = 10 |
| Needle stick | Mean | 1.4 | 1.9 | 2.1 | 1.5 |
| | Median | 1 | 1 | 1 | 1 |
| | Std. Dev. | 0.5 | 2.1 | 2.5 | 1 |
| | Min | 1 | 1 | 1 | 1 |
| | Max | 2 | 8 | 9 | 4 |
| End of | Mean | 9 | 8.4 | 2.3 | 2.9 |
| Injection | Median | 8 | 8.5 | 1.5 | 1.5 |
| | Std. Dev. | 4.4 | 3.9 | 1.8 | 3 |
| | Min | 3 | 2 | 1 | 1 |
| | Max | 16 | 14 | 6 | 10 |

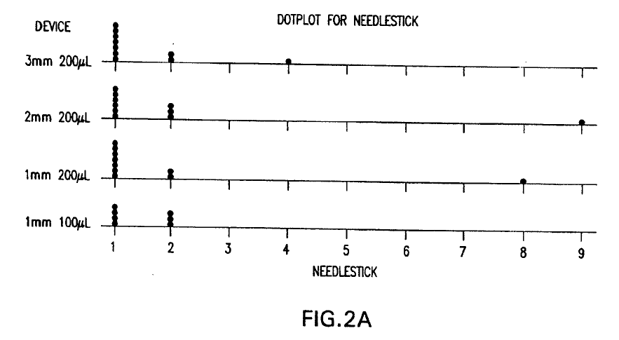

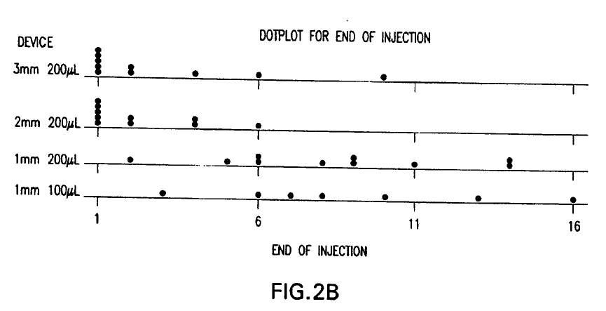

The distribution of pain scores is shown in FIG. 2.

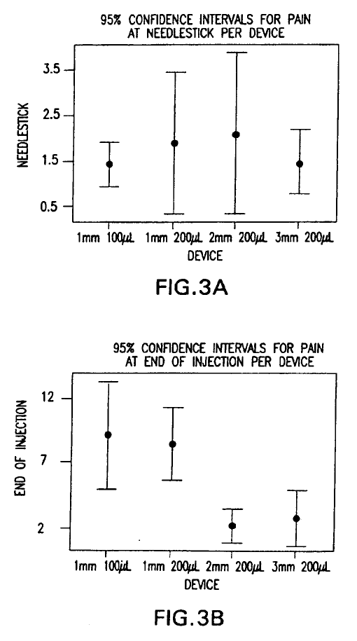

The Pain values were analyzed using ANOVA. The ANOVA models included effects of subject-to-subject differences, order of injection, time (needlestick or end), device, time by device interaction and the peak pressure or average pressure as a covariate. The ANOVA results showed that there is a significant time effect (p-value<0.0005), device effect (p-value<0.005) and time by device interaction (p-value<0.0005). The peak or average pressures were not significant covariates. Multiple comparisons indicated that significant differences in pain exist at the end of the injection. In particular, at the end of injection, both 1 mm devices had significantly higher pain on average than the 2 mm/200 μl and 3 mm/200 μl devices. The following results were observed: 1) the mean pain at the end of injection for the 1 mm/200 μl was significantly higher by 6.3 than the mean pain at the end of injection for the 2 mm/200 μl (with 95% confidence interval for the mean difference in pain of (3, 9.7)); and 2) the mean volume at initial sensation for the 1 mm/200 μl was significantly higher by 5.7 than the mean pain at the end of injection for the 3 mm/200 μl (with 95% confidence interval for the mean difference in pain of (2.4, 9.1)). FIG. 3 shows confidence intervals for pain at each time point per device.

6.1.6 Wheal

Table 4 summarizes the number of wheals for each device given the injection was successful. A chi-squared test of homogeneity indicated a significant difference in probability of wheal formation for the various devices. The 1 mm devices are significantly more likely to results in a wheal than the other two devices.

TABLE 4
Wheal Formation

| | |
|---|---|
| Injection Type | Wheals |

| Device | Volume | Absent | Present |
|---|---|---|---|
| 1 mm | 100 | 0 | 7 (100%) |
| 1 mm | 200 | 0 | 10 (100%) |
| 2 mm | 200 | 9 (90%) | 1 (10%) |
| 3 mm | 200 | 10 (100%) | 0 |

6.1.7 Leakage

Table 5 summarizes the number of time leakage was observed for each device and where the leakage was observed given the injection was successful. A chi-squared test of homogeneity indicates a significant difference in probability of leakage for the various devices. There is no significant difference in the average volume of fluid collected for the various devices.

TABLE 5
Leakage

| | | Leakage | | | | Fluid Collected | |
|---|---|---|---|---|---|---|---|
| Injection Type | | | De- | | | | Average |
| Device | Volume | Yes | vice | Skin | both | Yes | Volume |
| 1 mm | 100 | 7 (100%) | 5 | 0 | 2 | 7 (100%) | 1.66 |
| 1 mm | 200 | 8 (80%) | 8 | 0 | 0 | 8 (80%) | 1.14 |
| 2 mm | 200 | 4 (40%) | 2 | 1 | 1 | 4 (40%) | 0.8 |
| 3 mm | 200 | 3 (30%) | 1 | 0 | 2 | 3 (30%) | 0.75 |

6.1.8 Erythema and Edema

Table 6 summarizes the erythema and edema Draize scores assessed at the end of the study. There was no significant difference in erythema between the injection types. There was a significant difference in edema scores between the injection types, in particular, the 3 mm/200 μl device had significantly lower edema scores than both 1 mm devices.

TABLE 6
Erythema and Edema Draize Scores

| | | |
|---|---|---|
| Injection Type | Edema | Erythema |

| Device | Volume | 0 | 1 | 2 | 3 | 0 | 1 |
|---|---|---|---|---|---|---|---|
| 1 mm | 100 | 1 | 2 | 4 | 0 | 5 | 2 |
| 1 mm | 200 | 4 | 3 | 2 | 1 | 6 | 4 |
| 2 mm | 200 | 7 | 3 | 0 | 0 | 5 | 5 |
| 3 mm | 200 | 9 | 1 | 0 | 0 | 7 | 3 |

6.1.9 Other Performance Factors

Except for the failures, all devices were able to administer all fluid. In addition, all devices were considered easy to apply.

6.2 Constant Pressure Infusion Using a Modified Constant Rate Pump

To investigate the methodology for a constant pressure infusion based on a modified constant rate pump, and the effects of needle length, number of needles and pressure on various characteristics of fluid infusion, studies were performed using the following procedures: A total of 20 subjects received up to 7 infusions or injections of saline in anterior thigh with investigational microneedle prototypes described below in Table 7, using the parameters summarized in the same table. Infusions were performed in alternating thighs, with three or four infusions per thigh. Prior to the study start date, the infusion sequence was randomized for each subject.

TABLE 7
Parameters for Infusions

| | | | | Holding time Prior to | |
|---|---|---|---|---|---|
| | | | | removing | Total time |
| Treatment | Device | PSI | Time/Volume | device from skin | (Max. time) |
| 1 | 1 needle × | 15 | 5 minutes or | 2 minutes | Up to 7 |
| | 1 mm | | 250 ul total | | minutes |
| | | | volume delivered | | |
| 2 | 1 needle × | 20 | 5 minutes or | 2 minutes | Up to 7 |
| | 1 mm | | 250 ul total | | minutes |
| | | | volume delivered | | |
| 3 | 1 needle × | 15 | 5 minutes or | 2 minutes | Up to 7 |
| | 1.5 mm | | 250 ul total | | minutes |
| | | | volume delivered | | |
| 4 | 1 needle × | 20 | 5 minutes or | 2 minutes | Up to 7 |
| | 1.5 mm | | 250 ul total | | minutes |
| | | | volume delivered | | |
| 5 | 1 needle × | 15 | 5 minutes or | 2 minutes | Up to 7 |
| | 2 mm | | 250 ul total | | minutes |
| | | | volume delivered | | |
| 6 | 1 needle × | 20 | 5 minutes or | 2 minutes | Up to 7 |
| | 2 mm | | 250 ul total | | minutes |
| | | | volume delivered | | |
| 7 | 3 needle × | 15 | 5 minutes or | 2 minutes | Up to 7 |
| | 1 mm | | 250 ul total | | minutes |
| | | | volume delivered | | |

The infusion pressure and rate were controlled by a Harvard syringe pump modified to receive pressure feedback during infusion, and controlled using PID algorithm software which utilizes real-time feedback to adjust flow. Maximum rate (1000 μl/min), volume (250 uL), and flow duration (5 min) were all set as controlled factors within the PID algorithm to ensure safety by preventing runaway pump infusion. Real time pressure and flow profiles were measured electronically during infusion. These data were analyzed post-infusion, to gather information about the pressure/rate/needle-depth flow relationships.

There were two failures (1 needle, 1 mm, 15 psi and 3 needles, 1 mm, 15 psi), but these failures were not sufficient for a significant difference between the devices. All data corresponding to these two failures were removed from the analyses.

6.2.1 Instrumentation for Constant Pressure Delivery

The Virtual Instrument (“VI”) that collects and records pressure and flow data also controls pressure. A PC-based system receives a signal from a pressure transducer, processes that signal through a Proportional-Integral-Derivative (PID) algorithm and sends a control signal to a syringe pump. The syringe pump drives a 1 cc syringe connected to the infusion device and pressure transducer. This provides a closed-loop control of pressure by modulation of pump speed.

For closed-loop control a LabVIEW advanced PID control module is installed in the VI. The PID module is implemented using gain scheduling to achieve reasonable startup times under a wide range of flow conditions. The PID module controls the pressure through an iterative process. Pressure, change in pressure, and current flow rate are reviewed by the VI. PID values in the gain schedule are applied to an algorithm to calculate the next flow rate setting. Flow rate (pumping speed) is updated three times per second to maintain infusate pressure at or near set point.

Pressure is sensed with a BD DTX Plus TNF-R pressure transducer installed in the infusate flow path. The transducer signal is transferred to the PC via the NI A/D card. A Harvard PHD2000 syringe pump is used to deliver infusate at a controlled rate. The PHD2000 is controlled by the VI from the PC through an RS-232 serial communication link. Both flow rate and final delivered volume are set in the pump by the VI.

The VI is built with a number of process variables that can be set by the operator. To provide reproducibility these variables are preconfigured based on settings optimized for needle penetration length and pressure set point. Those process variables are stored in configuration files that are preloaded prior to each infusion. Parameters include set point pressure, maximum infusion volume, maximum infusion time, maximum flow rate, syringe diameter and start delay.

6.2.2 Statistical Analysis

A Bonferroni correction was applied to the alpha-level to account for separate tests being performed. In order to have an overall alpha of 0.05, p-values less than 0.025 were considered significant.

Treatments 1-6 (single needle devices) form a 32 factorial design with needle length (3 levels) and set pressure (2 levels). For the single needle devices, fluid flow rate (peak and average) was compared using ANOVA. The ANOVA models included subject-to-subject differences, order of injection, needle lengths, set pressure and the needle lengths by set pressure interaction. Post-hoc multiple comparisons were performed if the factor effects were significant. The post-hoc comparisons helped identify which factor levels actually differ from each other. To determine the effect of number of needles, treatments 1 (1 needle, 1 mm, 15 psi) & 7 (3 needle, 1 mm, 15 psi) were compared with a paired t-test.

Pain scale scores and completeness of injection/infusion (calculated fluid volumes) were analyzed using the same protocol. Binary responses were summarized per needle length, set pressure and treatment. These responses were analyzed using Fisher's exact test or a binary logistic regression. Responses using 0-3 or 0-4 scales (Draize scores, bleeding) were summarized per needle length, set pressure and treatment. These responses were analyzed via Chi-Squared tests of homogeneity or ordinal logistic regression.

6.2.3 Pressure and Flow Rate

Summary statistics of the pressure and flow rate per treatment combination are shown in Table 8 below. The standard deviations in the table represent the total variability and contain a between donor component.

Below are the definitions for the terms represented in Table 8:

“t1” refers to the time when flow into tissue begins; “p1” refers to the pressure at time t1; “fr1” refers to the flow rate at time t1; “t2” refers to the time at the start of the steady state; “t3” refers to the time at the finish of the steady state; “avgp” refers to the average pressure during the steady state; :minfrp” refers to the minimum flow rate during the steady state divided by the average pressure; “maxfrp” refers to the maximum flow rate during the steady state divided by the average pressure; and “avgfrp” refers to the average flow rate during the steady state divided by the average pressure. Steady state refers to period of stable pressure during injection.

TABLE 8
Summary Statistics

| | | Device | | | | | | |
|---|---|---|---|---|---|---|---|---|
| | | 1 needle | | | | | | 3 needle |
| | | 1 mm | | 1.5 mm | | 2.00 mm | | 1 mm |
| | | 15 psi | 20 psi | 15 psi | 20 psi | 15 psi | 20 psi | 15 psi |
| t1 | Mean | 14.47 | 15.81 | 11.12 | 11.04 | 9.38 | 10.49 | 19.21 |
| (sec) | SD | 9.09 | 9.33 | 3.68 | 3.68 | 3.55 | 3.33 | 8.09 |
| | Min | 7.8 | 7.4 | 6.4 | 6.6 | 6.4 | 6.4 | 8 |
| | Max | 41.8 | 41.4 | 20.8 | 21.2 | 18.6 | 17.8 | 39.4 |
| p1 | Mean | 5.34 | 8.84 | 4.97 | 5.15 | 4.94 | 5.8 | 5.62 |
| (psi) | SD | 2.36 | 4.19 | 3.42 | 3.34 | 3.18 | 3.93 | 4.02 |
| | Min | 1.9 | 1.5 | 1.1 | 1.6 | 0.6 | 1.3 | 1.4 |
| | Max | 10.1 | 18 | 14 | 12.8 | 10.9 | 15.1 | 15.1 |
| fr1 | Mean | 40.88 | 55.52 | 39.58 | 54.47 | 184 | 193.51 | 62.67 |
| (μL/min) | SD | 15.21 | 21.98 | 16.62 | 20.33 | 136.61 | 147.22 | 61.9 |
| | Min | 10.6 | 16.4 | 0.1 | 14.2 | 13.5 | 17.5 | 13.6 |
| | Max | 77.2 | 129.5 | 60 | 80 | 593.3 | 521.6 | 293.5 |
| t2 | Mean | 38.23 | 42.1 | 42.47 | 39.25 | 27.58 | 32.76 | 42.89 |
| (sec) | SD | 15.24 | 16.22 | 27.17 | 10.34 | 12.04 | 11.8 | 12.34 |
| | Min | 22.6 | 21.2 | 23.4 | 26.8 | 19.2 | 19.2 | 29.6 |
| | Max | 79 | 93.2 | 147.6 | 64.9 | 64 | 64 | 82 |
| t3 | Mean | 82.09 | 88.14 | 58.18 | 51.26 | 39.62 | 39.77 | 67.45 |
| (sec) | SD | 29.52 | 44.76 | 26.5 | 8.65 | 11.98 | 11.01 | 23.93 |
| | Min | 45.2 | 47.4 | 43 | 39.2 | 32.8 | 32.2 | 39.6 |
| | Max | 142 | 196.4 | 163.4 | 71.6 | 75.2 | 70.6 | 128.6 |
| avgp | Mean | 14.85 | 19.19 | 13.86 | 17.36 | 13.69 | 17.62 | 13.66 |
| (psi) | SD | 0.94 | 1.29 | 0.56 | 1.91 | 0.65 | 1.58 | 0.66 |
| | Min | 13.1 | 14.4 | 12.6 | 11.8 | 11.3 | 11.8 | 12.1 |
| | Max | 16.6 | 20.7 | 14.7 | 19.4 | 14.4 | 19.8 | 15.1 |
| maxp | Mean | 16.18 | 20.59 | 15.16 | 18.91 | 14.83 | 19.02 | 14.64 |
| (psi) | SD | 1.46 | 1.49 | 0.76 | 1.52 | 0.46 | 1.5 | 0.7 |
| | Min | 14.3 | 16.1 | 14.1 | 14.7 | 13.8 | 14.6 | 13.4 |
| | Max | 19.8 | 22.8 | 17 | 21.1 | 15.7 | 22.4 | 15.7 |
| minp | Mean | 13.68 | 17.71 | 12.4 | 15.59 | 12.34 | 15.67 | 12.6 |
| (psi) | SD | 0.94 | 1.42 | 0.96 | 2.33 | 1.01 | 2.04 | 0.92 |
| | Min | 11.8 | 12.5 | 9.8 | 9.3 | 9.1 | 9.4 | 10.6 |
| | Max | 15.5 | 19.6 | 13.5 | 18.1 | 13.5 | 18.8 | 14.5 |
| maxfr | Mean | 325.85 | 364.51 | 587.08 | 722.66 | 713.71 | 856.18 | 564.8 |
| (μL/min) | SD | 128.33 | 207.17 | 128.14 | 161.42 | 97.69 | 153.08 | 232.41 |
| | Min | 137.3 | 97.4 | 330.7 | 429.4 | 480.6 | 446.4 | 153.7 |
| | Max | 625.5 | 1000 | 804.2 | 994.3 | 866.1 | 1000 | 1000 |
| minfr | Mean | 278.4 | 321.23 | 531.38 | 628.54 | 656.14 | 799.15 | 478.04 |
| (μL/min) | SD | 124.13 | 202.31 | 134.84 | 149.91 | 91.42 | 149.53 | 248.6 |
| | Min | 98.2 | 62.8 | 245.4 | 321.8 | 454.5 | 444.1 | 131.8 |
| | Max | 524.1 | 855.6 | 766.3 | 921 | 777.9 | 978.7 | 975 |
| avgfr | Mean | 304.11 | 345.24 | 563.46 | 682.38 | 690.1 | 831.52 | 522.09 |
| (μL/min) | SD | 127.98 | 204.31 | 130.91 | 154.31 | 94.5 | 152.12 | 238.02 |
| | Min | 115.5 | 87.2 | 294.2 | 425.5 | 471.6 | 445.1 | 145.3 |
| | Max | 562.4 | 945 | 786.5 | 974.6 | 846.5 | 992 | 996 |
| minfrp | Mean | 18.91 | 17.32 | 38.37 | 36.89 | 48.08 | 45.72 | 35.58 |
| (μL/min- | SD | 8.79 | 12.94 | 9.82 | 10.49 | 7.07 | 9.05 | 19.68 |
| psi) | Min | 6.5 | 3.3 | 17.4 | 18 | 31.6 | 22.5 | 9.1 |
| | Max | 36.8 | 59.6 | 55.7 | 55.2 | 57.3 | 56.8 | 75.7 |
| maxfrp | Mean | 22.17 | 19.73 | 42.46 | 42.64 | 52.36 | 49.09 | 42 |
| (μL/min- | SD | 9.44 | 13.97 | 9.68 | 12.6 | 7.94 | 9.71 | 18.85 |
| psi) | Min | 9.2 | 5.1 | 23.4 | 22.2 | 33.4 | 22.6 | 10.6 |
| | Max | 47.6 | 69.7 | 58.9 | 68.1 | 61.5 | 58.4 | 77.6 |
| avgfrp | Mean | 20.68 | 18.66 | 40.74 | 40.15 | 50.58 | 47.6 | 38.84 |
| (μL/min- | SD | 9.23 | 13.53 | 9.75 | 11.51 | 7.51 | 9.38 | 19.1 |
| psi) | Min | 7.7 | 4.3 | 20.8 | 22 | 32.8 | 22.5 | 10.1 |
| | Max | 42.8 | 65.8 | 57.4 | 59.7 | 60.1 | 57.5 | 77.3 |

Treatments 1-6 (single needle devices) form a 3×2 factorial design with the factors: needle length (3 levels) and set pressure (2 levels). For the single needle devices, p1 (pressure at time t1), flow rates (minfr, maxfr and avgfr) and normalized flow rate (minfrp, maxfrp and avgfrp) were compared using ANOVA. The ANOVA models included subject-to-subject differences, order of injection, leg (R or L), needle lengths, set pressure and the needle lengths by set pressure interaction. Post-hoc multiple comparisons were performed if the factor effects are significant. The post-hoc comparisons helped identify which factor levels actually differ from each other. To determine the effect of number of needles, treatments 1 (1 needle×1 mm, 15 psi) & 7 (3 needle×1 mm, 15 psi) were compared with a paired t-test.

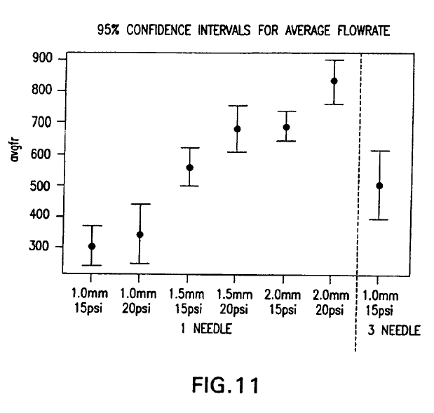

Individual 95% confidence intervals for the above responses for all treatments are shown in FIG. 11.

6.2.4 Pain

Pain was determined using a Gracely Box SL Scale. Pain scores were recorded at the time of the needlestick and for the process. Summary statistics of the needlestick pain and end of injection pain per treatment combination are shown in Table 9 below.

TABLE 9
Summary Statistics

| | | Device | | | | | | |
|---|---|---|---|---|---|---|---|---|
| | | 1 needle | | | | | | 3 needle |
| | | 1 mm | | 1.5 mm | | 2.00 mm | | 1 mm |
| | | 15 psi | 20 psi | 15 psi | 20 psi | 15 psi | 20 psi | 15 psi |
| Needle | Mean | 0.5 | 0.5 | 0.8 | 0.9 | 1 | 1.15 | 0.8 |
| Stick | Median | 0 | 0 | 0 | 1 | 0 | 1 | 0 |
| | SD | 1.2 | 0.8 | 1.5 | 1.3 | 2.4 | 1.8 | 1.3 |
| | Min | 0 | 0 | 0 | 0 | 0 | 0 | 0 |
| | Max | 5 | 3 | 5 | 5 | 10 | 7 | 4 |
| Process | Mean | 7.1 | 7.4 | 4.2 | 5.1 | 3.6 | 4.5 | 8.9 |
| | Median | 7 | 7 | 4 | 5 | 3 | 4 | 8 |
| | SD | 4 | 3.3 | 3.5 | 3.3 | 3.4 | 3.9 | 3.8 |
| | Min | 1 | 1 | 0 | 0 | 0 | 0 | 3 |
| | Max | 16 | 13 | 13 | 13 | 11 | 11 | 17 |

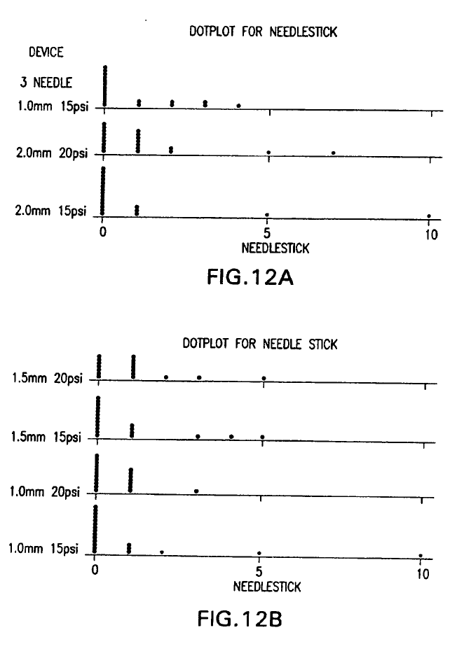

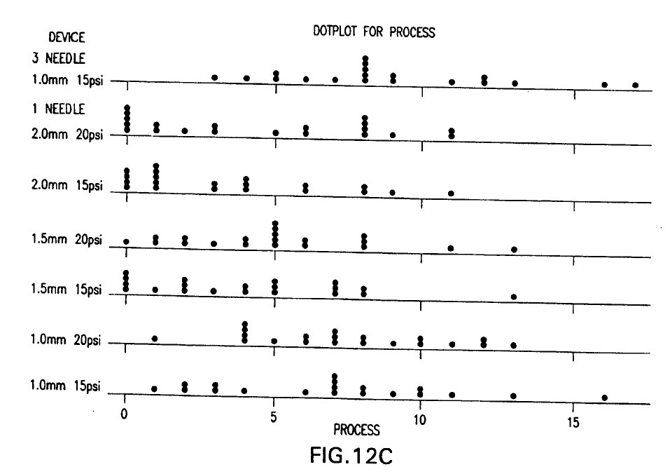

The distribution of pain scores are shown in FIG. 12. Treatments 1-6 (single needle devices) form a 3×2 factorial design with needle length (3 levels) and set pressure (2 levels). For the single needle devices, pain values (needle stick and process) were compared using ANOVA. The ANOVA models included subject-to-subject differences, order of injection, Leg (R or L), needle lengths, set pressure and the needle lengths by set pressure interaction. The ANOVA results showed the following:

Needle Stick The ANOVA was performed on transformed data because of the non-normality in the responses. The only significant effect was the subject effect.

Process: There was a significant subject effect and needle length effect (p-value<0.0005). Multiple comparisons indicated that the mean pain for the 1 mm needles was significantly higher than mean pain for the 1.5 mm and 2 mm needles.

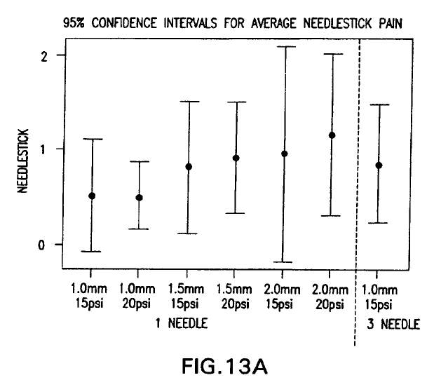

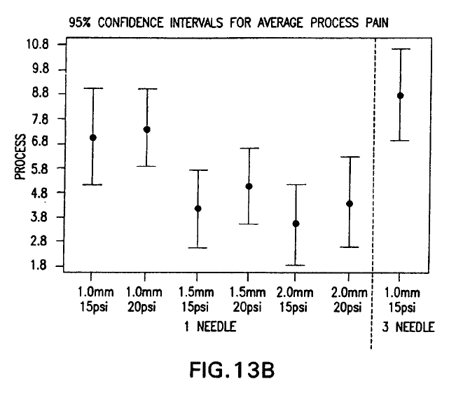

To determine the effect of number of needles, treatments 1 (1 needle×1 mm, 15 psi) & 7 (3 needle×1 mm, 15 psi) were compared with a paired t-test. No significant “number of needles” effect was observed for needle stick or process. With a sample size of 20 subjects, there was a 90% chance to detect a difference of about 1 or more unit of pain for the needle stick and 3 or more units of pain for the process pain. Individual confidence intervals for Needle Stick pain and Process pain per device are shown in FIG. 13.

6.2.5 Wheal

The number of wheals for each device is summarized in Table 10 below. The eight responses given as “Not Sure” were treated as missing data (there were no devices with significantly more “Not Sure”). There was a significant needle length effect on the probability of wheal formation. In particular, the chance of wheal formation decreases as the length of the needle increases. There was no significant “number of needle” effect.

TABLE 10
Summary of Wheal Formation

| | | Set Pressure | | |
|---|---|---|---|---|
| Needle Length | | 15 psi | 20 psi | Total |
| 1 | 1 mm | 18/18 = 100% | 20/20 = 100% | 38/38 = 100% |
| Needle | 1.5 mm | 16/19 = 84.2% | 17/19 = 89.5% | 33/38 = 86.8% |
| | 2 mm | 11/18 = 61.1% | 11/20 = 55% | 22/38 = 57.9% |
| | Total | 45/55 = 81.8% | 48/59 = 81.4% | 93/114 = 81.6% |
| 3 | 1 mm | 16/16 = 100% | NA | 552/750 = 73.6% |
| Needle | | | | |

6.2.6 Leakage

The number of times of incidence of leakage was observed for each device and where the leakage was observed for all injections (including the two failed injections) are summarized in Table 11 below. A chi-squared test of homogeneity indicated a significant difference in probability of leakage for the various devices tested. In particular, the 3 needle device has a higher probability of fluid than all other devices except 1 needle×1 mm, 20 psi. For volumes collected, the responses that were recorded as negative are converted to values of 0 μl.

TABLE 11
Fluid Observed Upon Removal of Device

| | | | Fluid Observed | | | | |
|---|---|---|---|---|---|---|---|
| | | | | | | | Average Volume Collected |
| Injection Type | | | | | | | (μl) |
| Treatment | Device | PSI | Yes | Device | Skin | Both | (Successful infusions only) |
| 1 | 1 needle × 1 mm | 15 | 5/20 | 3 | 1 | 1 | 0.11 |
| | | | (25%) | | | | |
| 2 | 1 needle × 1 mm | 20 | 8/20 | 5 | 1 | 2 | 0.16 |
| | | | (40%) | | | | |
| 3 | 1 needle × 1.5 mm | 15 | 5/20 | 2 | 3 | 0 | 0.07 |
| | | | (25%) | | | | |
| 4 | 1 needle × 1.5 mm | 20 | 2/20 | 1 | 1 | 0 | 0.30 |
| | | | (10%) | | | | |
| 5 | 1 needle × 2 mm | 15 | 2/20 | 0 | 2 | 0 | 0.02 |
| | | | (10%) | | | | |
| 6 | 1 needle × 2 mm | 20 | 2/20 | 1 | 1 | 0 | 0.07 |
| | | | (10%) | | | | |
| 7 | 3 needle × 1 mm | 15 | 12/20 | 9 | 1 | 2 | 0.48 |
| | | | (60%) | | | | |

Except for two instances (1 application failure for 1 needle×1.5 mm, 20 psi and 1 mechanical failure for the 3 needle device), the cause of the leakage was determined to be “weeping/pesky drop” in all cases.

6.2.7 Safety

Safety was assessed using the erythema and edema Draize scores, as described above. Table 12 shows the summary of the results.

TABLE 12
Draize Scores Summary

| Injection Type | | | Edema | | | Erythema | |
|---|---|---|---|---|---|---|---|
| Treatment | Device | PSI | 0 | 1 | 2 | 0 | 1 |
| 1 | 1 needle × 1 mm | 15 | 3 | 13 | 3 | 15 | 4 |
| 2 | 1 needle × 1 mm | 20 | 1 | 18 | 1 | 14 | 6 |
| 3 | 1 needle × 1.5 mm | 15 | 10 | 10 | 0 | 16 | 4 |
| 4 | 1 needle × 1.5 mm | 20 | 9 | 11 | 0 | 19 | 1 |
| 5 | 1 needle × 2 mm | 15 | 15 | 5 | 0 | 19 | 1 |
| 6 | 1 needle × 2 mm | 20 | 16 | 4 | 0 | 18 | 2 |
| 7 | 3 needle × 1 mm | 15 | 3 | 14 | 2 | 9 | 10 |

As shown in Table 12, there was a significant needle length effect for edema, with the 1 needle×1 mm device having a tendency for higher edema scores than the other 1 needle devices. There was also a significant “number of needle” effect for erythema, with the 3 needle device having a tendency for higher erythema scores than the 1 needle device.

6.2.2 Effects of Needle Spacing

To investigate the performance of linear array delivery and the effects of needle spacing, studies were performed using the following procedures: A total of 18 subjects received up to 9 infusions of preservative free sterile saline for injection in alternate thighs using each of the conditions described in Table 13 below with investigational microneedle protoytypes.

TABLE 13
Parameters for Needle Spacing Studies

| | | Rate | Volume | | Wait time |
|---|---|---|---|---|---|
| Treatment | Device | μL/min | (μL) | Site | (Min.) |
| A | L3 × 2 × 3 mm* | 100 μl/min | 250 | Thigh | 1* |
| B | L3 × 2 × 4.5 mm | 100 μl/min | 250 | Thigh | 1 |
| C | L3 × 2 × 3 mm | 250 μl/min | 250 | Thigh | 1 |
| D | L3 × 2 × 4.5 mm | 250 μl/min | 250 | Thigh | 1 |
| E | L3 × 2 × 3 mm | 500 μl/min | 250 | Thigh | 1 |
| F | L3 × 2 × 4.5 mm | 500 μl/min | 250 | Thigh | 1 |
| G | L3 × 2 × 3 mm | 500 μl/min | 500 | Thigh | 1 |
| H | L3 × 2 × 4.5 mm | 500 μl/min | 500 | Thigh | 1 |
| I | T3 × 2 × 4.5 mm | 100 μl/min | 250 | Thigh | 1 |

*L = linear array; T = triangular array (control); the number following L or T denotes number of needles; the number in the middle denotes needle length; and the final number denotes distance between each needles

The microneedle device was left on the skin for one minute following the infusion or injection (“wait time”). If increased leakage was noted due to excess weeping from device or injection site, the wait time was increased to 2 minutes. Injections were given in alternating thighs, starting at the upper, outer region then working in a Z pattern down the anterior thigh, alternating inner and outer thigh and right and left thigh. The infusion sequence was randomized for each subject.

The microneedle device consisted of three needles, 34-gauge by 1, 2 and 3 mm microneedles, housed in a Polycarbonate hub with an 18-inch section of polyvinylchloride/polyethylene with ethylvinylacetate tubing as a flow path. The device included an adhesive/foam ring used to secure the device to the subject's skin during infusion. The adhesive was double-coated 1/32″ white polyethylene foam with polyester liners. The adhesive ring was cut to fit around the perimeter of the device housing and applied to the device during assembly. Immediately before placing on the subjects, the release liner was removed by grasping the tabbed liner to expose the adhesive and the device placed on the subject's thigh, applying pressure to ensure contact of the adhesive with the subject's skin.

Devices were sterilized by ethylene oxide gas in accordance with ANSI/AAMI/ISO 11135-1994. EO Residual Information complies with ANSI/AAMI/ISO 10993-7.

A Sof-serter® Infusion Set Insertion System (MiniMed, Northridge Calif.) is a commercially available spring-loaded applicator manufactured to place an infusion set. This device has been modified to accept the Becton Dickinson Micromedica array catheter sets.

Pressure was monitored and recorded using Becton Dickinson DTX Plus TNF-R blood pressure transducer. The transducer was plumbed into the infusion system via a four-way stopcock. The transducer was connected using a single cable to a WPI TBM4M power supply/signal conditioner, which in turn passed on the amplified signal to a Fluke Hydra Data Bucket. The Data Bucket converts, digitizes, and caches the data until it is retrieved by a PC for storage and data processing. Alternatively, instead of the Fluke Hydra Data Bucket, a PC-based A/D data acquisition card was used to digitize the analog output from the WPI signal conditioner.

6.2.2.1 Statistical Analysis

For each injection (20*9=180 total), the completeness of injection was calculated as:

$$V = 1 - \frac{Leakage Volume}{Potential Injection Volume}$$

In this study, Potential Injection Volumes were 250 μL or 500 μL.

An ANOVA on V for treatments A, B, D, E, F & G (a 2×3 factorial sub-experiment) was performed using the following linear model:

Vᵢⱼₖₗₘ=RanGroupᵢ+Subject₍ᵢ₎ₗ(RanGroup)+Orderₖ+spacingₗ+rateₘ+spacing*rateₗₘ+noiseᵢⱼₗₖₘ

The following ANOVA table shows the degrees of freedom for each of the effects in this model:

| Effect | d.f. |
|---|---|
| Rangroup | 8 |
| sub(group) | 9 |
| order | 8 |
| spacing | 1 |
| rate | 2 |
| spacing * rate | 2 |
| noise | 77 |
| total | 107 |

RanGroup refers to the effect of randomization group, which corresponds to “orderings” of treatments; Subject is human experimental unit which was randomized to one of nine groups; Order refers to the order of treatment; spacing is one of 2 lengths (3.0 and 4.5 mm); rate is one of three possible infusion rates (100, 250 or 500 μl); spacing rate refers to the interaction effect between the two treatment variables; and noise is random fluctuations within any given experimental condition.

The root mean square error (root MSE) from this ANOVA was used to estimate the standard deviation of noise. For each treatment combination, the following metric was calculated:

$${K}_{l m} = \frac{{\overline{V}}_{l m} - Limit}{3 \sqrt{MSE}}$$

The subscripts l and m refer to each of the six combinations of needle lengths and body sites and V̅ₗₘ is the average value of V for treatment combination lm. The values of “Limit” were 0.90 and 0.95 (two calculations for each of the six treatment combinations). For each of the Kₗₘ, a 95% lower confidence limit was calculated using the formula described in Kotz, S., N. L. Johnson, Process capability Indices (Chapman Hall, London, 1993), p. 71, which is incorporated herein by reference:

$${K}_{l m} ( 95 ) = {K}_{l m} - {Z}_{0.95} \sqrt{\frac{1}{9 * ( d . f . e . + 1 )} + \frac{{K}_{l m}^{2}}{2 * ( d . f . e . )}}$$

The symbol “d.f.e.” represents the number of degrees of freedom for noise (error) based on the ANOVA.

The value of:

Φ(3Kₗₘ(95))

is therefore an approximate 95% lower confidence limit on the probability of either a 90% or 95% complete injection. The letter Φ represents the standard normal cumulative distribution function. Again, computations were made for both 90% and 95% complete injections. The assumptions underlying these computations are that the noise is normally distributed and that the variance is constant for all experimental conditions. If the assumptions of constant variance appear to be violated, a variance-stabilizing transformation may be employed. Based on 108 degrees of freedom for noise, the value of Kₗₘ must be at least 0.522 in order to be 95% confident that at least 90% of injections will be “complete.” The value of Kₗₘ must be at least 0.656 in order to be 95% confident that at least 95% of injections will be “complete.” The term “complete” means either 90% or 95% complete.

6.2.2.2 Leakage

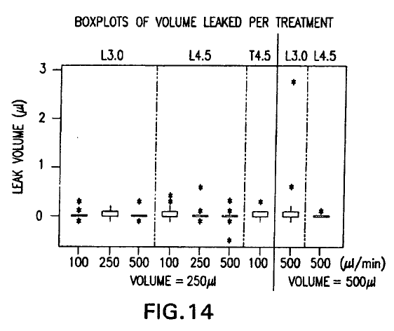

As shown in FIG. 14, with all devices succeeding in injecting more than 95% of the intended injection volume there was no significant treatment condition effect on the probability of leakage. Based on a sample size of 18, individual 95% lower bounds on the probability of successfully injecting 95% of 250 μl in the abdomen or deltoid with a 1 or 2 mm single-needle device was calculated to be 84.7%. In other words, there is a 95% confidence that the chance of injecting at least 95% of the intended volume for any of the treatments is at least 84.7%.

6.2.2.3 Back-Pressure

Table 14 below shows summary statistics of pressure measurements per treatment combination. The standard deviations in the table represent the total variability and contain a between donor component.

The definitions for the terms represented in Table 14 are as follows:

“maxp.0” refers to the maximum pressure from t0 to tf; “minp.1” refers to the minimum pressure from t1 to tf; “meanp.Hss” refers to the mean pressure from t2Hss to tfHss; “medianp.Hss” refers to the median pressure from t2Hss to tfHss; “minp.Hss” refers to the minimum pressure from t2Hss to tfHss; “maxp.Hss” refers to the maximum pressure from t2Hss to tfHss; “meanp.Lss” refers to the mean pressure from t2Lss to tfLss; “medianp.Lss” refers to the median pressure from t2Lss to tfLss; “minp.Lss” refers to the minimum pressure from t2Lss to tfLss; “maxp.Lss” refers to the maximum pressure from t2Lss to tfLss; “t0” refers to the time when the pressure has the first positive deviation from the baseline (beginning of injection); “t2” refers to the beginning time of the steady state (when there is one steady state, steady state being a stable pressure); “tf” refers to the end time when the device is shut off (end of injection); “t2Hss” refers to when there are 2 steady states this is the time when the “high” steady state begins, when there is 1 steady state this is t2; “tfHss” refers to when there are 2 steady states this is the time when the “high” steady state ends, when there is 1 steady state this is tf; “t2Lss” refers to when there are 2 steady states this is the time when the “low” steady state begins, when there is 1 steady state this is t2; “tfLess” refers to when there are 2 steady states this is the time when the “low” steady state ends, when there is 1 steady state this is tf.

TABLE 14
Summary Statistics for Back-Pressure

| | | Device | | | | | | | | |
|---|---|---|---|---|---|---|---|---|---|---|
| | | Volume = 250 μl | | | | | | | Volume = 500 μl | |
| | | | | | | | | T3 × 2 × | L3 × 2 × | L3 × 2 × |
| | | L3 × 2 × 3 mm | | | L3 × 2 × 4.5 mm | | | 4.5 mm | 3 mm | 4.5 mm |
| | | 100 μl/min | 250 μl/min | 500 μl/min | 100 μl/min | 250 μl/min | 500 μl/min | 100 μl/min | 500 μl/min | 500 μl/min |
| Maxp.0 | Mean | 2.327 | 3.800 | 6.507 | 2.124 | 3.355 | 5.760 | 2.155 | 6.411 | 5.974 |
| | Median | 2.174 | 2.998 | 6.335 | 1.712 | 3.187 | 5.058 | 2.034 | 6.068 | 5.616 |
| | SD | 0.982 | 2.036 | 2.627 | 1.080 | 1.088 | 1.886 | 0.888 | 1.737 | 2.039 |
| | Min | 0.731 | 1.951 | 3.209 | 0.642 | 1.94 | 3.701 | 0.764 | 3.287 | 3.399 |
| | Max | 4.62 | 9.053 | 15.415 | 4.294 | 5.678 | 9.272 | 4.037 | 10.071 | 10.595 |
| | n | 18 | 18 | 18 | 18 | 18 | 18 | 18 | 18 | 18 |
| Minp.1 | Mean | 1.095 | 2.015 | 3.652 | 0.998 | 1.854 | 4.029 | 1.052 | 3.600 | 3.870 |
| | Median | 0.997 | 1.968 | 3.802 | 0.697 | 1.634 | 3.684 | 1.019 | 3.427 | 3.612 |
| | SD | 0.736 | 0.879 | 1.143 | 0.651 | 0.846 | 1.232 | 0.441 | 1.351 | 1.032 |
| | Min | 0.177 | 0.686 | 1.684 | 0.199 | 0.653 | 2.897 | 0.409 | 1.041 | 2.418 |
| | Max | 3.366 | 4.317 | 5.848 | 2.63 | 4.485 | 7.722 | 1.984 | 6.311 | 5.961 |
| | n | 18 | 18 | 18 | 18 | 18 | 18 | 18 | 18 | 18 |
| Meanp.- | Mean | 1.683 | 3.985 | 4.630 | 1.193 | 2.385 | 4.830 | 1.635 | 5.411 | 5.371 |
| Hss | Median | 1.683 | 3.756 | 4.010 | 1.066 | 2.330 | 4.893 | 1.659 | 5.147 | 4.914 |
| | SD | * | 1.658 | 2.029 | 0.522 | 0.382 | 0.213 | 0.568 | 1.803 | 1.476 |
| | Min | 1.683 | 2.208 | 2.984 | 0.716 | 2.028 | 4.592 | 0.986 | 3.121 | 4.052 |
| | Max | 1.683 | 6.218 | 6.897 | 1.922 | 2.854 | 5.004 | 2.308 | 9.499 | 8.81 |
| | n | 1 | 4 | 3 | 4 | 4 | 3 | 5 | 10 | 8 |
| Medp.- | Mean | 1.673 | 3.906 | 4.638 | 1.191 | 2.382 | 4.807 | 1.636 | 5.400 | 5.358 |
| Hss | Median | 1.673 | 3.785 | 3.997 | 1.069 | 2.340 | 4.878 | 1.684 | 5.132 | 4.923 |
| | SD | * | 1.494 | 2.054 | 0.512 | 0.373 | 0.143 | 0.563 | 1.798 | 1.485 |
| | Min | 1.673 | 2.207 | 2.981 | 0.719 | 2.018 | 4.642 | 0.963 | 3.12 | 4.065 |
| | Max | 1.673 | 5.848 | 6.936 | 1.906 | 2.83 | 4.901 | 2.296 | 9.486 | 8.834 |
| | n | 1 | 4 | 3 | 4 | 4 | 3 | 5 | 10 | 8 |
| Minp.- | Mean | 1.529 | 3.365 | 4.380 | 1.022 | 2.229 | 4.436 | 1.414 | 4.941 | 5.105 |
| Hss | Median | 1.529 | 3.254 | 3.779 | 0.853 | 2.184 | 4.395 | 1.296 | 4.463 | 4.654 |
| | SD | * | 1.256 | 2.039 | 0.544 | 0.373 | 0.145 | 0.597 | 1.693 | 1.435 |
| | Min | 1.529 | 1.973 | 2.708 | 0.598 | 1.862 | 4.317 | 0.83 | 2.953 | 3.858 |
| | Max | 1.529 | 4.979 | 6.652 | 1.784 | 2.685 | 4.597 | 2.162 | 8.8 | 8.455 |
| | n | 1 | 4 | 3 | 4 | 4 | 3 | 5 | 10 | 8 |
| Maxp.- | Mean | 1.918 | 4.989 | 4.885 | 1.413 | 2.602 | 5.344 | 1.891 | 5.913 | 5.714 |
| Hss | Median | 1.918 | 4.205 | 4.362 | 1.346 | 2.513 | 5.238 | 2.006 | 5.605 | 5.256 |
| | SD | * | 2.785 | 1.989 | 0.527 | 0.429 | 0.593 | 0.594 | 1.954 | 1.499 |
| | Min | 1.918 | 2.552 | 3.209 | 0.841 | 2.24 | 4.811 | 1.185 | 3.287 | 4.25 |
| | Max | 1.918 | 8.995 | 7.083 | 2.118 | 3.142 | 5.983 | 2.541 | 0.071 | 9.145 |
| | n | 1 | 4 | 3 | 4 | 4 | 3 | 5 | 10 | 8 |
| Meanp.- | Mean | 1.363 | 2.395 | 4.579 | 1.236 | 2.176 | 4.395 | 1.231 | 4.318 | 4.239 |
| Lss | Median | 1.259 | 2.205 | 4.221 | 0.853 | 1.934 | 3.953 | 1.172 | 4.062 | 3.842 |
| | SD | 0.704 | 0.773 | 1.519 | 0.800 | 0.983 | 1.367 | 0.492 | 1.072 | 1.301 |
| | Min | 0.533 | 1.441 | 2.765 | 0.464 | 0.797 | 3.118 | 0.5 | 2.908 | 2.85 |
| | Max | 3.52 | 4.656 | 9.479 | 3.181 | 5.018 | 8.421 | 2.205 | 6.955 | 8.061 |
| | n | 18 | 18 | 18 | 18 | 18 | 18 | 18 | 18 | 18 |
| Medp.- | Mean | 1.355 | 2.382 | 4.555 | 1.224 | 2.166 | 4.383 | 1.218 | 4.298 | 4.232 |
| Lss | Median | 1.257 | 2.195 | 4.211 | 0.841 | 1.929 | 3.936 | 1.163 | 4.040 | 3.830 |
| | SD | 0.704 | 0.770 | 1.494 | 0.788 | 0.984 | 1.372 | 0.491 | 1.077 | 1.347 |
| | Min | 0.531 | 1.44 | 2.774 | 0.454 | 0.786 | 3.098 | 0.487 | 2.886 | 2.808 |
| | Max | 3.5 | 4.642 | 9.342 | 3.109 | 4.968 | 8.432 | 2.195 | 6.947 | 8.277 |
| | n | 18 | 18 | 18 | 18 | 18 | 18 | 18 | 18 | 18 |
| Minp.- | Mean | 1.178 | 2.209 | 4.181 | 1.040 | 1.906 | 4.036 | 1.059 | 3.993 | 3.889 |
| Lss | Median | 1.080 | 1.995 | 3.897 | 0.731 | 1.707 | 3.684 | 1.019 | 3.768 | 3.612 |
| | SD | 0.671 | 0.728 | 1.350 | 0.673 | 0.839 | 1.235 | 0.447 | 0.997 | 1.023 |
| | Min | 0.432 | 1.329 | 2.618 | 0.376 | 0.653 | 2.897 | 0.409 | 2.663 | 2.641 |
| | Max | 3.366 | 4.317 | 8.57 | 2.63 | 4.485 | 7.722 | 2.029 | 6.221 | 5.961 |
| | n | 18 | 18 | 18 | 18 | 18 | 18 | 18 | 18 | 18 |
| Maxp.- | Mean | 1.591 | 2.678 | 5.151 | 1.509 | 2.534 | 4.846 | 1.502 | 4.767 | 4.682 |
| Lss | Median | 1.424 | 2.602 | 4.637 | 1.108 | 2.357 | 4.273 | 1.374 | 4.525 | 4.244 |
| | SD | 0.750 | 0.827 | 1.881 | 0.998 | 1.159 | 1.516 | 0.546 | 1.166 | 1.508 |
| | Min | 0.653 | 1.595 | 2.964 | 0.575 | 1.008 | 3.433 | 0.664 | 3.198 | 3.198 |
| | Max | 3.723 | 5.024 | 11.226 | 4.294 | 5.678 | 9.099 | 2.44 | 7.848 | 9.561 |
| | n | 18 | 18 | 18 | 18 | 18 | 18 | 18 | 18 | 18 |

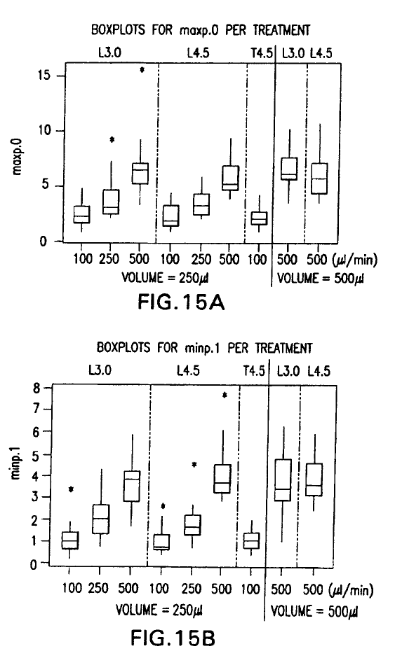

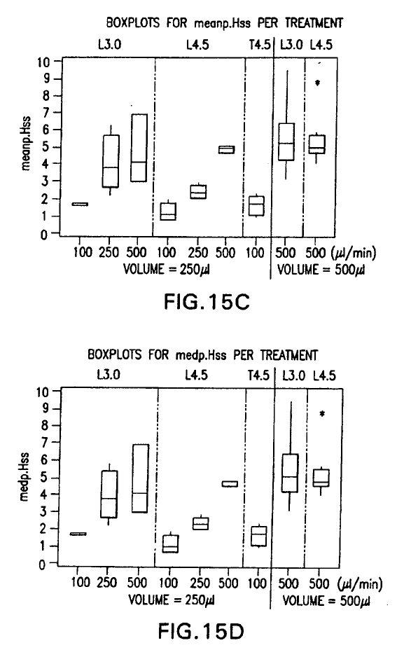

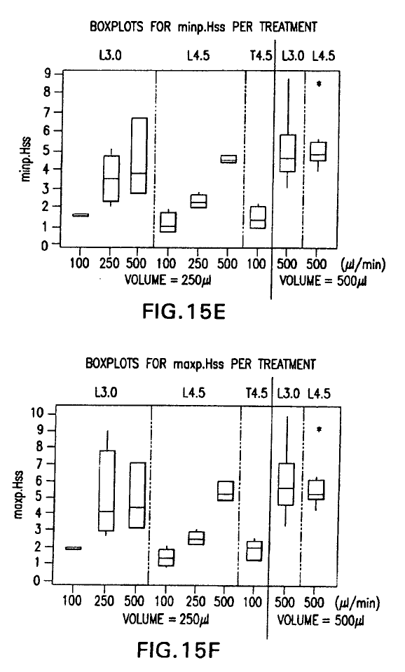

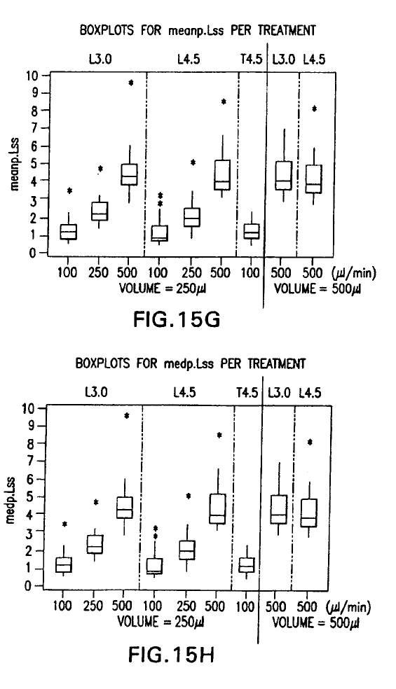

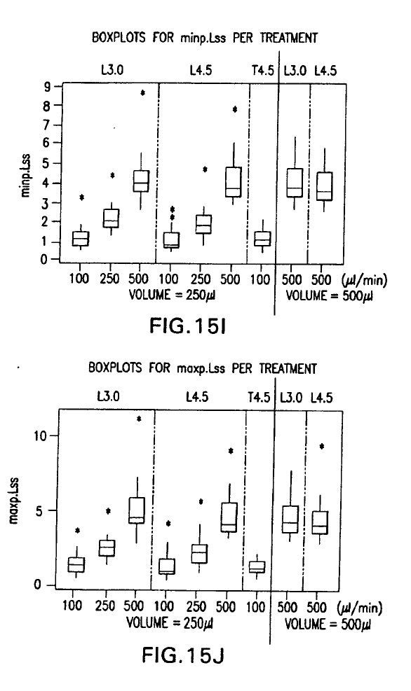

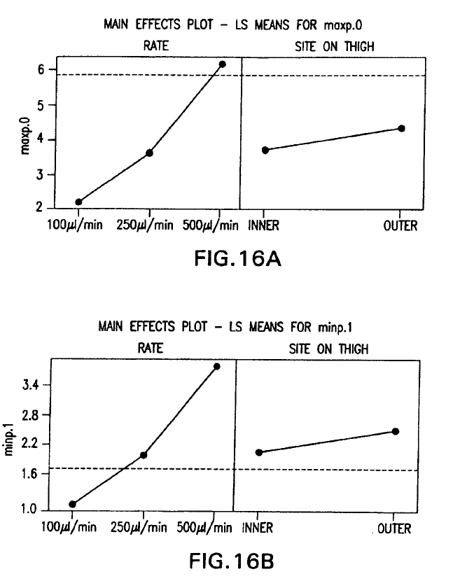

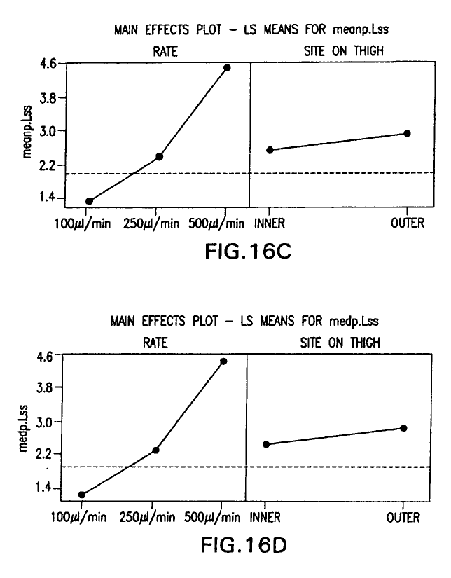

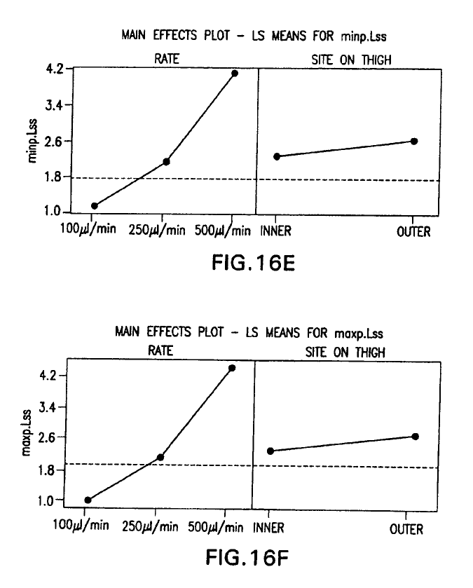

The distribution of pressure measurements are shown in FIGS. 15-17.

6.2.2.4 Treatment Effects on Pressure Measurement

For evaluation treatments A-F and E-H, maxp.0, minp.0, meanp.Hss, medp.Hss, minp.Hss, maxp.Hss, meanp.Lss, medp.Lss, minp.Lss & maxp.Lss values were analyzed using ANOVA. The first ANOVA model included subject-to-subject differences, order of injection, site (inner or outer), spacing and rate main effects and spacing by rate interactions. The second ANOVA model included subject-to-subject differences, order of injection, site (inner or outer), spacing and volume main effects and spacing by volume interactions. Treatments B & I were also compared to determine whether a significant difference exists between linear and triangular arrays (with spacing of 4.5 mm, rate of 100 μl/min and volume of 250 μl). Results were as following:

### Maxp.0 & Minn.0:

Treatments A-F: The subject, site on thigh and rate effects were significant.
Treatments E-H: The subject and site on thigh effects were significant.
Treatments B & I: No significant effects.
   Meanp.Hss, medp.Hss, minp.Hss, maxp.Hss
Treatments C-F: No significant effects.
Treatments E-H: No significant effects.
Treatments B & I: No significant effects.
   Meanp.Lss, medp.Lss, minp.Lss. maxp.Lss:
Treatments A-F: The subject, site on thigh and rate effects were significant.
Treatments E-H: The subject and site on thigh effects were significant.
Treatments B & I: No significant effects.

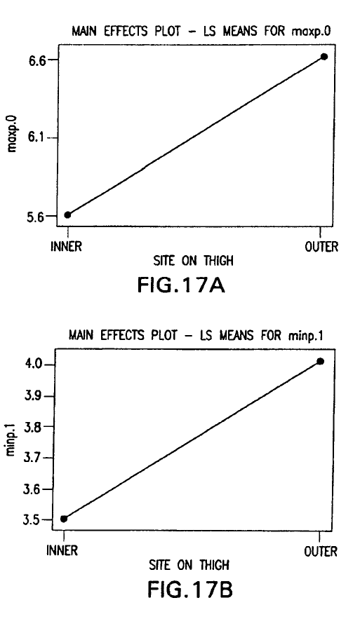

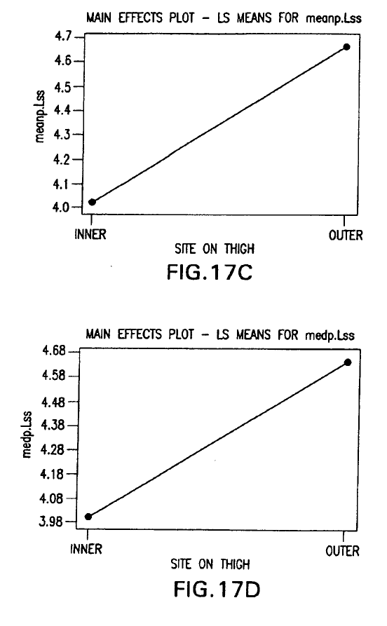

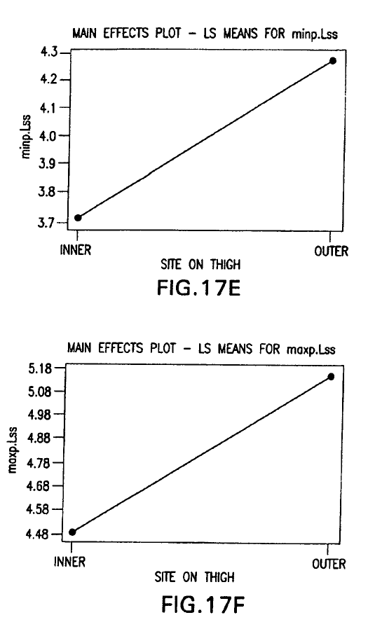

The size and magnitude of the significant main effects are shown in FIG. 17. Table 15 presents the significant differences from the multiple comparisons for the rate and site on thigh (treatment A-F). Results for rates and site on thigh are averaged over the two devices. For every difference represented in the table, if the confidence interval does not contain the value 0, it indicates a statistically significant difference.

TABLE 15
Average and 95% Confidence Intervals for the Differences
in Factor Levels (3.0 &4.5 mm needle spacing)

| | | | | Site on Thigh |
|---|---|---|---|---|
| | Delivery Rate Biases or Differences | | | Biases or |
| | 250 μl/min- | 500 μl/min- | 500 μl/min- | Differences |
| Response | 100 μl/min | 100 μl/min | 250 μl/min | Outer-Inner |
| Maxp.0 | 1.35 | 3.91 | 2.56 | 0.61 |
| | (0.49, 2.21) | (3.05, 4.77) | (1.70, 3.42) | (0.01, 1.21) |
| Minp.0 | 0.89 | 2.79 | 1.91 | 0.42 |
| | (0.43, 1.34) | (2.34, 3.25) | (1.45, 2.36) | (0.10, 0.74) |
| Meanp.Lss | 0.99 | 3.19 | 2.20 | 0.38 |
| | (0.48, 1.49) | (2.68, 3.69) | (1.70, 2.71) | (0.03, 0.74) |
| Medp.Lss | 0.98 | 3.18 | 2.20 | 0.39 |
| | (0.48, 1.49) | (2.68, 3.68) | (1.69, 2.70) | (0.03, 0.74) |
| Minp.Lss | 0.95 | 3.00 | 2.05 | 0.34 |
| | (0.49, 1.41) | (2.54, 3.46) | (1.59, 2.51) | (0.02, 0.66) |
| Maxp.Lss | 1.06 | 3.45 | 2.39 | 0.41 |
| | (0.47, 1.64) | (2.86, 4.04) | (1.80, 2.98) | (−0.01, 0.82) |

6.2.2.5 Pain

Summary statistics of the needlestick and end of injection pain and unpleasantness per treatment combination are presented in Tables 16 and 17 below.

TABLE 16
Pain Intensity

| | | Device | | | | | | | | |
|---|---|---|---|---|---|---|---|---|---|---|
| | | Volume = 250 μl | | | | | | | Volume = 500 μl | |
| | | | | | | | | T3 × 2 × | L3 × 2 × | L3 × 2 × |
| | | L3 × 2 × 3 mm | | | L3 × 2 × 4.5 mm | | | 4.5 mm | 3 mm | 4.5 mm |
| | | 100 μl/min | 250 μl/min | 500 μl/min | 100 μl/min | 250 μl/min | 500 μl/min | 100 μl/min | 500 μl/min | 500 μl/min |
| Needle | Mean | 1.4 | 1.3 | 0.8 | 1.9 | 0.7 | 1.1 | 1.0 | 1.5 | 1.7 |
| Stick | Median | 1.0 | 1.0 | 0.0 | 0.5 | 0.0 | 0.0 | 0.0 | 1.0 | 0.0 |
| | SD | 2.3 | 1.5 | 1.2 | 2.6 | 0.9 | 1.4 | 1.4 | 2.8 | 3.4 |
| | Min | 0 | 0 | 0 | 0 | 0 | 0 | 0 | 0 | 0 |
| | Max | 8 | 5 | 4 | 8 | 3 | 4 | 4 | 12 | 13 |
| | n | 18 | 18 | 18 | 18 | 18 | 18 | 18 | 18 | 18 |
| After | Mean | 1.7 | 1.5 | 2.9 | 2.1 | 2.0 | 2.7 | 1.8 | 3.2 | 3.8 |
| entire | Median | 1.0 | 1.0 | 2.0 | 2.0 | 1.5 | 2.0 | 1.0 | 2.0 | 3.5 |
| dose | SD | 1.7 | 1.6 | 2.9 | 2.0 | 1.8 | 2.6 | 2.9 | 3.2 | 3.5 |
| | Min | 0 | 0 | 0 | 0 | 0 | 0 | 0 | 0 | 0 |
| | Max | 6 | 5 | 9 | 7 | 6 | 9 | 10 | 11 | 11 |
| | n | 18 | 18 | 18 | 18 | 18 | 18 | 18 | 18 | 18 |

TABLE 17
Unpleasantness

| | | Device | | | | | | | | |
|---|---|---|---|---|---|---|---|---|---|---|
| | | Volume = 250 μl | | | | | | | Volume = 500 μl | |
| | | | | | | | | T3 × 2 × | L3 × 2 × | L3 × 2 × |
| | | L3 × 2 × 3 mm | | | L3 × 2 × 4.5 mm | | | 4.5 mm | 3 mm | 4.5 mm |
| | | 100 μl/min | 250 μl/min | 500 μl/min | 100 μl/min | 250 μl/min | 500 μl/min | 100 μl/min | 500 μl/min | 500 μl/min |
| Needle | Mean | 1.0 | 1.2 | 0.7 | 1.5 | 0.6 | 1.3 | 0.6 | 1.3 | 1.3 |
| Stick | Median | 2.0 | 1.8 | 1.4 | 2.2 | 1.0 | 2.0 | 0.8 | 2.7 | 3.0 |
| | SD | 0.0 | 0.5 | 0.0 | 0.0 | 0.0 | 0.0 | 0.0 | 0.0 | 0.0 |
| | Min | 0 | 0 | 0 | 0 | 0 | 0 | 0 | 0 | 0 |
| | Max | 7 | 6 | 5 | 7 | 4 | 6 | 2 | 11 | 12 |
| | n | 18 | 18 | 18 | 18 | 18 | 18 | 18 | 18 | 18 |
| After | Mean | 1.6 | 1.2 | 3.0 | 2.1 | 1.9 | 2.2 | 1.9 | 2.9 | 3.0 |
| entire | Median | 1.7 | 1.7 | 2.4 | 2.2 | 2.0 | 2.1 | 2.3 | 2.6 | 2.8 |
| dose | SD | 1.0 | 0.5 | 3.0 | 1.5 | 1.0 | 1.0 | 1.0 | 2.5 | 2.5 |
| | Min | 0 | 0 | 0 | 0 | 0 | 0 | 0 | 0 | 0 |
| | Max | 5 | 7 | 7 | 7 | 5 | 7 | 8 | 8 | 8 |
| | n | 18 | 18 | 18 | 18 | 18 | 18 | 18 | 18 | 18 |

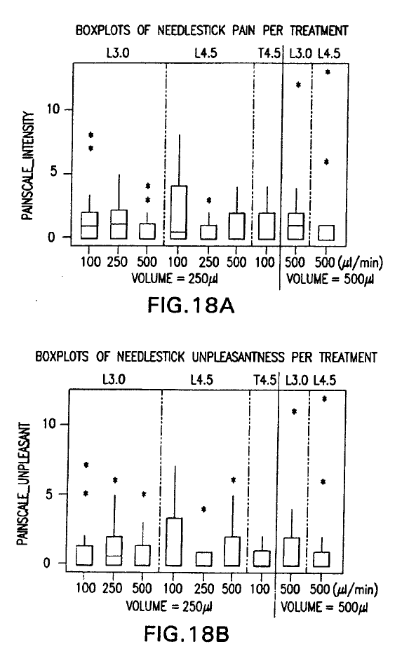

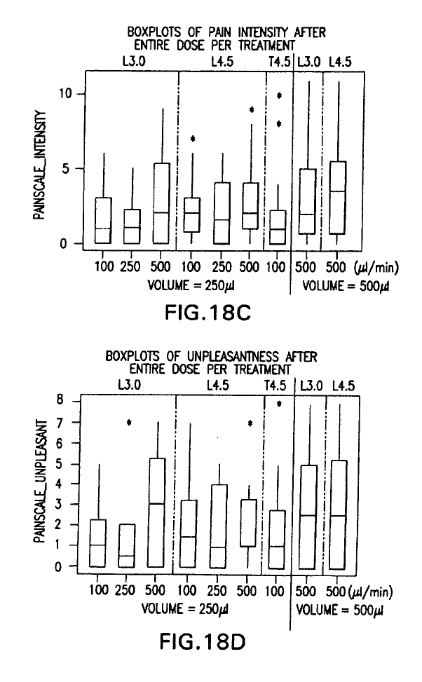

The distribution of pain and unpleasantness scores are shown in FIG. 18.

Paired t-tests were performed with the data at needlestick and at end of entire dose to determine if any difference existed between the two scales. For data obtained at needlestick, statistically significant difference between the two scales was observed, with the pain intensity scale resulting in an average score 0.2 pain units higher than the unpleasantness scale (95% confidence interval of (0.05, 0.35)). For those obtained at the end of entire dose, no statistically significant difference was observed.

6.2.2.6 Treatment Effects on Pain and Unpleasantness

For evaluation of treatments A-F and E-H, pain intensity and pain unpleasantness values (needle stick and after entire dose) were analyzed using ANOVA. The first ANOVA model included subject-to-subject differences, time recorded (needle stick or after entire dose), order of injection, site (inner or outer), spacing and rate main effects and spacing by rate, spacing by time recorded and rate by time recorded interactions. The second ANOVA model included subject-to-subject differences, time recorded (needle stick or after entire dose), order of injection, site (inner or outer), spacing and volume main effects and spacing by volume, spacing by time recorded and rate by time recorded interactions. Treatments B & I were also compared to determine whether a significant difference exists between linear and triangular arrays (with spacing of 4.5 mm, rate of 100 μl/min and volume of 250 μl). The following results were observed:

### Painscale Intensity:

Treatments A-F: The subject and time recorded effects were significant and the time recorded by rate interaction was significant. The average pain intensity after the entire dose was significantly higher for the rate of 500 μl/min than for the rate of 250 μl/min (average difference of 1 pain unit).
Treatments E-H: The only significant effects were subject and time recorded.
Treatments B & I: The only significant effect was subject.

### Painscale Unpleasantness:

Treatments A-F: The only significant effects were subject and time recorded
Treatments E-H: The only significant effects were subject and time recorded.
Treatments B & I: The only significant effects were subject and time recorded.

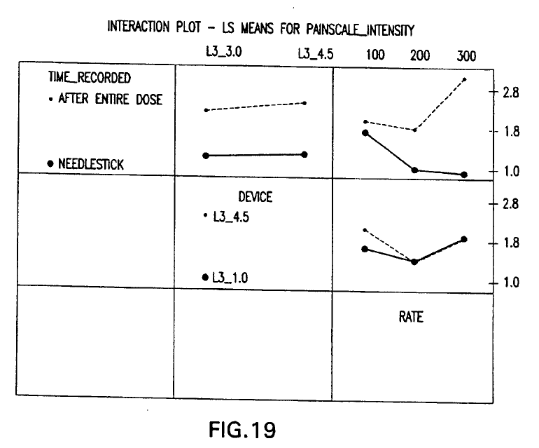

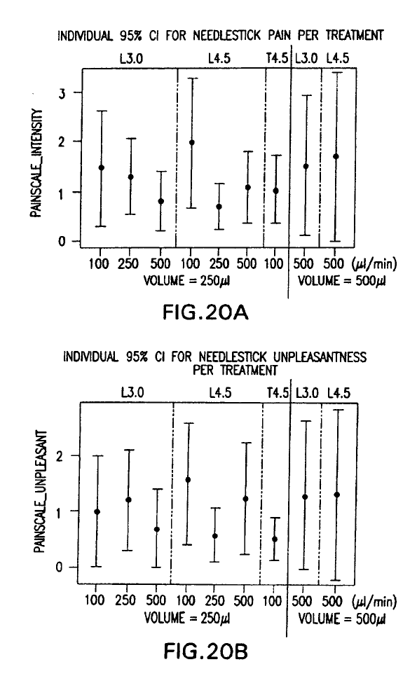

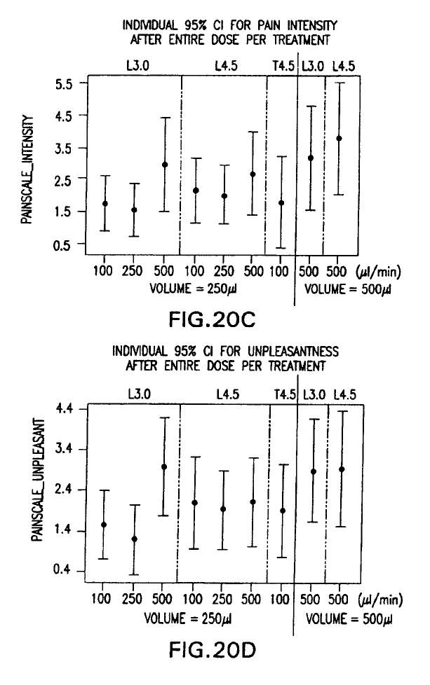

The size and magnitude of the significant rate by time recorded interaction for painscale intensity are shown in FIG. 19. In addition, individual confidence intervals for average pain per device in original units are shown in FIG. 20.

6.2.2.7 Wheal

Table 18 summarizes the number and percent wheals for each device. There are no significant differences between any of the devices.

TABLE 18
Summary of Wheal Formation

| | | | | 95% |
|---|---|---|---|---|
| | | | | lower bound |
| | | | | on probability |
| Volume | Device | Rate | Wheal | of wheal |
| 250 μl | L3 × 2 × 3 mm | 100 μl/min | 6/18 = 33.3% | 15.6% |
| | | 250 μl/min | 4/18 = 22.2% | 8.0% |
| | | 500 μl/min | 5/18 = 27.8% | 11.6% |
| | L3 × 2 × 4.5 mm | 100 μl/min | 4/18 = 22.2% | 8.0% |
| | | 250 μl/min | 5/18 = 27.8% | 11.6% |
| | | 500 μl/min | 6/18 = 33.3% | 15.6% |
| | T3 × 2 × 4.5 mm | 100 μl/min | 8/18 = 44.4% | 24.4% |
| 500 μl | L3 × 2 × 3 mm | 500 μl/min | 8/18 = 44.4% | 24.4% |
| | L3 × 2 × 4.5 mm | 500 μl/min | 6/18 = 33.3% | 15.6% |

Leakage

Table 19 summarizes the number of times leakage was observed for each device and where the leakage was observed for all injections. There is no significant difference between the treatments.

TABLE 19
Leakage

| | | | Fluid Seen | | | |
|---|---|---|---|---|---|---|
| Volume | Device | Rate | Yes | Device | Skin | Both |
| 250 μl | L3 × 2 × | 100 μl/min | 0/18 = 0% | 0 | 0 | 0 |
| | 3 mm | 250 μl/min | 0/18 = 0% | 0 | 0 | 0 |
| | | 500 μl/min | 2/18 = 11.1% | 2 | 0 | 0 |
| | L3 × 2 × | 100 μl/min | 2/18 = 11.1% | 1 | 1 | 0 |
| | 4.5 mm | 250 μl/min | 0/18 = 0% | 0 | 0 | 0 |
| | | 500 μl/min | 0/18 = 0% | 0 | 0 | 0 |
| | T3 × 2 × | 100 μl/min | 0/18 = 0% | 0 | 0 | 0 |
| | 4.5 mm | | | | | |
| 500 μl | L3 × 2 × | 500 μl/min | 0/18 = 0% | 0 | 0 | 0 |
| | 3 mm | | | | | |
| | L3 × 2 × | 500 μl/min | 1/18 = 5.6% | 1 | 0 | 0 |
| | 4.5 mm | | | | | |

The cause of leakage was marked as “weeping/pesky drop” in all incidents.

6.2.2.9 Safety

Safety was assessed using the erythema and edema Draize scores, as described above. Tables 20 and 21 show the summary of the results.

TABLE 20
Erythema Summary

| | | | Erythema Scores | | | | | | | | | | | |
|---|---|---|---|---|---|---|---|---|---|---|---|---|---|---|
| | | | Time | | | | | | | | | | | |
| | | | 0 hr | | 1 hr | | 2 hr | | 3 hr | | 6 hr | | 24 hr | |
| Volume | Device | Rate | 0 | 1 | 0 | 1 | 0 | 1 | 0 | 1 | 0 | 1 | 0 | 1 |
| 250 μl | L3 × 2 × | 100 μl/min | 10 | 8 | 16 | 2 | 18 | 0 | 18 | 0 | 18 | 0 | 18 | 0 |
| | 3 mm | 250 μl/min | 13 | 5 | 16 | 2 | 17 | 1 | 17 | 1 | 18 | 0 | 18 | 0 |
| | | 500 μl/min | 9 | 9 | 15 | 3 | 15 | 3 | 16 | 2 | 17 | 1 | 17 | 1 |
| | L3 × 2 × | 100 μl/min | 11 | 7 | 17 | 1 | 17 | 1 | 17 | 1 | 18 | 0 | 18 | 0 |
| | 4.5 mm | 250 μl/min | 12 | 6 | 17 | 1 | 18 | 0 | 18 | 0 | 18 | 0 | 18 | 0 |
| | | 500 μl/min | 13 | 5 | 15 | 3 | 17 | 1 | 17 | 1 | 16 | 2 | 17 | 1 |
| | T3 × 2 × | 100 μl/min | 9 | 9 | 14 | 4 | 16 | 2 | 18 | 0 | 18 | 0 | 18 | 0 |
| | 4.5 mm | | | | | | | | | | | | | |
| 500 μl | L3 × 2 × | 500 μl/min | 12 | 6 | 17 | 1 | 17 | 1 | 17 | 1 | 17 | 1 | 18 | 0 |
| | 3 mm | | | | | | | | | | | | | |
| | L3 × 2 × | 500 μl/min | 11 | 7 | 15 | 3 | 16 | 2 | 17 | 1 | 17 | 1 | 18 | 0 |
| | 4.5 mm | | | | | | | | | | | | | |

TABLE 21
Edema Summary

| | | | Edema Scores | | | | | | | | | | | |
|---|---|---|---|---|---|---|---|---|---|---|---|---|---|---|
| | | | Time | | | | | | | | | | | |
| | | | 0 hr | | 1 hr | | 2 hr | | 3 hr | | 6 hr | | 24 hr | |
| Volume | Device | Rate | 0 | 1 | 0 | 1 | 0 | 1 | 0 | 1 | 0 | 1 | 0 | 1 |
| 250 μl | L3 × 2 × | 100 μl/min | 14 | 4 | 15 | 3 | 16 | 2 | 18 | 0 | 18 | 0 | 18 | 0 |
| | 3 mm | 250 μl/min | 10 | 8 | 17 | 1 | 18 | 0 | 18 | 0 | 18 | 0 | 18 | 0 |
| | | 500 μl/min | 13 | 5 | 15 | 3 | 16 | 2 | 18 | 0 | 18 | 0 | 18 | 0 |
| | L3 × 2 × | 100 μl/min | 13 | 5 | 18 | 0 | 18 | 0 | 18 | 0 | 18 | 0 | 18 | 0 |
| | 4.5 mm | 250 μl/min | 17 | 1 | 17 | 1 | 18 | 0 | 18 | 0 | 18 | 0 | 18 | 0 |
| | | 500 μl/min | 15 | 3 | 16 | 2 | 18 | 0 | 18 | 0 | 18 | 0 | 18 | 0 |
| | T3 × 2 × | 100 μl/min | 14 | 4 | 16 | 2 | 17 | 1 | 17 | 1 | 18 | 0 | 18 | 0 |
| | 4.5 mm | | | | | | | | | | | | | |
| 500 μl | L3 × 2 × | 500 μl/min | 15 | 3 | 16 | 2 | 18 | 0 | 18 | 0 | 18 | 0 | 17 | 1 |
| | 3 mm | | | | | | | | | | | | | |
| | L3 × 2 × | 500 μl/min | 14 | 4 | 16 | 2 | 17 | 1 | 18 | 0 | 18 | 0 | 18 | 0 |
| | 4.5 mm | | | | | | | | | | | | | |

6.2.2.10 Overall Preference

Table 22 summarizes the number of times each device was chosen as least painful and most painful. There is no significant difference between the devices.

TABLE 22
Overall Preference

| Volume | Device | Rate | Least Painful | Most Painful |
|---|---|---|---|---|
| 250 μl | L3 × 2 × 3 mm | 100 μl/min | 2 | 1 |
| | | 250 μl/min | 2 | 1 |
| | | 500 μl/min | 2 | 3 |
| | L3 × 2 × 4.5 mm | 100 μl/min | 1 | 1 |
| | | 250 μl/min | 1 | 2 |
| | | 500 μl/min | 1 | 1 |
| | T3 × 2 × 4.5 mm | 100 μl/min | 3 | 1 |
| 500 μl | L3 × 2 × 3 mm | 500 μl/min | 0 | 1 |
| | L3 × 2 × 4.5 mm | 500 μl/min | 0 | 5 |

6.3 Constant Pressure Infusion Using N2 Mediated Infusion

6.3.1 Constant Pressure Infusion

A series of studies were performed using constant pressure infusion system, with varied parameters as indicted in the following sections. Although each study was performed according to specific sets of parameters, the protocols of all studies can be broadly summarized as the following.

6.3.2 Pressure Control System

Infusion pressure was controlled by a nitrogen gas pressure control system. An ultra-high purity cylinder (National/Specialty Gases UHP grade size 80), equipped with a high purity single stage regulator (Matheson Model# 3539-580), was used. Nitrogen pressure was stepped down from cylinder pressure to 50 psi, passed through a transfer line to a second precision regulator (Ingersoll-Rand PR4021-300). This regulator was used to reduce the pressure to the level used for infusion. Nitrogen was then passed through a tee connector equipped with a digital readout pressure gauge (NeTech part#200-2000PS). The digital gauge indicates the pressure of infusion. Downstream from the gauge was a three-way stopcock used to admit to headspace of a saline reservoir (factory sealed 10 mL glass saline vial) or vent off pressure during vial replacement. The exit port of the stopcock was fitted with a filter (Millipore 25 mm 0.22 μm, part # SLGVS-25US) to ensure cleanliness and sterility of the nitrogen gas admitted to the headspace of the saline vial.

6.3.3 Flow Monitoring

Flow rate measurement was accomplished by continuous gravimetric monitoring of the saline reservoir throughout the entire delivery process. The saline reservoir was placed on an analytical balance, which automatically records changes in mass over time to a computerized data file. Mass changes can be converted to flow over time by adjusting for the density of the delivery fluid, saline. Flow initiation and cessation were manually controlled via the stopcock in the upstream fluid path between the saline reservoir and the microneedle catheter set.

6.3.4 Data Collection

6.3.4.1 Performance (Efficacy)

Efficacy were determined by completeness of infusion, i.e., saline not delivered or saline which leaks out of the infusion site. A complete or successful infusion/injection is defined as less than or equal to 10% leakage of total fluid volume delivered as determined by gravimetric methodology.

Gravimetric Methodology-Leakage out of infusion site or failure of fluid to enter skin, were assessed immediately following each infusion as follows: After removal of the device, a pre-weighed absorbent swab was placed against the skin or the device to collect any visible fluid that leaked out or did not penetrate the skin. The swab was re-weighed and the fluid volume calculated.

6.3.4.2 Pressure

Pressure was monitored and recorded using a Becton Dickinson DTX Plus TNF-R blood pressure transducer approved for human use. The transducer was plumbed into the infusion system via a four-way stopcock. The transducer was connected using a single cable to a WPI TBM4M power supply/signal conditioner, which in turn passes on the amplified signal to a Fluke Hydra Data Bucket. The Data Bucket converts, digitizes, and caches the data until it is retrieved by a PC for storage and data processing. Alternatively, instead of the Fluke Hydra Data Bucket, a PC-based A/D data acquisition card was used to digitize the analog output from the WPI signal conditioner.

6.3.4.3. Safety

Safety was determined by assessing the development of any adverse skin effects at various times following infusion using the Draize scoring method.

Draize Scoring Scale

| | |
|---|---|
| Erythema | Edema |

| | | | |
|---|---|---|---|
| No erythema | 0 | No edema | 0 |
| Very slight erythema | 1 | Very slight edema | 1 |
| Well defined erythema | 2 | Slight edema | 2 |
| Moderate to severe erythema | 3 | Moderate edema | 3 |
| Severe Erythema (beet-red to | 4 | Severe edema | 4 |
| eshar formation) | | | |

Draize scoring and assessment of any other cutaneous events were done by the Study Staff immediately following all treatments. At that time, the subject was instructed how to perform Draize scoring and asked to continue to make observations at 1, 2, 3, 6 and 24 hrs+/−30 minutes post treatment.

6.3.5 Pain

Pain was determined using a Gracely Box SL Scale for Pain Intensity (scale of from 0 (no pain sensation) to 20 (extremely intense for 18 and up)) and the Gracely Pain Unpleasantness scale (scale of from 0 (neutral) to 20 (very intolerable for 17 and up)). treatments, pain and unpleasantness perceived by the subject was evaluated twice during each treatment. First, after the device has been applied, the subject was asked to rate the pain perceived at that moment following the needle stick. Second, after the total dose has been infused the subject was asked to rate the overall perceived pain for the entire infusion process, including needle stick.

6.3.6 Wheal Formation

After infusion device was removed from the skin, information about the wheal (e.g., presence of a wheal) was observed and recorded.

6.3.7 Preference

Following the completion of all injections, subject was asked to respond to the following questions:

1. Was there one injection that stood out as being the “least painful”?—If answer is YES, please indicate which site (1-9).

2. Was there one injection that stood out as being the “most painful”?—If answer is YES, please indicate which site (1-9).

6.3.8 Statistical Analysis

In all cases, p-values less than 0.05 was considered significant.

6.3.8.1 Fluid Flow Rate

Fluid flow rate (peak and average) was analyzed by ANOVA using the following linear model:

Yᵢⱼₖₗₘ=RanGroupᵢ+Subject₍ᵢ₎ⱼ(RanGroup)+Orderₖ+deviceₗ+pressureₘ+siteₙ+deviceₗ*pressureₘ+deviceₗ*siteₙ+pressureₘ*siteₙ+noiseᵢⱼₖₗₘ

Post-hoc multiple comparisons were performed for significant main factor and interaction effects. The post-hoc comparisons helped identify which levels or combination of levels actually differ from each other and by how much on average (with 95% confidence interval).

6.3.8.2 Major Leakage

Because minimal leakage with non-normally distributed volumes was expected, an analysis of the actual leakage volume was not possible. Individual 95% upper bounds on the probability of major leakage (failure) was obtained for each treatment. If no failures were observed with sample sizes of 24, 95% individual upper bounds on the probability of failure of 11.7% would be obtained.

6.3.8.3 Fluid Delivery Duration and Pain of Infusion

Fluid delivery duration and pain of infusion were analyzed using the method for determining the fluid flow rate described above. Post-hoc multiple comparisons were performed if the factor effects or interaction were significant. The post-hoc comparisons helped identify which levels or combination of levels actually differ from each other and by how much on average (with 95% confidence interval).

Responses using 0-3 or 0-4 scales (Draize scores, bleeding) were summarized per factor level combinations and compared via Chi-Squared tests of homogeneity or ordinal logistic regression. Binary responses were summarized per factor level combinations and compared using Fisher's exact test or binary logistic regression.

6.3.9 Experimental Design

To investigate the effects of device type and pressure on various characteristics of fluid delivery, studies were performed using the following procedures. A total of 20 subjects received up to 10 injections of sterile non-bacteriostatic saline for injection in alternate thighs using each of the conditions described in Table 23 below.

TABLE 23
Parameters for N2 Mediated Infusion

| Treatment | Device | PSI | Volume | Site | Wait time |
|---|---|---|---|---|---|
| A | 1 × 2 mm | 10 | 500 | Thigh | At least 1 min. |
| B | L3 × 2 mm × 3 mm | 10 | 500 | Thigh | At least 1 min. |
| | needle spacing | | | | |
| C | 1 × 2 mm | 15 | 500 | Thigh | At least 1 min. |
| D | L3 × 2 mm × 3 mm | 15 | 500 | Thigh | At least 1 min. |
| | needle spacing | | | | |
| E | 1 × 2 mm | 17.5 | 500 | Thigh | At least 1 min. |
| F | L3 × 2 mm × 3 mm | 17.5 | 500 | Thigh | At least 1 min. |
| | needle spacing | | | | |
| G | 1 × 2 mm | 20 | 500 | Thigh | At least 1 min. |
| H | L3 × 2 mm × 3 mm | 20 | 500 | Thigh | At least 1 min. |
| | needle spacing | | | | |
| I | 1 × 2 mm | 25 | 500 | Thigh | At least 1 min. |
| J | L3 × 2 mm × 3 mm | 25 | 500 | Thigh | At least 1 min. |
| | needle spacing | | | | |

One and three needle infusions at the same pressure were administered consecutively at adjacent sites (i.e., after approximately 1 minute “rest” and within 3 cm of each other). The order of administration of the injections at various pressures was randomized prior to the study.

The two infusions of the same pressure were delivered adjacent to one another on opposite sides of the midline of the anterior thigh. The next pair of infusions were delivered to the contra-lateral thigh. The randomization scheme was determined prior to the study, but assignment was not made until after a subject was enrolled.

6.3.9.1 Flow Rate

Table 24 below shows summary statistics of flow rate measurements per treatment combination. The standard deviations in the table represent the total variability and contain a between donor component.

TABLE 24
Summary Statistics

| | | Device | | | | | | | | | |
|---|---|---|---|---|---|---|---|---|---|---|---|
| | | 1 × 2 mm | | | | | L3 × 2 mm × 3 | | | | |
| | | 10 PSI | 15 PSI | 17.5 PSI | 20 PSI | 25 PSI | 10 PSI | 15 PSI | 17.5 PSI | 20 PSI | 25 PSI |
| Flow | Mean | 497.66 | 771.15 | 810.43 | 960.21 | 1134.59 | 1177.28 | 1587.42 | 2000.50 | 2345.60 | 2910.26 |
| Rate | Median | 540.05 | 744.1 | 917.54 | 1040.89 | 1272.66 | 1227.79 | 1722.01 | 2075.91 | 2567.52 | 3174.66 |
| (all) | Std. | 116.09 | 353.06 | 241.71 | 224.56 | 357.66 | 238.38 | 506.76 | 380.40 | 499.99 | 651.72 |
| (μL/min) | Dev. | | | | | | | | | | |
| | Min. | 165.95 | 303.67 | 319.36 | 316.05 | 108.91 | 577.69 | 469.69 | 1007.43 | 995.16 | 1274.55 |
| | Max. | 604.12 | 2119.47 | 1108.96 | 1191.97 | 1448.78 | 1472.15 | 2210.13 | 2479.33 | 2846.06 | 3483.00 |
| | N | 19 | 20 | 20 | 19 | 20 | 20 | 20 | 20 | 19 | 19 |
| Flow | Mean | 497.66 | 771.15 | 810.43 | 960.21 | 1188.57 | 1177.28 | 1626.63 | 2000.50 | 2345.60 | 2910.26 |
| Rate | Median | 540.05 | 744.1 | 917.54 | 1040.89 | 1281.73 | 1227.79 | 1737.56 | 2075.91 | 2567.52 | 3174.66 |
| (R² > | Std. | 116.09 | 353.06 | 241.71 | 224.56 | 271.12 | 238.38 | 488.49 | 380.40 | 499.99 | 651.72 |
| 0.98) | Dev. | | | | | | | | | | |
| (μL/min) | Min. | 165.95 | 303.67 | 319.36 | 316.05 | 416.71 | 577.69 | 469.69 | 1007.43 | 995.16 | 1274.55 |
| | Max. | 604.12 | 2119.47 | 1108.96 | 1191.97 | 1448.78 | 1472.15 | 2210.13 | 2479.33 | 2846.06 | 3483.00 |
| | N | 19 | 20 | 20 | 19 | 19 | 20 | 19 | 20 | 19 | 19 |

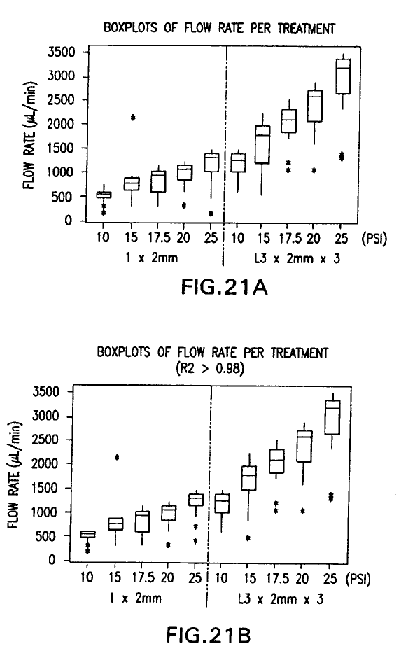

The distribution of flow rate measurements per treatment are shown in FIG. 21.

Flow rate (all values of R² and the subset of flow rates with R²>0.98) was analyzed using ANOVA. The ANOVA model included subject-to-subject differences, order of injection, device type and pressure main effects and device type by pressure interactions. Because of the non-constant variability/non-normality seen in the residuals, a log transformation was applied to the data and analyzed using ANOVA. The ANOVA results showed that both the device type and the pressure are significant, but their interaction were not indicated. The analyses of all flow rate data and of flow rate data with R²>0.98 were similar; the difference was in the tightness of the confidence intervals (flow rate data with R²>0.98 had narrower confidence intervals around differences). The following results were observed:

### Device Type:

The average flow rate for the L3×2 mm×3 device type was significantly higher by 143.9% (with 95% CI of (127.0%, 162.1%)) than the average flow rate for the 1×2 mm device type.
For flow rate data with R²>0.98, the average flow rate for the L3×2 mm×3 device type was significantly higher by 139.7% (with 95% CI of (125.2%, 155.1%)) than the average flow rate for the 1×2 mm device type.

### Pressure:

The average flow rate increased significantly and steadily by 128% (with 95% CI of (94.5%, 167.3%)) between the 10 PSI and 25 PSI pressures.
For flow rate data with R²>0.98, the average flow rate increased significantly and steadily by 138.6% (with 95% CI of (107.7%, 174.1%)) between the 10 PSI and 25 PSI pressures.

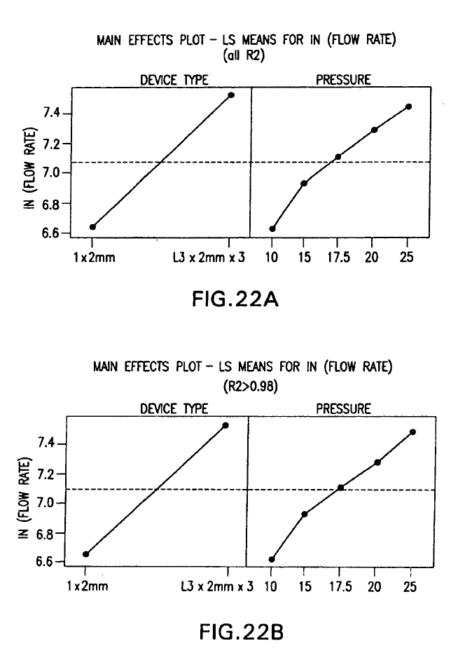

The size and magnitude of the significant device type and pressure effects in natural log scale are shown in FIG. 22.

6.3.9.2 Leakage

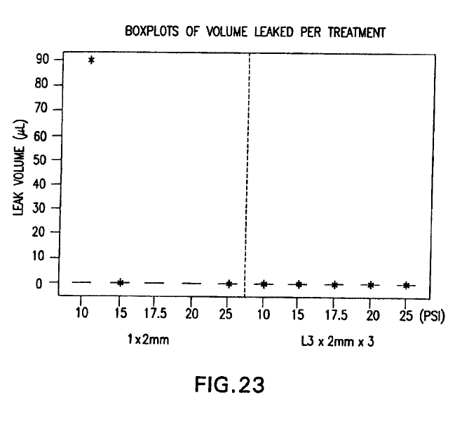

The actual recorded leakage volume is shown in FIG. 23. There was one occurrence of substantial leakage (treatment A, 1×2 mm, 10 PSI).

Table 25 summarizes the failures. With only one occurrence of failure to inject more than 95% of the intended injection volume there was no significant factor effect on the probability of leakage (with a sample size of 20 for each treatment condition, a difference of at least 10% in probability of failure was needed for 90% power of detection between the two device types, and a difference of at least 22% in probability of failure was needed for 90% power of detection between the different pressures). Individual 95% upper bounds on the probability of failing to inject at least 95% of 250 μl in the thigh were calculated for the various treatments. For those treatments with no occurrences of major leakage out of 20 infusions, the 95% upper bound on the probability of failing to inject at least 95% of 250 μl in the thigh is 13.9%. In other words, there is a 95% confidence that the chance of failing to inject at least 95% of the intended volume for treatments B-G & I is no more than 13.9%.

TABLE 25
Summary of Failures

| | | | | Individual |
|---|---|---|---|---|
| | | | | 95% upper bound |
| | | | Number | on Probability |
| | | | of Failures | of Failure to |
| | | | to inject at least | inject at least |
| Treatment | Device | PSI | 95% of 500 μL | 95% of 500 μL |
| A | 1 × 2 mm | 10 | 1/20 | 21.6% |
| B | L3 × 2 mm × 3 | 10 | 0/20 | 13.9% |
| C | 1 × 2 mm | 15 | 0/20 | 13.9% |
| D | L3 × 2 mm × 3 | 15 | 0/20 | 13.9% |
| E | 1 × 2 mm | 17.5 | 0/20 | 13.9% |
| F | L3 × 2 mm × 3 | 17.5 | 0/20 | 13.9% |
| G | 1 × 2 mm | 20 | 0/20 | 13.9% |
| H | L3 × 2 mm × 3 | 20 | 0/19 | 14.6% |
| I | 1 × 2 mm | 25 | 0/20 | 13.9% |
| J | L3 × 2 mm × 3 | 25 | 0/18 | 15.3% |

The failure summary for per factor level is provided in Table 26.

TABLE 26
Summary of Failures for per Factor Level

| | | | Individual 95% |
|---|---|---|---|
| | | | upper bound on |
| | | Number of | Probability of Failure |
| | | Failures to inject | to inject at |
| | | at least 95% of | least 95% |
| Factor | Levels | 250 μL | of 250 μL |
| Device Type | 1 × 2 mm | 1/100 | 4.7% |
| | L3 × 2 mm × 3 | 0/97 | 3.0% |
| Pressure | 10 | 1/40 | 11.3% |
| | 15 | 0/40 | 7.2% |
| | 17.5 | 0/40 | 7.2% |
| | 20 | 0/39 | 7.4% |
| | 25 | 0/38 | 7.6% |

6.3.9.3 Pain

Statistics of the overall pain are summarized in Table 27.

TABLE 27
Summary Statistics of Overall Pain

| | | Device | | | | | | | | | |
|---|---|---|---|---|---|---|---|---|---|---|---|
| | | 1 × 2 mm | | | | | L3 × 2 mm × 3 | | | | |
| | | 10 PSI | 15 PSI | 17.5 PSI | 20 PSI | 25 PSI | 10 PSI | 15 PSI | 17.5 PSI | 20 PSI | 25 PSI |
| Over- | Mean | 2.6 | 3.6 | 3.8 | 4.3 | 4.7 | 4.5 | 5.3 | 5.6 | 6.0 | 6.3 |
| all | Median | 2 | 2 | 4 | 4 | 3 | 3 | 4.5 | 4 | 5 | 6 |
| Pain | SD | 2.6 | 3.7 | 2.9 | 3.2 | 4.3 | 4.2 | 4.5 | 4.7 | 4.2 | 4.6 |
| | Min | 0 | 0 | 0 | 0 | 0 | 0 | 0 | 0 | 0 | 0 |
| | Max | 9 | 13 | 9 | 12 | 15 | 13 | 18 | 15 | 17 | 17 |
| | N | 19 | 20 | 20 | 19 | 20 | 20 | 20 | 20 | 20 | 19 |

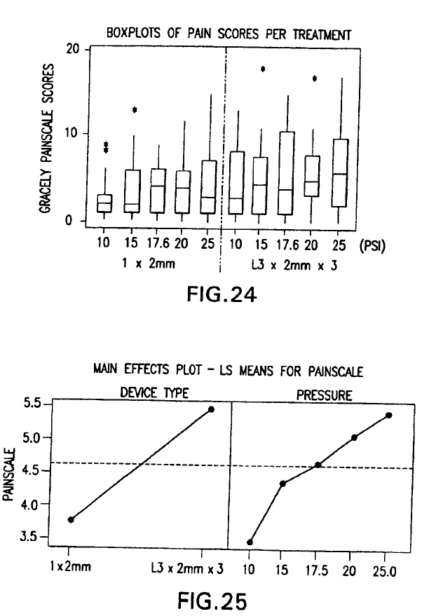

The distribution of pain scores is shown in FIG. 24.

Pain scores were analyzed using ANOVA. The ANOVA model included subject-to-subject differences, order of injection, device type and pressure main effects and device type by pressure interactions. The ANOVA results showed that both the device type and the pressure are significant, but their interaction was not indicated. The following results were observed:

### Device Type:

The average pain for the L3×2 mm×3 device type was significantly higher by 1.7 pain scale units (with 95% CI of (0.9, 2.5)) than the average pain for the 1×2 mm device type.

### Pressure:

The average pain increased significantly and steadily from an average pain score of 3.5 with 10 PSI to an average pain score of 5.4 with 25 PSI.

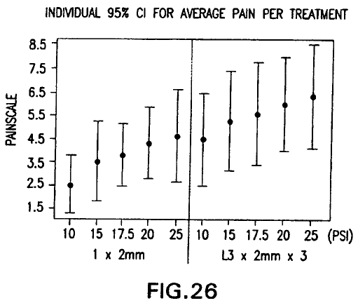

The size and magnitude of the significant device type and pressure effects are shown in FIG. 25, and individual confidence intervals for average pain per device are shown in FIG. 26.

6.3.9.4 Wheal

The number and percent wheals (given a successful injection) for each device are summarized in Table 28 below. A logistic regression was used to investigate the effect of device type and pressure on wheal formation and results showed that neither factor had a significant effect.

TABLE 28
Summary of Wheal Formation

| | Pressure | | 95% lower bound on |
|---|---|---|---|
| Device Type | (PSI) | Wheal | probability of wheal |
| 1 × 2 mm | 10 | 6/19 = 31.6% | 14.7% |
| | 15 | 7/20 = 35.0% | 17.7% |
| | 17.5 | 8/20 = 40.0% | 21.7% |
| | 20 | 9/20 = 45.0% | 25.9% |
| | 25 | 6/20 = 30.0% | 14.0% |
| L3 × 2 mm × 3 | 10 | 6/20 = 30.0% | 14.0% |
| | 15 | 7/20 = 35.0% | 17.7% |
| | 17.5 | 2/20 = 10.0% | 1.8% |
| | 20 | 5/20 = 25.0% | 10.4% |
| | 25 | 6/20 = 30.0% | 14.0% |

6.3.9.5 Effects on Leakage

Table 29 summarizes the number of times leakage was observed for each device and where the leakage was observed for all injections. A logistic regression was used to investigate the effect of device type and pressure on fluid seen and results showed that neither factor had a significant effect.

TABLE 29
Summary of Fluid Seen Upon Removal of Device

| | Pressure | Fluid Seen | | | |
|---|---|---|---|---|---|
| Device Type | (PSI) | Yes | Device | Skin | Both |
| 1 × 2 mm | 10 | 1/19 = 5.3% | 0 | 1 | 0 |
| | 15 | 3/20 = 15% | 1 | 2 | 0 |
| | 17.5 | 3/20 = 15% | 0 | 3 | 0 |
| | 20 | 3/19 = 15.8% | 0 | 2 | 1 |
| | 25 | 2/20 = 10% | 0 | 1 | 1 |
| L3 × 2 mm × 3 | 10 | 5/20 = 25% | 2 | 2 | 1 |
| | 15 | 3/20 = 15% | 1 | 2 | 0 |
| | 17.5 | 1/20 = 5% | 0 | 1 | 0 |
| | 20 | 6/20 = 30% | 3 | 1 | 2 |
| | 25 | 5/20 = 25% | 3 | 0 | 2 |

The cause of leakage for thirty one (31) of the above “fluid seen” responses was “weeping/pesky drop.” The remaining cause (L3×2 mm×3 device type, 25 PSI) was determined to be mechanical or adhesive failure.

6.3.9.6 Safety

Safety was assessed using the erythema and edema Draize scores, as described above. Table 30 shows the summary of the results.

TABLE 30
Summary of Erythema and Edema

| | | Erythema | | | Edema | | |
|---|---|---|---|---|---|---|---|
| Device | | | 1 | | | 1 | |
| Type | | 0 | Very | Total | 0 | Very | Total |
| Pressure | (PSI) | None | Slight | Erythema | None | Slight | Edema |
| 1 × 2 mm | 10 | 18 | 1 | 2/98 = | 18 | 1 | 9/98 = |
| | 15 | 20 | 0 | 2% | 16 | 4 | 9.2% |
| | 17.5 | 19 | 1 | | 18 | 2 | |
| | 20 | 19 | 0 | | 17 | 2 | |
| | 25 | 20 | 0 | | 20 | 0 | |
| L3 × | 10 | 15 | 5 | 15/99 = | 19 | 1 | 6/99 = |
| 2 mm × 3 | 15 | 17 | 3 | 15.2% | 18 | 2 | 6.1% |
| | 17.5 | 18 | 2 | | 19 | 1 | |
| | 20 | 18 | 2 | | 19 | 1 | |
| | 25 | 16 | 3 | | 18 | 1 | |

6.3.9.7 Summary

This 20-subjects study was performed to investigate the use of an air pressure mediated infusion system to effectively deliver fluid (250 μl) into the intradermal and shallow SC spaces using a constant pressure force (10, 15, 17.5, 20 and 25 psi) with the BD Micromedica single needle (1×2 mm) and Linear, three needle (L3×2 mm×3 mm spacing) devices. The following results were observed:

Flow rate: Both device type and pressure were significant. Average flow rate was higher for the L3×2 mm×3 device type than the average flow rate for the 1×2 mm device type. Average flow rate increased with pressure.

Leakage: One occurrence of substantial leakage (treatment A, 1×2 mm, 10 PSI) was observed, but no significant factor effects on the probability of leakage were observed.

Pain: Both device type and pressure were significant. Average pain was higher for the L3×2 mm×3 device type than the average pain for the 1×2 mm device type. Average pain increased with pressure.

Wheal: No significant device type or pressure effect was observed.

Fluid Seen Upon Removal of Device: No significant device type or pressure effect was observed.

Erythema and Edema: Significant device type effect on erythema was observed, with the L3×2 mm×3 device type resulting in more instances of very mild erythema.

6.3.10 Device Effects and Interactions

To investigate the main effects and interactions of the various factors encountered during constant pressure delivery, the following studies were designed: An air pressure mediated infusion system was used to deliver 500 μl of fluid or for five minutes, whichever comes first, into the intradermal and shallow SC spaces of subjects to determine whether the factors, such as infusion pressure, needle length, needle number and injection site, have an individual or combined effect. Not all combinations of factors are pertinent for the anticipated final microneedle delivery devices or anticipated therapies (e.g., delivery in the deltoid with 3 mm systems is unlikely for either vaccine or drug delivery). Likewise, complete investigation of all possible combinations or even utilizing a fractional factorial design would necessitate a substantial number of study subjects and/or a prohibitively large number of conditions per subject. To avoid this, the study design was broken up into two sub-experiments that are performed as incomplete block designs to reduce the estimate of the experimental variance, keep the design balanced, focus on the most pertinent combinations of expected final device configurations, and to incorporate past clinical learning on device functional similarities (e.g., (1 mm=1.5 mm)±(2 mm=3 mm)).

The sub-experiments were as follows:

Sub-Study 1:

| | |
|---|---|
| Infusion Pressure | 10, 15 and 20 psi |
| Needle Length | 1, 1.5 and 2 mm |
| Needle Number | Single needle and linear 3-needle array |
| Site | Thigh, abdomen and deltoid |

Full factorial replicated three times. Each full replication required 9 subjects (blocks) and each group of 9 subjects was confounded with a different interaction.

Sub-Study 2:

| | |
|---|---|
| Infusion Pressure | 10 and 20 psi |
| Needle Length | 2 and 3 mm |
| Needle Number | Single needle and linear 3-needle array |
| Site | Thigh and abdomen |

Full factorial replicated completely six times (some combinations replicated seven times). Each full replication required 4 subjects (blocks) and each group of 4 subjects was confounded with two different interactions.

A total of 27 subjects received up to 10 infusions of sterile non-bacteriostatic saline for injection. Because of the incomplete block design, different subjects received a different combination of study conditions. Sub-study 1 utilized each possible combination of factors 3 times and sub-study 2 utilized each possible combination of factors 6 or 7 times. The sample size for the current study design was based on the observed variability seen in a previous constant pressure trial and was anticipated to yield statistically significant results for main factor effects and interactions. If confidence intervals obtained with the initial sample were too wide to be conclusive, Stein's two-stage approach (Sample Size Methodology, M. M. Desu and D. Raghavarao, Academic Press (1990)) for sample size determination was used to calculate the number of additional subjects needed to reduce the width of the confidence interval to a specified precision.

6.3.10.1 Sub-Study 1

6.3.10.1.1 Major Leakage and Incomplete Injections

There were a total of 26 failed injections consisting of 7 major leakages and 19 incomplete injections with no major leakage. A binary logistic regression was used to determine whether any of the factors in sub-study 1 had a significant effect on failure. Results indicated the following:

Factors with a significant effect on failure:
Needle Length: Length of 1 mm had significantly more failures than lengths of 1.5 mm & 2.0 mm. No significant difference between lengths of 1.5 mm & 2.0 mm was observed.
Site by Pressure interaction: The abdomen had significantly more failures than thigh or deltoid at 20 psi.
Needle Length by Number of Needles interaction: For 1 mm needles, there were significantly more failures with the single needles than with the 3-needle arrays.

The following tables summarize the results above:

TABLE 31
Summary of Failures - Main effects

| | | Number and | | |
|---|---|---|---|---|
| | | % of Failures | | |
| | | to inject at | Number and % | Number and % of |
| | | least 90% of | of Major | Incomplete (with no |
| Effect | Levels | 500 μL | Leakages | major leakage) |
| Needle | 1.0 mm | 20/72 = 27.8% | 5/72 = 6.9% | 15/72 = 20.8% |
| Length | 1.5 mm | 4/72 = 5.6% | 2/72 = 2.8% | 2/72 = 2.8% |
| | 2.0 mm | 2/72 = 2.8% | 0/72 = 0% | 2/72 = 2.8% |

TABLE 32
Summary of Failures - Site by Pressure Interaction

| | | Number and % of | | | | | | Number and % of | | |
|---|---|---|---|---|---|---|---|---|---|---|
| | | Failures to inject at | | | Number and % of Major | | | Incomplete (with no | | |
| | | least 90% of 500 μL | | | Leakages | | | major leakage) | | |
| | | Pressure (psi) | | | Pressure (psi) | | | Pressure (psi) | | |
| | | 10 | 15 | 20 | 10 | 15 | 20 | 10 | 15 | 20 |
| Site | Abdomen | 4/24 = | 3/24 = | 7/24 = | 1/24 = | 2/24 = | 4/24 = | 3/24 = | 1/24 = | 3/24 = |
| | | 16.7% | 12.5% | 29.2% | 4.2% | 8.3% | 16.7% | 12.5% | 4.2% | 12.5% |
| | Deltoid | 4/24 = | 3/24 = | 0/24 = | 0/24 = | 0/24 = | 0/24 = | 4/24 = | 3/24 = | 0/24 = |
| | | 16.7% | 12.5% | 0% | 0% | 0% | 0% | 16.7% | 12.5% | 0% |
| | Thigh | 3/24 = | 1/24 = | 1/24 = | 0/24 = | 0/24 = | 0/24 = | 3/24 = | 1/24 = | 1/24 = |
| | | 12.5% | 4.2% | 4.2% | 0% | 0% | 0% | 12.5% | 4.2% | 4.2% |

TABLE 33
Summary of Failures - Needle Length by Number of Needles

| | | Number &% | | | | | |
|---|---|---|---|---|---|---|---|
| | | of Failures | | | | Number and % | |
| | | to inject | | Number and % | | of Incomplete | |
| | | at least 90% of | | of Major | | (with no | |
| | | 500 μL | | Leakages | | major leakage) | |
| | | Number of | | Number of | | Number of | |
| | | Needles | | Needles | | Needles | |
| | | 1 | 3 | 1 | 3 | 1 | 3 |
| Needle | 1.0 | 15/36 = | 5/35 = | 3/36 = | 2/36 = | 12/36 = | 3/36 = |
| Length | mm | 41.7% | 13.9% | 8.3% | 5.6% | 33.3% | 8.3% |
| | 1.5 | 2/36 = | 2/36 = | 1/36 = | 1/36 = | 1/36 = | 1/36 = |
| | mm | 5.6% | 5.6% | 2.8% | 2.8% | 2.8% | 2.8% |
| | 2.0 | 0/36 = | 2/36 = | 0/36 = | 0/36 = | 0/36 = | 2/36 = |
| | mm | 0% | 5.6% | 0% | 0% | 0% | 5.6% |

6.3.10.1.2 Flow Rate

Summary statistics of flow rate measurements per factor level (successful injections only) are shown in Table 34 below.

TABLE 34
Data Summary Statistics for Flow Rate

| | | Statistic | | | | | |
|---|---|---|---|---|---|---|---|
| | | | | | | | N (number of |
| Factor | Levels | Mean | Median | SD | Min | Max | replicates) |
| Number of | 1 needle | 490.967 | 474.94 | 231.605 | 106.45 | 1176.72 | 91 |
| Needles | 3 needles | 989.905 | 889.58 | 609.359 | 106.06 | 2741.03 | 99 |
| Needle | 1.0 mm | 548.472 | 411.455 | 422.755 | 106.06 | 2111.47 | 52 |
| Length | 1.5 mm | 716.889 | 581.820 | 492.305 | 114.54 | 2405.26 | 68 |
| | 2.0 mm | 934.422 | 772.825 | 578.606 | 106.45 | 2741.03 | 70 |
| Site | Abdomen | 688.280 | 642.83 | 407.804 | 120.82 | 2154.73 | 58 |
| | Deltoid | 597.468 | 474.94 | 435.755 | 106.06 | 1936.57 | 65 |
| | Thigh | 954.074 | 760.48 | 638.005 | 130.08 | 2741.03 | 67 |
| Pressure | 10 psi | 529.384 | 451.980 | 320.242 | 106.06 | 1533.35 | 61 |
| | 15 psi | 761.519 | 663.870 | 485.031 | 106.92 | 2386.34 | 65 |
| | 20 psi | 951.366 | 774.805 | 645.355 | 106.45 | 2741.03 | 64 |

Summary statistics of flow rate measurements per factor level for the subset of flow rates with R²>0.98 are shown in Table 35 below.

TABLE 35
Data Summary Statistics for Flow Rate with R² > 0.98

| | | Statistic | | | | | |
|---|---|---|---|---|---|---|---|
| | | | | | | | N (number of |
| Factor | Levels | Mean | Median | SD | Min | Max | replicates) |
| Number of | 1 needle | 495.080 | 483.15 | 229.537 | 106.45 | 1176.72 | 90 |
| Needles | 3 needles | 997.941 | 896.25 | 607.196 | 106.06 | 2741.03 | 98 |
| Needle | 1.0 mm | 548.472 | 411.455 | 422.755 | 106.06 | 2111.47 | 52 |
| Length | 1.5 mm | 725.786 | 582.950 | 490.483 | 114.54 | 2405.26 | 67 |
| | 2.0 mm | 945.032 | 778.700 | 575.945 | 106.45 | 2741.03 | 69 |
| Site | Abdomen | 707.090 | 647.18 | 402.298 | 123.86 | 2154.73 | 56 |
| | Deltoid | 597.468 | 474.94 | 435.755 | 106.06 | 1936.57 | 65 |
| | Thigh | 954.074 | 760.48 | 638.005 | 130.08 | 2741.03 | 67 |
| Pressure | 10 psi | 534.834 | 459.045 | 320.078 | 106.06 | 1533.35 | 60 |
| | 15 psi | 761.519 | 663.870 | 485.031 | 106.92 | 2386.34 | 65 |
| | 20 psi | 964.549 | 777.820 | 641.793 | 106.45 | 2741.03 | 63 |

Flow rate (all values of R² and the subset of flow rates with R²>0.98) was analyzed using ANOVA. The ANOVA model included subject-to-subject differences, order of injection, site, needle length, number of needles and pressure main effects and 2-way factor interactions. Because of the non-constant variability/non-normality seen in the residuals, a log transformation was applied to the data and analyzed using ANOVA. The ANOVA results showed that for flow rate data (including all values of R²), all of the factors examined were significant and the site by number of needles interaction was also significant. For flow rate data with R²>0.98, the pressure by site interaction was also significant. Factors with a significant effect on flow rate (successful injection only), in decreasing order of significance, were: needle number; needle length; pressure; site; site by needle number; and pressure by site (for the subset of data with R²>0.98).

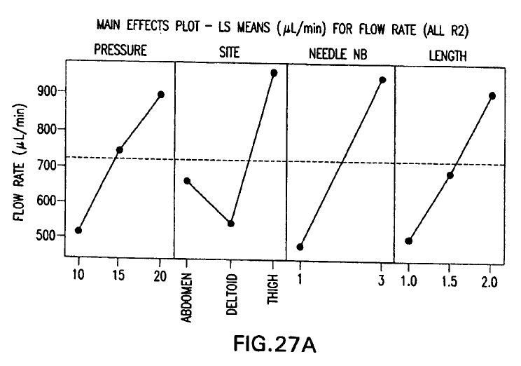

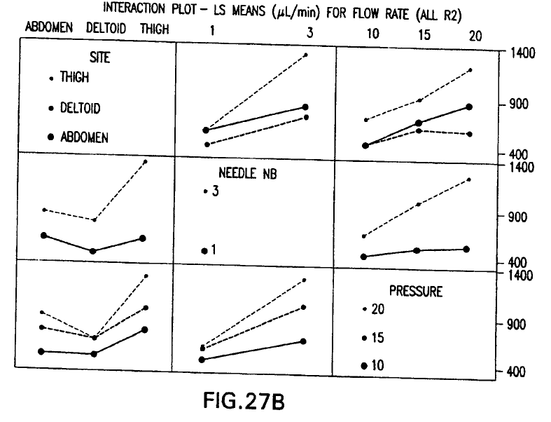

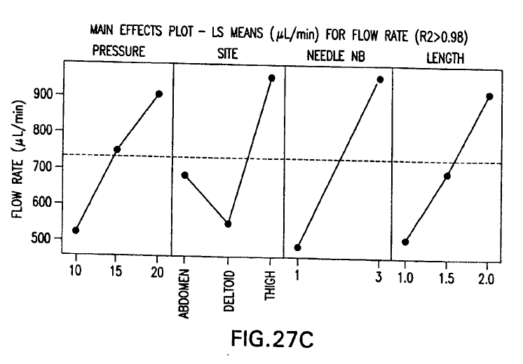

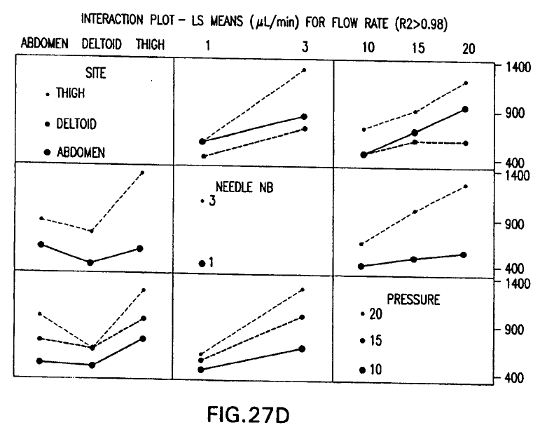

The size and magnitude of the significant effects and interaction in μl/min are shown in FIG. 27 (approximate values due to the fact that they are back transformed into original units from the least-squares means predicted from the fitted ANOVA model which used logged data). Main effects in percent difference for flow rate, for the subset of data with R²>0.98, are shown in Table 36 below.

TABLE 36
Main Effect in % Difference for Flow Rate

| | | Estimated % | |
|---|---|---|---|
| Factor | Main Effect | Difference | 95% Confidence Interval |
| Number of | 3 needles − 1 needle | 102.16% | (79.14, 128.17) |
| Needles | | | |
| Needle | 1.5 mm − 1.0 mm | 76.00% | (45.72, 112.57) |
| Length | 2.0 mm − 1.0 mm | 134.60% | (94.59, 182.81) |
| | 2.0 mm − 1.5 mm | 33.30% | (12.75, 57.59) |
| Site | Abdomen − Deltoid | 31.93% | (18.28, 43.31) |
| | Abdomen − Thigh | −13.97% | (−36.44, 4.79) |
| | Deltoid − Thigh | 67.45% | (40.99, 98.87) |
| Pressure | 15 psi − 10 psi | 47.93% | (23.64, 77.00) |
| | 20 psi − 10 psi | 92.03% | (60.16, 130.23) |
| | 20 psi − 15 psi | 29.81% | (8.72, 54.98) |

Site by needle number interaction and pressure by site interaction in percent difference, for the subset of data with R²>0.98, are shown in Tables 37 and 38 below.

TABLE 37
Site by Needle Number Interaction in % Difference

| | | Estimated % Difference |
|---|---|---|
| | | (with 95% Confidence interval) |
| Factor | Level | 3 needles − 1 needle |
| Site | Abdomen | 59.62% |
| | | (16.02, 119.61) |
| | Deltoid | 94.31% |
| | | (44.80, 160.75) |
| | Thigh | 166.42% |
| | | (99.01, 256.80) |

TABLE 38
Pressure by Site Interaction in % Difference

| | | Estimated % Difference | | |
|---|---|---|---|---|
| | | (with 95% Confidence interval) | | |
| Factor | Level | 15 psi − 10 psi | 20 psi − 10 psi | 20 psi − 15 psi |
| Site | Abdomen | 1.63% | 27.44% | 25.40% |
| | | (−42.31, 78.98) | (−29.39, | (−31.00, 127.87) |
| | | | 130.06) | |
| | Deltoid | 36.97% | 80.51% | 31.78% |
| | | (−23.75, | (3.24, 215.60) | (−25.20, 132.17) |
| | | 146.06) | | |
| | Thigh | 132.57% | 207.81% | 32.35% |
| | | (32.33, 308.74) | (73.78, 445.26) | (−23.67, 129.49) |

6.3.10.1.3 Pain

Summary statistics for overall perceived pain per factor level (successful injections only) are shown in Table 39 below.

TABLE 39
Data Summary Statistics for Pain

| | | Statistic | | | | | |
|---|---|---|---|---|---|---|---|
| | | | | | | | N |
| | | | | | | | (number of |
| Factor | Levels | Mean | Median | SD | Min | Max | replicates) |
| Number | 1 needle | 5.0 | 5 | 3.9 | 0 | 18 | 91 |
| of | 3 needles | 6.4 | 6 | 4.7 | 0 | 19 | 99 |
| Needles | | | | | | | |
| Needle | 1.0 mm | 6.5 | 6 | 4.1 | 0 | 16 | 52 |
| Length | 1.5 mm | 6.4 | 6 | 4.6 | 0 | 19 | 68 |
| | 2.0 mm | 4.5 | 4 | 4.1 | 0 | 15 | 70 |
| Site | Abdomen | 6.6 | 7 | 4.2 | 0 | 15 | 58 |
| | Deltoid | 5.8 | 5 | 4.8 | 0 | 19 | 65 |
| | Thigh | 4.9 | 5 | 3.9 | 0 | 16 | 67 |
| Pressure | 10 psi | 5.1 | 5 | 3.9 | 0 | 17 | 61 |
| | 15 psi | 5.6 | 5 | 4.5 | 0 | 19 | 65 |
| | 20 psi | 6.4 | 6 | 4.6 | 0 | 17 | 64 |

Pain scores were analyzed using ANOVA. The ANOVA model included subject-to-subject differences, order of injection, site, needle length, number of needles and pressure main effects and 2-way factor interactions. The ANOVA results showed that all factors were significant and the pressure by number of needles interaction was also significant. Factors with a significant effect on pain (successful injection only), in decreasing order of significance, were: needle number; needle length; site; pressure; and pressure by needle number.

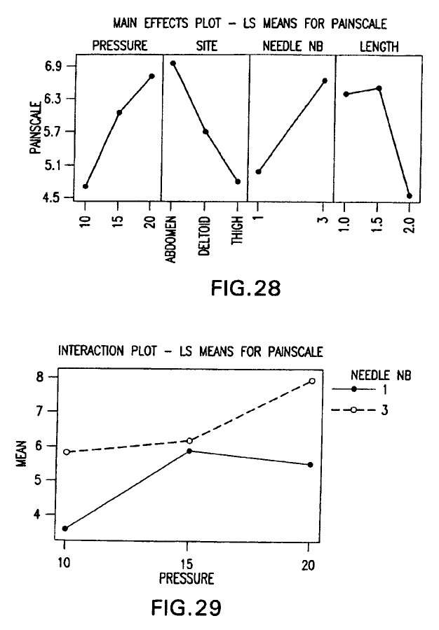

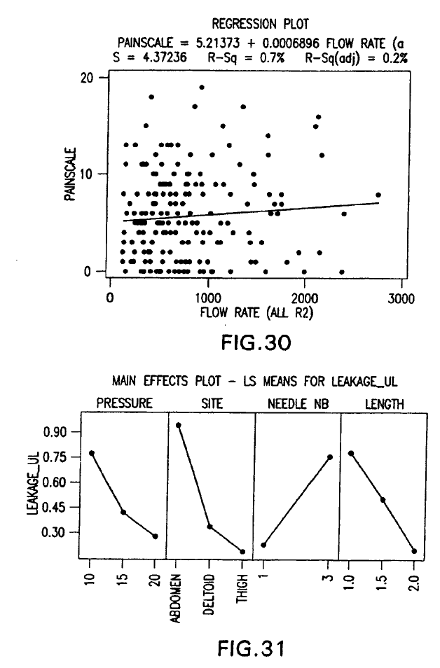

The size and magnitude of the significant main effects are shown in FIG. 28; the pressure by number of needles interaction is shown in FIG. 29; and pain and flow rate correlation is illustrated in FIG. 30. As shown in FIG. 30, no significant relationship between pain and flow rate was observed.

6.3.10.1.4 Wheal

A binary logistic regression was used to determine whether any of the factors in the sub-study 1 had a significant effect on wheal. Results indicated that needle length and site had an impact on wheal formation. With regard to needle length, length of 2.0 mm had significantly fewer wheals than lengths of 1.5 mm & 11.0 mm, but no significant difference between lengths of 1.5 mm & 11.0 mm was observed. As for the site, there was a significant difference between all sites. The site with fewest wheals was thigh, followed by the abdomen and then deltoid. The following table summarizes number and percent wheals (given a successful injection) for the significant main effects.

TABLE 40
Summary of Wheals - Main effects

| Effect | Levels | Number &% of Wheals |
|---|---|---|
| Needle Length | 1.0 mm | 50/52 = 96.2% |
| | 1.5 mm | 63/68 = 92.7% |
| | 2.0 mm | 54/70 = 77.1% |
| Site | Abdomen | 54/58 = 93.1% |
| | Deltoid | 65/65 = 100% |
| | Thigh | 48/67 = 71.6% |

6.3.10.1.5 Leakage

A binary logistic regression was used to determine whether any of the factors in sub-study 1 had a significant effect on fluid seen upon removal of the device (given a successful injection). Results indicated that number of needles and site had an impact on leakage. With regard to the number of needles, the 3-needles device had significantly more occurrences of fluid observed upon removal of the device than the single needle device. As for the site, abdomen had significantly more occurrences of fluid than thigh or deltoid. The following table summarizes number and percent of successful injections with fluid seen upon removal of device and where the fluid was seen for the significant main effects:

TABLE 41
Summary of Fluid Seen Upon Removal of the Device

| | | Number and % of | | | Device & |
|---|---|---|---|---|---|
| Effect | Levels | injections with fluid | Skin | Device | Skin |
| Number of | 1 needle | 14/91 = 15.4% | 3 | 9 | 2 |
| Needles | 3 needles | 30/99 = 30.3% | 2 | 21 | 7 |
| Site | Abdomen | 22/58 = 37.9% | 2 | 15 | 5 |
| | Deltoid | 10/65 = 15.4% | 1 | 8 | 1 |
| | Thigh | 12/67 = 17.9% | 2 | 7 | 3 |

The cause of leakage for forty-four (44) of the above “fluid seen” responses was “weeping/pesky drop.” There was one occurrence marked as “mechanical failure/adhesive failure” (for L3×1.0 mm device, 10 PSI in the thigh), and two occurrences were marked as “mechanical failure/fluid path failure” (for L3×1.0 mm device, 10 PSI in the abdomen and single needle×1.5 mm device, 20 PSI in the thigh).

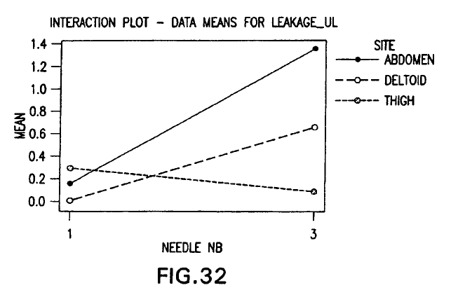

Leakage volume for injections with no major leakage was analyzed using ANOVA. The Anova was performed on transformed data because of the lack of normality in the residuals. The ANOVA model included subject-to-subject differences, order of injection, site, needle length, number of needles and pressure main effects and 2-way factor interactions. The ANOVA results showed that the number of needles and site were significant and the site×number of needles interaction was also significant. The size and magnitude of the significant main effects and interactions are shown in FIGS. 31 and 32.

6.3.10.1.7 Safety

Safety was assessed using the erythema and edema Draize scores, as described above. A binary or ordinal logistic regression was used to determine whether any of the factors in sub-study 1 had a significant effect on Draize scores. Results indicated significant needle length and site effects on edema. As the needle length increases, there is a tendency for edema to decrease. The thigh has significantly lower draize scores for edema. No significant effects were observed on erythema. Table 42 shows the summary of the edema scores.

TABLE 42
Summary of Edema Scores - Main Effect

| | | Edema Score | | |
|---|---|---|---|---|
| | | 0 | 1 | 2 |
| | | No edema | Very slight edema | Slight edema |
| Needle | 1.0 mm | 35/52 = 67.3% | 15/52 = 28.9% | 2/52 = 3.9% |
| Length | 1.5 mm | 53/68 = 77.9% | 14/68 = 20.6% | 1/68 = 1.5% |
| | 2.0 mm | 61/70 = 87.1% | 9/70 = 12.9% | 0/70 = 0% |
| Site | Abdomen | 39/58 = 67.2% | 18/58 = 31.0% | 1/58 = 1.7% |
| | Deltoid | 48/65 = 73.9% | 15/65 = 23.1% | 2/65 = 3.1% |
| | Thigh | 62/67 = 92.5% | 5/67 = 7.5% | 0/67 = 0% |

6.3.10.2 Sub-Study 2

6.3.10.2.1 Major Leakage and Incomplete Injections

There was a total of 5 failed injections, all incomplete injections with no major leakage. A binary logistic regression was used to determine whether any of the factors in sub-study 2 had a significant effect on failure. Results indicated that site of injection had an impact on failure, with abdomen having had significantly more failures than thigh. The results are summarized in Table 43.

TABLE 43
Summary of Failures - Main effects

| | | Number | | Number |
|---|---|---|---|---|
| | | and % of | | &% of |
| | | Failures to | | Incomplete |
| | | inject at least | Number &% of | (with no |
| Effect | Levels | 90% of 500 μL | Major Leakages | major leakage) |
| Site | Abdomen | 5/71 = 7.0% | 0% | 5/71 = 7.0% |
| | Thigh | 0/73 = 0% | 0% | 0% |

6.3.10.2.2 Flow Rate

Summary statistics of flow rate measurements per factor level (successful injections only) are shown in Table 44 below.

TABLE 44
Data Summary Statistics for Flow Rate

| | | Statistic | | | | | |
|---|---|---|---|---|---|---|---|
| | | | | | | | N |
| | | | | | | | (number of |
| Factor | Levels | Mean | Median | SD | Min | Max | replicates) |
| Number of | 1 needle | 670.90 | 589.24 | 270.020 | 81.01 | 1211.06 | 70 |
| Needles | 3 needles | 1617.61 | 1494.45 | 828.794 | 160.32 | 2998.60 | 68 |
| Needle | 2.0 mm | 1120.42 | 894.630 | 782.758 | 173.44 | 2998.60 | 70 |
| Length | 3.0 mm | 1154.87 | 968.685 | 769.279 | 81.01 | 2673.83 | 68 |
| Site | Abdomen | 938.49 | 728.79 | 696.802 | 81.01 | 2673.83 | 66 |
| | Thigh | 1319.72 | 1111.61 | 799.680 | 180.32 | 2998.60 | 72 |
| Pressure | 10 psi | 715.10 | 559.27 | 383.421 | 81.01 | 1564.84 | 69 |
| | 20 psi | 1559.69 | 1166.20 | 834.558 | 175.15 | 2998.60 | 69 |

Summary statistics of flow rate measurements per factor level for the subset of flow rates with R²>0.98 are shown in Table 45 below.

TABLE 45
Data Summary Statistics for Flow Rate with R² > 0.98

| | | Statistic | | | | | |
|---|---|---|---|---|---|---|---|
| | | | | | | | N |
| | | | | | | | (number of |
| Factor | Levels | Mean | Median | SD | Min | Max | replicates) |
| Number of | 1 needle | 679.44 | 590.31 | 262.282 | 180.32 | 1211.06 | 69 |
| Needles | 3 needles | 1707.85 | 1580.51 | 768.032 | 227.24 | 2998.60 | 64 |
| Needle | 2.0 mm | 749.86 | 584.48 | 367.322 | 180.32 | 1564.84 | 65 |
| Length | 3.0 mm | 1580.05 | 1181.09 | 823.317 | 227.24 | 2998.60 | 68 |
| Site | Abdomen | 1002.69 | 762.02 | 685.971 | 227.24 | 2673.83 | 61 |
| | Thigh | 1319.72 | 1111.61 | 799.680 | 180.32 | 2998.60 | 72 |
| Pressure | 10 psi | 749.86 | 584.48 | 367.322 | 180.32 | 1564.84 | 65 |
| | 20 psi | 1580.05 | 1181.09 | 823.317 | 227.24 | 2998.60 | 68 |

Flow rate (all values of R² and the subset of flow rates with R²>0.98) was analyzed using ANOVA. The ANOVA model included subject-to-subject differences, order of injection, site, needle length, number of needles and pressure main effects and 2-way factor interactions. Because of the non-constant variability/non-normality seen in the residuals, a log transformation was applied to the data and analyzed using ANOVA. The ANOVA results showed that for flow rate data (including all values of R²), all of the factors examined were significant and the site by number of needles interaction was also significant. For flow rate data with R²>0.98, the pressure by site interaction was also significant. Factors with a significant effect on flow rate (successful injection only), in decreasing order of significance, were: pressure; needle number; site; site by needle number (for data including all R²); and site by needle length (for the subset of data with R²>0.98).

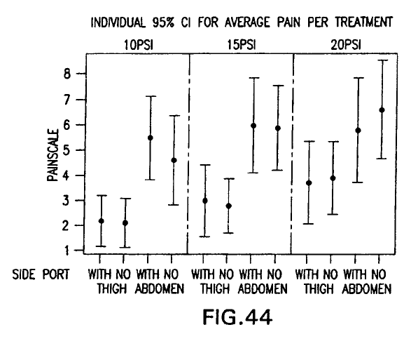

The size and magnitude of the significant effects and interaction in μl/min are shown in FIG. 44 (approximate values due to the fact that they are back transformed into original units from the least-squares means predicted from the fitted ANOVA model which used logged data). Main effects in percent difference for flow rate, for the subset of data with R²>0.98, are shown in Table 46 below.

TABLE 46
Main Effect in % Difference for Flow Rate

| | | Estimated % | 95% Confidence |
|---|---|---|---|
| Factor | Main Effect | Difference | Interval |
| Number of | 3 needles − 1 needle | 122.89% | (97.53, 151.48) |
| Needles | | | |
| Pressure | 20 psi − 10 psi | 97.19% | (74.19, 123.22) |
| Site | Thigh − Abdomen | 27.43% | (12.69, 44.11) |
| Length | 3.0 mm − 2.0 mm | 5.57% | (−6.41, 19.10) |

Site by needle number interaction, for data including all R², and pressure by site interaction in percent difference, for the subset of data with R²>0.98, are shown in Tables 47 and 48 below.

TABLE 47
Site by Needle Number Interaction

| | | Estimated % Difference |
|---|---|---|
| | | (with 95% Confidence Interval) |
| Factor | Level | 3 needles − 1 needle |
| Site | Abdomen | 68.66% |
| | | (22.37, 132.47) |
| | Thigh | 151.76% |
| | | (84.91, 242.81) |

TABLE 48
Site by Needle Length Interaction

| | | Estimated % Difference |
|---|---|---|
| | | (with 95% Confidence Interval) |
| Factor | Level | 3.0 mm − 2.0 mm |
| Site | Abdomen | 30.19% |
| | | (1.50, 66.98) |
| | Thigh | −14.38% |
| | | (−31.78, 7.46) |

6.3.10.2.3 Pain

Summary statistics for overall perceived pain per factor level (successful injections only) are shown below in Table 49.

TABLE 49
Data Summary Statistics for Pain

| | | Statistic | | | | | |
|---|---|---|---|---|---|---|---|
| | | | | | | | N |
| | | | | | | | (number of |
| Factor | Levels | Mean | Median | SD | Min | Max | replicates) |
| Number | 1 needle | 3.3 | 3 | 3.6 | 0 | 18 | 70 |
| of | 3 needles | 4.3 | 4 | 3.7 | 0 | 13 | 69 |
| Needles | | | | | | | |
| Needle | 2.0 mm | 4.3 | 4 | 4.0 | 0 | 18 | 70 |
| Length | 3.0 mm | 3.3 | 2 | 3.4 | 0 | 11 | 69 |
| Site | Abdomen | 4.7 | 4 | 4.2 | 0 | 18 | 66 |
| | Thigh | 3.1 | 2 | 3.1 | 0 | 11 | 73 |
| Pressure | 10 psi | 3.4 | 3 | 3.3 | 0 | 10 | 70 |
| | 20 psi | 4.2 | 3 | 4.1 | 0 | 18 | 69 |

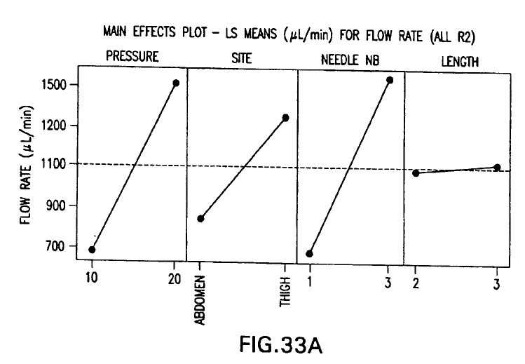

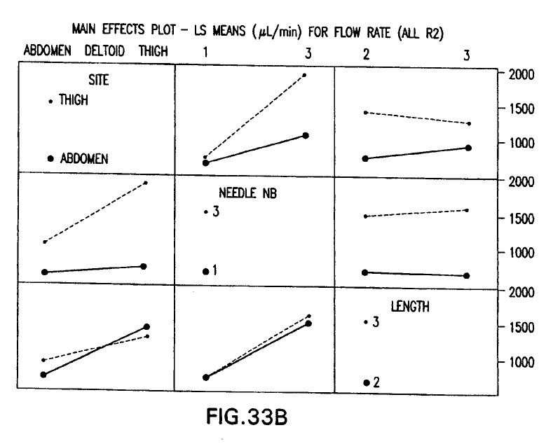

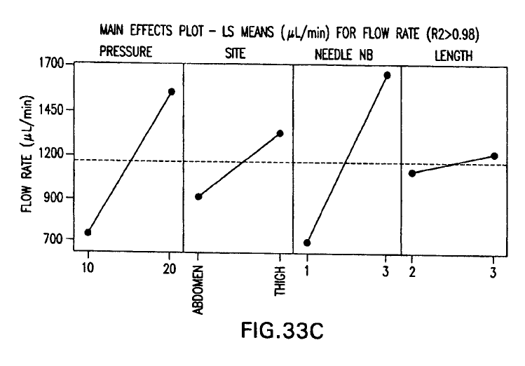

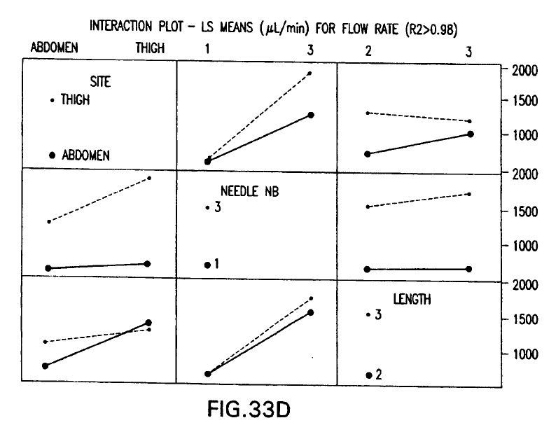

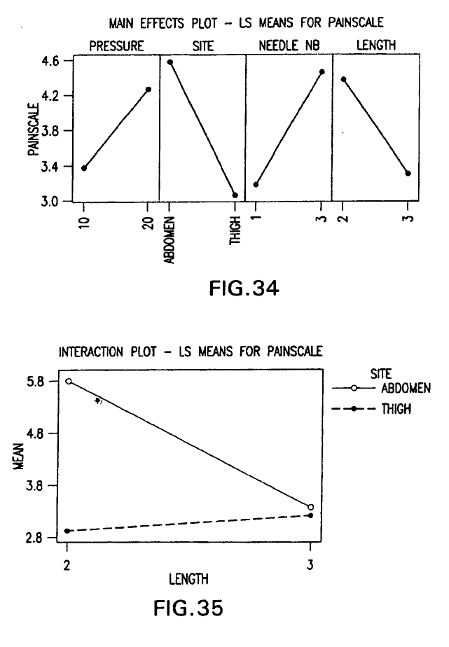

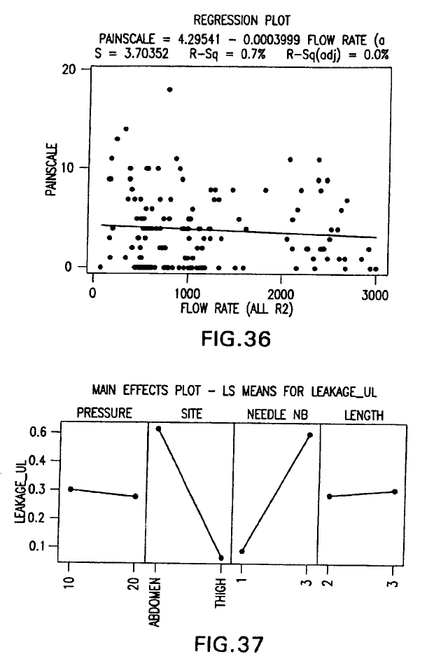

Pain scores were analyzed using ANOVA. The ANOVA model included subject-to-subject differences, order of injection, site, needle length, number of needles and pressure main effects and 2-way factor interactions. The ANOVA results showed that all factors were significant, and the site by needle length interaction was also significant. Factors with a significant effect on pain, in decreasing order of significance, were: site; needle number; site by needle length interaction; needle length; and pressure. The size and magnitude of the significant main effects are shown in FIGS. 33 and 34, and the site by needle length interaction is shown in FIG. 35. In addition, as shown in FIG. 36, no significant relationship between flow rate and pain was observed.

6.3.10.2.4 Wheal

A binary logistic regression was used to determine whether any of the factors in sub-study 2 had a significant effect on wheal. Results indicated that factors such as needle length, site and site by needle length interaction had an impact on wheal formation. With regard to needle length, length of 3.0 mm resulted in significantly fewer wheals than length of 2.0 mm. As for the site, thigh had significantly fewer wheals than abdomen. In addition, the needle length difference was only significant in the abdomen, and the site effect only significant with the 2.0 mm needle length. The 2.0 mm needle length in the abdomen had significantly more wheals than any of the other needle length by site combinations. The following tables summarize number and percent wheals (given a successful injection) for the significant main effects and interactions:

TABLE 50
Summary of Wheals - Main effects

| Effect | Levels | Number and % of Wheals |
|---|---|---|
| Needle Length | 2.0 mm | 34/70 = 48.6% |
| | 3.0 mm | 14/69 = 20.3% |
| Site | Abdomen | 30/66 = 45.5% |
| | Thigh | 18/73 = 24.7% |

TABLE 51
Summary of Wheals - Site by Needle Lengths interaction

Number and % of Wheals
Needle Length

| | | 2.0 mm | 3.0 mm |
|---|---|---|---|
| Site | Abdomen | 25/34 = 73.5% | 5/32 = 15.6% |
| | Thigh | 9/36 = 25.0% | 9/37 = 24.3% |

6.3.10.2.5 Leakage

A binary logistic regression was used to determine whether any of the factors in sub-study 2 had a significant effect on fluid seen upon removal of the device (given a successful injection). Results indicated that number of needles had an impact on leakage, with the 3-needles device having had significantly more occurrences of fluid than the single needle device. The following table summarizes number and percent of successful injections with fluid seen upon removal of device and where the fluid was seen for the significant main effects:

TABLE 52
Summary of Fluid Seen Upon Removal of the Device

| | | Number &% | | | |
|---|---|---|---|---|---|
| | | of injections | | | Device & |
| Effect | Levels | with fluid | Skin | Device | Skin |
| Number of | 1 needle | 7/70 = 10.0% | 3 | 3 | 1 |
| Needles | 3 needles | 22/69 = 31.9% | 6 | 11 | 5 |

The cause of leakage for twenty-nine (29) of the above “fluid seen” responses was “weeping/pesky drop.” There was one occurrence (for L3×3.0 mm device. 10 PSI in the abdomen) also marked as all of the following: “mechanical failure/short needle,” “mechanical failure/bent needle,” “mechanical failure/adhesive failure” and “mechanical failure/fluid Path failure.”

Leakage volume for injections with no major leakage was analyzed using ANOVA. The ANOVA was performed on transformed data because of the lack of normality in the residuals. The ANOVA model included subject-to-subject differences, order of injection, site, needle length, number of needles and pressure main effects and 2-way factor interactions. The ANOVA results showed that the number of needles and site were significant. The size and magnitude of the significant main effects are shown in FIG. 37.

6.3.10.2.7 Safety

A binary logistic regression was used to determine whether any of the factors in sub-study 2 had a significant effect on Draize scores. Results indicated that no significant factors were present for edema, but number of needles and number of needles by length interaction were significant for erythema. The number of needle effect appears only significant with the 2.0 mm length, with the Draize scores for erythema are lower with the single needle than the 3-needles device for 2.0 mm needles. The following table summarizes eryhema scores (given a successful injection) for the significant interaction.

TABLE 53
Summary of Erythema Scores - Main Effect

| | | Erythema Score | | | |
|---|---|---|---|---|---|
| | | Needle Length | | | |
| | | 2.0 mm | | 3.0 mm | |
| | | | 1 | 0 | 1 |
| | | 0 | Very slight | No | Very slight |
| | | No erythema | erythema | erythema | erythema |
| Number | 1 | 35/35 = 100% | 0/35 = 0% | 32/35 = | 3/35 = |
| of | | | | 91.4% | 8.6% |
| Needles | 3 | 29/35 = 82.9% | 6/37 = 17.1% | 30/34 = | 4/34 = |
| | | | | 88.2% | 11.8% |

6.3.10.3 Summary

This 36-subjects incomplete block design study was performed as two sub-studies to investigate the main effects and interactions of the following factors on flow rate and success of injection, encountered during constant pressure delivery. Tables below summarize the significant factors (either as a main effect or through an interaction) with an “X” in the cell.

TABLE 54
Summary of significant effects - Sub Study 1

| | | | |
|---|---|---|---|
| Needle | | Infusion | |
| Length | Site | Pressure | Needle Number |

Sub Study 1

| | | | | |
|---|---|---|---|---|
| Success of | X | X | X | X |
| Injection | (Main effect & | (Interaction with | (Interaction with Site) | (Interaction with Needle |
| (inject at least | Interaction with | Pressure) | | Length) |
| 90% of 500 μL) | | Needle Number) | | |

Sub Study 1 successful injections only

| | | | | |
|---|---|---|---|---|
| Flow rate | X | X | X | X |
| | (Main effect) | (Main effect & | (Main effect & | (Main effect &Interaction |
| | | Interaction with | Interaction with Site | with Site) |
| | | Needle Number) | when R² > .98) | |
| | | (Interaction with | | |
| | | Pressure when R² > .98) | | |
| Pain | X | X | X | X |
| | (Main effect) | (Main effect) | (Main effect & | (Main effect &Interaction |
| | | | Interaction with Needle | with Pressure) |
| | | | Number) | |
| Wheal | X | X | | |
| Formation | (Main effect) | (Main effect) | | |
| Fluid | | X | | X |
| | | (Main effect) | | (Main effect) |
| Bleeding | | | X | X |
| | | | (Interaction with Needle | (Main effect &Interaction |
| | | | Number) | with Pressure) |

| | | | |
|---|---|---|---|
| Draize | Edema | X | X |
| | | (Main effect) | (Main effect) |
| | Erythema | | |

TABLE 55
Summary of significant effects - Sub Study 2

| | | | |
|---|---|---|---|
| Needle | | Infusion | |
| Length | Site | Pressure | Needle Number |

Sub Study 2

| | |
|---|---|
| Success of | X |
| Injection | (Main effect) |
| (inject at least 90% | |
| of 500 μL) | |

Sub Study 2 successful injections only

| | | | | |
|---|---|---|---|---|
| Flow rate | X | X | X | X |
| | (Interaction with | (Main effect) | (Main effect) | (Main effect) |
| | Site when R² > .98) | (Interaction with | | (Interaction with Site for |
| | | Needle Length when | | all R²) |
| | | R² > .98) | | |
| | | (Interaction with | | |
| | | Needle Number for | | |
| | | all R²) | | |
| Pain | X | X | X | X |
| | (Main effect & | (Main effect & | (Main effect) | (Main effect) |
| | interaction with | interaction with | | |
| | Site) | Needle Length) | | |
| Wheal | X | X | | |
| Formation | (Main effect & | (Main effect & | | |
| | interaction with | interaction with | | |
| | Site) | Needle Length) | | |
| Fluid | | | | X |
| | | | | (Main effect) |
| Bleeding | | X | | |
| | | (Main effect) | | |

| | | | |
|---|---|---|---|
| Draize | Edema | | |
| | Erythema | X | X |
| | | (interaction with | (Main effect &interaction |
| | | Needle Number) | with Needle Length) |

6.3.11 34G Side-Ported Needle in Constant Pressure Infusion

To investigate the effects of side-ported needle as compared to plain catheters, following studies were designed: A total of 24 subjects received up to 12 infusions of sterile non-bacteriostatic saline for injection at different sites in the thigh and abdomen using investigational devices according to the parameters shown in Table 56 below.

TABLE 56
Parameters for 34G Side Ported Needle

| | | | Volume | | |
|---|---|---|---|---|---|
| Treatment | Device | Pressure | (ul) | Site | Wait time |
| A | 1 × 1.5 with side | 10 | 500 | Thigh | 1 min |
| | port | | | | |
| B | 1 × 1.5 without | 10 | 500 | Thigh | 1 min |
| | side port | | | | |
| C | 1 × 1.5 with side | 10 | 500 | Abdomen | 1 min |
| | port | | | | |
| D | 1 × 1.5 without | 10 | 500 | Abdomen | 1 min |
| | side port | | | | |
| E | 1 × 1.5 with side | 15 | 500 | Thigh | 1 min |
| | port | | | | |
| F | 1 × 1.5 without | 15 | 500 | Thigh | 1 min |
| | side port | | | | |
| G | 1 × 1.5 with side | 15 | 500 | Abdomen | 1 min |
| | port | | | | |
| HJ | 1 × 1.5 without | 15 | 500 | Abdomen | 1 min |
| | side port | | | | |
| I | 1 × 1.5 with side | 20 | 500 | Thigh | 1 min |
| | port | | | | |
| J | 1 × 1.5 without | 20 | 500 | Thigh | 1 min |
| | side port | | | | |
| K | 1 × 1.5 with side | 20 | 500 | Abdomen | 1 min |
| | port | | | | |
| L | 1 × 1.5 without | 20 | 500 | Abdomen | 1 min |
| | side port | | | | |

The Micromedica device was left on the skin for at least one minute following infusion (the “wait” time). If increased leakage is noted due to excess weeping from the device or injection site, the wait time was increased to two minutes for the remainder of the study. Side ported and non-side ported needle infusions of the same psi were run consecutively. Side ported and non side-ported infusions at the same site were administered adjacently, within 3-4 cm of one another. The order of the administration of the injections was randomized prior to the study. Infusions to the anterior thigh region were performed to the left and right of midline. Infusions to the abdomen region were performed to the left and right of umbilicus.

6.3.11.1 Flow Rate

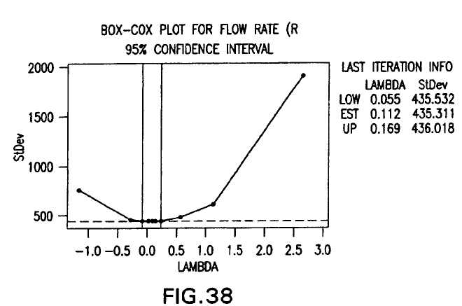

Summary statistics of flow rate measurements per treatment combination are shown below in Table 57. The standard deviations in this table represent the total variability and contain a between donor component. Box Cot plot for flow rate is shown in FIG. 38.

TABLE 57
Summary Statistics for Flow Rate

| | | Experimental Conditions | | | | | | | | | | | |
|---|---|---|---|---|---|---|---|---|---|---|---|---|---|
| | | 10 PSI | | | | 15 PSI | | | | 20 PSI | | | |
| | | Thigh | | Abdomen | | Thigh | | Abdomen | | Thigh | | Abdomen | |
| | | With | No | With | No | With | No | With | No | With | No | With | No |
| | | Side | Side | Side | Side | Side | Side | Side | Side | Side | Side | Side | Side |
| | | Port | Port | Port | Port | Port | Port | Port | Port | Port | Port | Port | Port |
| Flow | Mean | 399.19 | 401.56 | 356.27 | 381.57 | 628.03 | 640.50 | 577.69 | 524.53 | 821.57 | 807.86 | 764.37 | 811.35 |
| Rate | Med | 417.80 | 410.54 | 349.62 | 395.63 | 577.47 | 677.09 | 592.14 | 551.38 | 885.62 | 847.39 | 840.12 | 822.85 |
| (all) | SD | 154.06 | 149.72 | 110.23 | 130.72 | 181.08 | 176.56 | 175.61 | 153.01 | 231.38 | 258.73 | 198.24 | 218.09 |
| (μL/min) | Min | 116.31 | 170.93 | 168.18 | 147.57 | 378.54 | 252.95 | 290.24 | 221.05 | 310.58 | 366.75 | 375.00 | 456.47 |
| | Max | 612.25 | 654.38 | 595.33 | 595.50 | 897.53 | 921.44 | 875.23 | 780.20 | 1176.8 | 1244.6 | 1012.0 | 1093.4 |
| | n | 24 | 24 | 24 | 24 | 24 | 24 | 24 | 24 | 24 | 22 | 24 | 23 |
| Flow | Mean | 399.19 | 401.56 | 356.27 | 381.57 | 634.47 | 640.50 | 577.69 | 534.09 | 821.57 | 807.86 | 764.37 | 811.35 |
| Rate | Med | 417.80 | 410.54 | 349.62 | 395.63 | 613.00 | 677.09 | 592.14 | 552.54 | 885.62 | 847.39 | 840.12 | 822.85 |
| (R² > | SD | 154.06 | 149.72 | 110.23 | 130.72 | 182.32 | 176.56 | 175.61 | 148.95 | 231.38 | 258.73 | 198.24 | 218.09 |
| 0.98) | Min | 116.31 | 170.93 | 168.18 | 147.57 | 378.54 | 252.95 | 290.24 | 221.05 | 310.58 | 366.75 | 375.00 | 456.47 |
| (μL/min) | Max | 612.25 | 654.38 | 595.33 | 595.50 | 897.53 | 921.44 | 875.23 | 780.20 | 1176.8 | 1244.6 | 1012.0 | 1093.4 |
| | n | 24 | 24 | 24 | 24 | 23 | 24 | 24 | 23 | 24 | 22 | 24 | 23 |

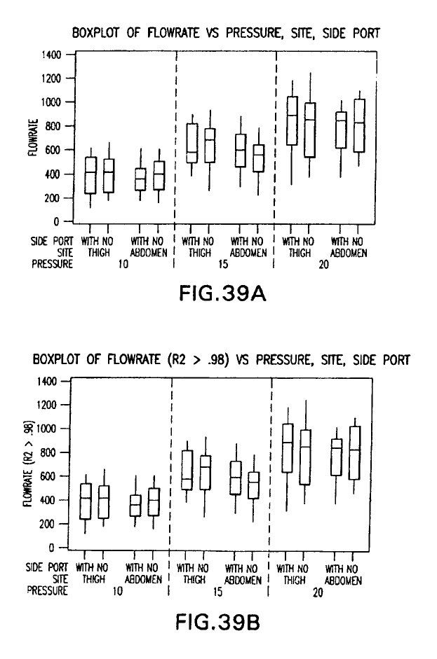

The distribution of flow rate measurements per treatment is shown in FIG. 39.

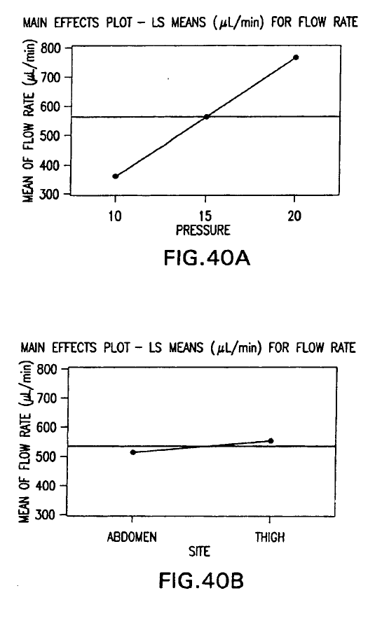

Flow rate (all values of R² and the subset of flow rates with R²>0.98) was analyzed using ANOVA. The ANOVA model included subject-to-subject differences, order of injection, side, side port, site and pressure main effects and 2-way interactions. Because of the non-constant variability/non-normality seen in the residuals, a log transformation was applied to the data and analyzed using ANOVA. The ANOVA results showed that both the site and pressure were significant, but none of the interactions were determined to be significant. The analyses of all flow rate data and of flow rate data with R²>0.98 were similar. With regard to site, the average flow rate for the thigh was significantly higher by 7.2% (95% CI of (1.2%, 12.9%)) than the average flow rate for the Abdomen. As for pressure, the average flow rate increased significantly and steadily by 114.1% (with 95% CI of (95.3%, 134.6%)) between the 10 PSI and 20 PSI pressures. These results are plotted and shown in FIG. 40.

6.3.11.2 Leakage

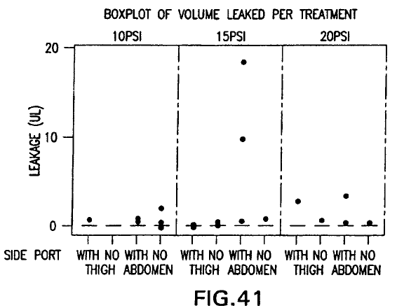

The actual recorded leakage volumes are shown in FIG. 41. There was no occurrence of substantial leakage.

With no occurrence of failure to inject more than 90% of the intended injection volume there was no significant factor effect on the probability of leakage (with a sample size of 24 for each treatment condition, a difference of at least 8% in probability of failure was needed for 90% power of detection between the two device types or sites, and a difference of at least 11.5% in probability of failure was needed for 90% power of detection between the different pressures). Individual 95% upper bounds on the probability of failing to inject at least 90% of 500 μl were calculated for the various treatments. For treatments A-K, with no occurrences of major leakage out of 24 infusions, the 95% upper bound on the probability of failing to inject at least 90% of 500 μl in the thigh was 11.7%. This means that there is a 95% confidence that the chance of failing to inject at least 90% of the intended volume for treatments A-K is no more than 11.7%. For treatment L, with no occurrences of major leakage out of 23 infusions, the 95% upper bound on the probability of failing to inject at least 90% of 500 μl was 12.2%.

6.3.11.3 Pain

Summary statistics for overall perceived pain per treatment combination are shown below in Table 58.

TABLE 58
Summary Statistics for Overall Pain

| | | Experimental Conditions | | | | | | | | | | | |
|---|---|---|---|---|---|---|---|---|---|---|---|---|---|
| | | 10 PSI | | | | 15 PSI | | | | 20 PSI | | | |
| | | Thigh | | Abdomen | | Thigh | | Abdomen | | Thigh | | Abdomen | |
| | | With | No | With | No | With | No | With | No | With | No | With | No |
| | | Side | Side | Side | Side | Side | Side | Side | Side | Side | Side | Side | Side |
| | | Port | Port | Port | Port | Port | Port | Port | Port | Port | Port | Port | Port |
| Overall | Mean | 2.2 | 2.1 | 5.5 | 4.6 | 3.0 | 2.8 | 5.9 | 5.8 | 3.7 | 3.9 | 5.8 | 6.6 |
| Pain | Median | 1 | 1 | 5 | 4 | 1.5 | 2 | 6 | 5 | 2 | 3.5 | 4 | 7 |
| | SD | 2.4 | 2.3 | 3.9 | 4.2 | 3.4 | 2.6 | 4.4 | 3.9 | 3.9 | 3.4 | 4.9 | 4.4 |
| | Min | 0 | 0 | 0 | 0 | 0 | 0 | 0 | 0 | 0 | 0 | 0 | 1 |
| | Max | 8 | 9 | 18 | 20 | 15 | 8 | 18 | 14 | 16 | 11 | 19 | 14 |
| | n | 24 | 24 | 24 | 24 | 24 | 24 | 24 | 24 | 24 | 24 | 24 | 23 |

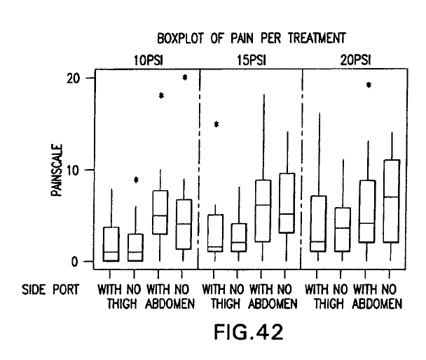

The distribution of pain scores is shown in FIG. 42.

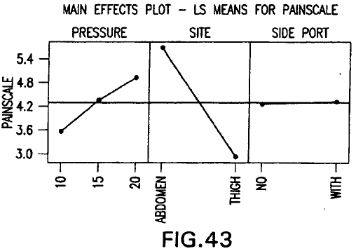

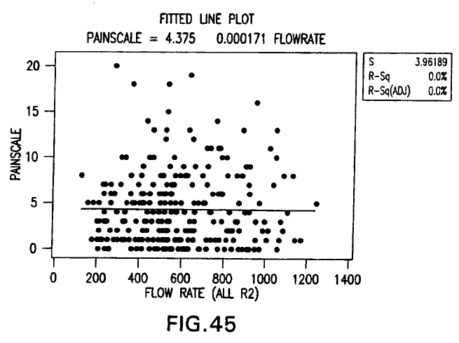

Pain scores were analyzed using ANOVA. The ANOVA model included subject-to-subject differences, order of injection, side, side port, site and pressure main effects and 2-way interactions. The ANOVA results showed that both the site and pressure were significant, but none of the interactions were significant. With regard to the site, the average pain for the abdomen was significantly higher by 2.8 pain scale units (95% CI of (2.1, 3.4)) than the average pain for the thigh. As for pressure, the average pain increased significantly and steadily from an average pain score of 3.6 with 10 PSI to an average pain score of 4.9 with 20 PSI. The size and magnitude of the main effects are shown in FIG. 43, and individual confidence intervals for average pain per device are shown in FIG. 44. In addition, as shown in FIG. 45, no significant relationship between pain and flow rate was observed.

6.3.11.4 Wheal

Table 59 summarizes the number and percent wheals for each experimental condition. A logistic regression was used to investigate the effect of side port, site and pressure on wheal formation, and results showed that site had a significant effect, with abdomen having a higher percentage of wheal formation than thigh.

TABLE 59
Summary of Wheal Formation per Treatment

| | | | | 95% individual |
|---|---|---|---|---|
| | | | | lower bound on |
| Pressure | | | | probability of |
| (PSI) | Site | Side Port | Wheal | wheal |
| 10 | Thigh | Side Port | 17/24 = 70.8% | 52.1% |
| | | No Side Port | 15/24 = 62.5% | 43.7% |
| | Abdomen | Side Port | 23/24 = 95.8% | 81.7% |
| | | No Side Port | 19/24 = 79.2% | 61.1% |
| 15 | Thigh | Side Port | 15/24 = 62.5% | 43.7% |
| | | No Side Port | 15/24 = 62.5% | 43.7% |
| | Abdomen | Side Port | 22/24 = 91.7% | 76.0% |
| | | No Side Port | 24/24 = 100.0% | 88.3% |
| 20 | Thigh | Side Port | 17/24 = 70.8% | 52.1% |
| | | No Side Port | 16/24 = 66.7% | 47.9% |
| | Abdomen | Side Port | 24/24 = 100.0% | 88.3% |
| | | No Side Port | 20/23 = 87.0% | 69.6% |

Summary of wheal formation per site is shown below in Table 60.

TABLE 60
Summary of Wheal Formation

| | | 95% individual lower bound on |
|---|---|---|
| Site | Wheal | probability of wheal |
| Thigh | 95/144 = 66.0% | 58.9% |
| Abdomen | 132/143 = 92.3% | 87.6% |

6.3.11.5 Fluid Observed Upon Removal of Device

Table 61 summarizes the number of times leakage was observed for each device and where the leakage was observed for all injections. A logistic regression was used to investigate the effect of side port, site and pressure on leakage, and results showed that site had a significant effect, with abdomen having a higher percentage of leakage than thigh.

TABLE 61
Summary of Fluid Seen

| Pres- | | | | | | |
|---|---|---|---|---|---|---|
| sure | | | Fluid Seen | | | |
| (PSI) | Site | Side Port | yes | Device | Skin | Both |
| 10 | Thigh | Side Port | 5/24 = 20.8% | 3 | 0 | 2 |
| | | No Side | 3/24 = 12.5% | 1 | 2 | 0 |
| | | Port | | | | |
| | Abdomen | Side Port | 7/24 = 29.2% | 5 | 0 | 2 |
| | | No Side | 11/24 = 45.8% | 6 | 3 | 2 |
| | | Port | | | | |
| 15 | Thigh | Side Port | 3/24 = 12.5% | 2 | 1 | 0 |
| | | No Side | 6/23 = 26.1% | 2 | 2 | 2 |
| | | Port | | | | |
| | Abdomen | Side Port | 7/24 = 29.2% | 5 | 0 | 2 |
| | | No Side | 7/24 = 29.2% | 3 | 1 | 3 |
| | | Port | | | | |
| 20 | Thigh | Side Port | 6/24 = 25.0% | 4 | 0 | 2 |
| | | No Side | 8/24 = 33.3% | 3 | 4 | 1 |
| | | Port | | | | |
| | Abdomen | Side Port | 9/24 = 37.5% | 4 | 2 | 3 |
| | | No Side | 10/22 = 45.5% | 8 | 1 | 1 |

Summary of leakage per site is shown below in Table 62.

TABLE 62
Summary of Leakage per Site

| | | 95% individual upper bound on |
|---|---|---|
| Site | Fluid Seen | probability of fluid seen |
| Thigh | 31/143 = 21.7% | 28.1% |
| Abdomen | 51/142 = 35.9% | 43.1% |

The cause of leakage for eighty (80) of the above “fluid seen” responses was “weeping/pesky drop”. There was one case marked as “mechanical failure/adhesive failure” (for 1×1.5 with side port, 15 PSI, Abdomen) and three cases marked as “mechanical failure/fluid path failure” (one for 1×1.5 with side port, 15 PSI, Thigh, one for 1×1.5 no side port, 10 PSI, Abdomen and one for 1×1.5 no side port, 15 PSI, Abdomen).

6.3.11.6 Safety

Erythema and edema Draize scores are summarized below in Table 65. A binary or ordinal logistic regression was used to investigate the effect of side port, site and pressure on erythema and edema. Results showed that site had a significant effect on erythema and edema scores immediately following the treatment, with the abdomen resulting in more instances of very slight erythema and edema. There was no effect after 24 hours. These results are summarized in the following tables.

TABLE 63
Erythema and Edema Summary
Immediately Following Treatment

| | | | Erythema | | Edema | | |
|---|---|---|---|---|---|---|---|
| | | | | 1 | | 1 | |
| Pressure | | | 0 | Very | 0 | Very | 2 |
| (PSI) | Site | Side Port | None | Slight | None | Slight | Slight |
| 10 | Thigh | Side Port | 23 | 1 | 21 | 3 | 0 |
| | | No Side Port | 23 | 0 | 23 | 1 | 0 |
| | Abdomen | Side Port | 20 | 4 | 8 | 16 | 0 |
| | | No Side Port | 22 | 2 | 10 | 14 | 0 |
| 15 | Thigh | Side Port | 24 | 0 | 21 | 3 | 0 |
| | | No Side Port | 24 | 0 | 22 | 2 | 0 |
| | Abdomen | Side Port | 20 | 4 | 11 | 13 | 0 |
| | | No Side Port | 18 | 5 | 11 | 13 | 0 |
| 20 | Thigh | Side Port | 23 | 1 | 22 | 2 | 0 |
| | | No Side Port | 24 | 0 | 23 | 1 | 0 |
| | Abdomen | Side Port | 22 | 2 | 10 | 13 | 1 |
| | | No Side Port | 22 | 1 | 12 | 11 | 0 |

TABLE 64
Erythema &Edema Summary after 24 hours

| | | | Erythema | | Edema | | |
|---|---|---|---|---|---|---|---|
| | | | | 1 | | 1 | |
| Pressure | | | 0 | Very | 0 | Very | 2 |
| (PSI) | Site | Side Port | None | Slight | None | Slight | Slight |
| 10 | Thigh | Side Port | 24 | 0 | 24 | 0 | 0 |
| | | No Side Port | 24 | 0 | 24 | 0 | 0 |
| | Abdomen | Side Port | 24 | 0 | 24 | 0 | 0 |
| | | No Side Port | 24 | 0 | 24 | 0 | 0 |
| 15 | Thigh | Side Port | 24 | 0 | 24 | 0 | 0 |
| | | No Side Port | 24 | 0 | 24 | 0 | 0 |
| | Abdomen | Side Port | 24 | 0 | 24 | 0 | 0 |
| | | No Side Port | 24 | 0 | 24 | 0 | 0 |
| 20 | Thigh | Side Port | 23 | 1 | 24 | 0 | 0 |
| | | No Side Port | 24 | 0 | 24 | 0 | 0 |
| | Abdomen | Side Port | 24 | 0 | 24 | 0 | 0 |
| | | No Side Port | 23 | 0 | 23 | 0 | 0 |

TABLE 65
Erythema &Edema Summary per Site
Immediately Following Treatment

| | | | Edema | | |
|---|---|---|---|---|---|
| | Erythema | | | 1 | |
| | 0 | 1 | 0 | Very | 2 |
| Site | None | Very Slight | None | Slight | Slight |
| Thigh | 141/143 = | 2/143 = | 132/144 = | 12/144 = | 0 |
| | 98.6% | 1.4% | 91.7% | 8.3% | |
| Abdomen | 124/142 = | 18/142 = | 62/143 = | 80/143 = | 1/143 = |
| | 87.3% | 12.7% | 43.4% | 55.9% | 0.7 |

6.3.11.7 Summary

This 24-subjects study was performed to investigate the 34G×1.5 mm needle with side port placed at 1 mm depth. In particular, the flow rate and success of injection with the 34G-1×1.5 mm needle with side port, using a range of constant pressure forces (10, 15, 20 psi) as compared to the 34G-1×1.5 mm without side port was of primary interest. Two body sites, thigh and abdomen, and a single delivery volume (500 ul) were used. The following results were observed:
Flow rate: Site and pressure were significant. Average flow rate for the abdomen was significantly lower than the average flow rate for the thigh. Average flow rate increased significantly and steadily with increasing pressure.
Leakage: No occurrence of substantial leakage was observed.
Pain: Site and pressure were significant. Average pain for the abdomen was significantly higher than the average pain for the thigh. Average pain increased significantly and steadily with increasing pressure.
Wheal: Site had a significant effect, with abdomen having a higher percentage of wheal formation than thigh.
Fluid Seen: Site had a significant effect, with abdomen having a higher percentage of leakage than thigh.
Erythema and Edema: Site had a significant effect on erythema and edema scores immediately following the treatment, with the abdomen resulting in more instances of very slight erythema and edema than the thigh.

All of the patents, patent applications and references referred to in this application are incorporated in their entirety by reference. Moreover, citation or identification of any reference in this application is not an admission that such reference is available as prior art to this invention. The full scope of this invention is better understood with reference to the appended claims.

1-15. (canceled)

16. A method for delivery of a substance to a human subject's skin comprising depositing the substance into the intradermal compartment of the skin over a period of no more than 10 minutes, at a depth of 1-1.5 mm, at a controlled rate from 5 μL/hr to 5000 μL/min, and at a pressure between 10 psi to 20 psi.

17. The method of claim 16, wherein the pressure is 10 psi.

18. The method of claim 16, wherein the pressure is 15 psi.

19. The method of claim 16, wherein the pressure is 20 psi.

20. The method of claim 16, wherein the delivery rate is controlled using a syringe pump, a microinfusion pump, a Belleville spring, an elastomeric membrane, a gas pressure device, a piezo electric device, an electromotive, or an electromagnetic device.

21. The method of claim 16, wherein the method comprises delivering the substance using a device comprising conventional injection needles, catheters, or microneedles.

22. The method of claim 16, wherein the substance is delivered by a device comprising a needle, said needle having a needle outlet with an exposed height of 0 to 1 mm.

23. The method of claim 16, wherein the method comprises delivering the substance using a device comprising singular or multiple needle arrays.

24. The method of claim 16, wherein the substance is delivered to the subject's thigh.

25. The method of claim 16, wherein the substance is insulin.
